(19) United States
(12) Patent Application Publication
(10) Pub. No.: US 2010/0168441 A1
(43) Pub. Date: Jul. 1, 2010

(54) PROCESS FOR PRODUCTION OF SUBSTITUTED BENZENE

(76) Inventor: Sentaro Okamoto, Kanagawa (JP)

Correspondence Address: BIRCH STEWART KOLASCH & BIRCH, PO BOX 747, FALLS CHURCH, VA, 22040-0747, US

(21) Appl. No.: 12/160,581

(22) PCT Filed: Jan. 9, 2007
(86) PCT No.: PCT/JP2007/050053

§ 371 Date: Jul. 10, 2008

(30) Foreign Application Priority Data
Jan. 11, 2006 (JP) 2006-003216

(51) Int. Cl. C07D 311/76; C07D 493/04; C07C 67/30; C07D 307/87; C07D 209/44; C07F 7/18; C07C 2/48

(52) U.S. Cl. 548/482; 549/458; 560/80; 560/56; 549/469; 549/398; 560/96; 556/443; 585/408

(57) ABSTRACT

Disclosed is a process for production of a substituted benzene, which comprises intramolecularly and/or intermolecularly trimerizing a triple bond in an alkyne in the presence of a transition metal catalyst to yield a substituted benzene compound. In the process, the transition metal catalyst is prepared from an iminomethylpyridine represented by the formula (1) or (2), a transition metal salt or a hydrate thereof, and a reducing agent in a reaction system and is used to perform the trimerization. The process can be used in any one of the intramolecular cyclization of a triyne compound, the cyclization of a diyne compound or an alkyne compound and the intermolecular cyclization of three molecules of an alkyne compound, is excellent in economic effectiveness and operability, and is practically advantageous.

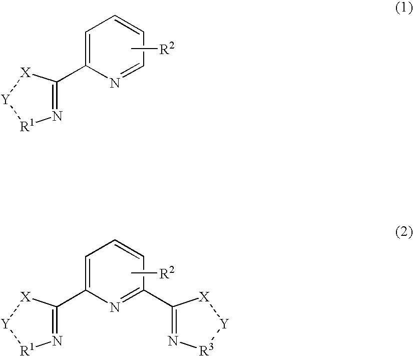

wherein R¹ and R³ independently represent a linear or cyclic C₁-C₂₀ aliphatic hydrocarbon group or the like; R² represents a hydrogen atom or the like; X represents a hydrogen atom, O or the like; and Y represents O, S or the like.

### TECHNICAL FIELD

This invention relates to a production process of substituted benzenes.

### BACKGROUND ART

The trimerization of alkynes continues to be the subject of active research, because it is a reaction of high atom utilization and substituted benzenes or fused benzenes available from the trimerization are important as intermediates for various compounds led by drugs and the like.

Since the finding by Reppe et al. of a process for the direct production of a benzene compound from three acetylene compounds in the presence of a transition metal catalyst, a variety of transition metal complexes have been developed as catalysts for the reaction.

Many of these metal complexes are, however, inferior in economy as they require a costly metal and/or expensive ligand. Moreover, these metal complexes have to be synthesized beforehand upon conducting trimerization, thereby resulting in complex process steps. In addition, special techniques are often needed for procedures such as synthesis and isolation. Therefore, the use of such transition metal complexes can be hardly considered to be a practical method

A method has, hence, been developed in recent years, in which an economical, stable transition metal salt is reduced into an active species of low valency in a reaction system.

For example, as catalysts for a reaction that the three triple bonds in a triyne compound are subjected to intramolecular cyclization to provide a substituted benzene (hereafter called “the type-1 reaction”), CoX₂/Mn catalysts (see Non-patent Documents 1 and 2), CoI₂/PR₃/Mn catalysts (see Non-patent Document 3), FeCl₃ or CoCl₂/imidazolium carbene and Zn catalysts (see Non-patent Document 4) have been reported.

Further, NiX₂/phosphine catalysts (see Non-patent Document 5) have been reported as catalysts usable in both of a reaction that the triple bonds in a diyne compound and an acetylene are subjected to intramolecular and intermolecular cyclization to provide a substituted benzene (hereinafter called “the type-2 reaction”) and a reaction that the triple bonds in three acetylenes are subjected to intermolecular cyclization to provide a substituted benzene (hereinafter called “the type-3 reaction”).

In addition, CoBr₂/2PR₃ and disulfide or diiminenn/Zn/ZnI₂ catalysts (see Non-patent Document 6) have also been reported as catalysts for the type-3 reaction.

However, the catalyst systems of Non-patent Documents 1 to 6 are not applicable to all of the above-mentioned type-1 to type-3 reactions, and their applicable ranges are limited. They are, accordingly, accompanied by a problem that an appropriate catalyst system has to be chosen depending on the substrate. Moreover, many of them need to conduct reactions in water-free systems, and therefore, are hardly usable as industrial production processes.

In addition to the above-described problem, the catalyst systems of Non-patent Document 6 involve another problem that a Co-diimine complex has to be synthesized beforehand.

Non-patent Document 1:
Transition Met. Chem., 1989, 14, 238

Non-patent Document 2:
Transition Met. Chem., 1984, 9, 360

Non-patent Document 3:
Adv. Synth. Catal., 2001, 343, 64

Non-patent Document 4:
Org. Lett., 2005, 7, 3065,

Non-patent Document 5:
J. Org. Chem., 2004, 69, 9224

Non-patent Document 6:
J. Orgmet. Chem., 2005, 690, 5170

### DISCLOSURE OF THE INVENTION

### Problems to be Solved by the Invention

With the foregoing circumstances in view, the present invention has as an object thereof the provision of a practical production process of substituted benzenes, which can be used for all of the above-described type-1 to type-3 reactions and is excellent in economy and operability.

### Means for Solving the Problem

The present inventor conducted an extensive investigation to achieve the above-described object. As a result, it was found that by preparing a catalyst from an iminomethylpyridine, a transition metal salt or a hydrate thereof and a reducing agent in a trimerization reaction system for an alkyne and reacting the alkyne there, each of the above-mentioned type-1 to type-3 reactions proceeds depending upon the employed alkyne and that substituted benzenes can be efficiently obtained from various starting materials in this manner, leading to the completion of the present invention.

Described specifically, the present invention provides:
1. A process for producing a substituted benzene by subjecting a triple bond in an alkyne to intramolecular and/or intermolecular trimerization in the presence of a transition metal catalyst to obtain a substituted benzene compound, wherein the transition metal catalyst is prepared in a reaction system from an iminomethylpyridine represented by the following formula (1) or formula (2), a transition metal salt or a hydrate thereof and a reducing agent to conduct the trimerization.

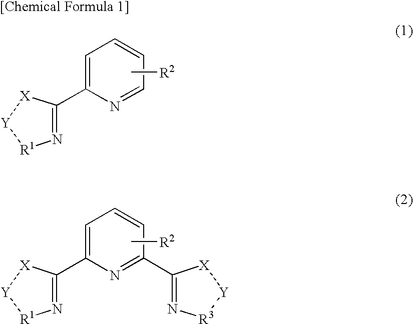

wherein R¹ and R³ each independently represent a linear or cyclic C₁-C₂₀ aliphatic hydrocarbon group or a C₆-C₂₀ aromatic hydrocarbon group, R² represents a hydrogen atom, a linear or cyclic C₁-C₂₀ aliphatic hydrocarbon group or a C₆-C₂₀ aromatic hydrocarbon group, X represents a hydrogen atom, O, S, NR⁴, CH₂, CHR⁴ or CR⁴₂ in which each R⁴ independently represents a linear or cyclic C₁-C₂₀ aliphatic hydrocarbon group or a C₆-C₂₀ aromatic hydrocarbon group, and Y represents O, S, NR⁴, CH₂, CHR⁴ or CR⁴₂ in which each R⁴ independently represents a linear or cyclic C₁-C₂₀ aliphatic hydrocarbon group or a C₆-C₂₀ aromatic hydrocarbon group, with a proviso that, when X is a hydrogen atom, Y is absent and that X and Y do not represent O and/or NR⁴ at a same time.
2. The process as described above in 1, wherein the hydrate of the transition metal salt is represented by the following formula (3):

MZₘ—(H₂O)ₙ (3)

wherein M represents Ti, Zr, V, Nb, Ta, Cr, Mo, W, Mn, Fe, Ru, Co, Rh, Ir, Ni, Pd or Pt, Z represents Cl, Br, I, NO₂, CN, OAc, OBz, OTf, NTf₂, ClO₄, BF₄, PF₆ or acac, in which Ac means an acetyl group, Bz means a benzoyl group, Tf means a trifluoromethanesulfonyl group, and acac means an acetylacetonato group, and m is a number corresponding to a valency of M forming the salt, and n is a number corresponding to a hydrate existing depending on a combination of M and Z.
3. The process as described above in 2, wherein M is Fe, Co, Ni, Pd, Ru or Rh.
4. The process as described above in 2 or 3, wherein Z is Cl, Br or I.
5. The process as described above in 1, wherein the transition metal salt or the hydrate thereof is FeCl₂, FeCl₃, CoCl₂, CoCl₃, NiCl₂, FeCl₃.6H₂O, CoCl₂.6H₂O, or NiCl₂.6H₂O.
6. The process as described above in any one of 1 to 5, wherein the reducing agent is Zn.
7. The process as described above in any one of 1 to 6, wherein the alkyne is a compound represented by the following formula (4), and triple bonds in the compound are subjected to intramolecular trimerization.

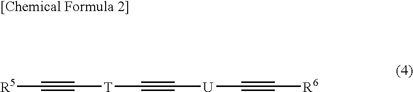

wherein R⁵ and R⁶ each independently represent a hydrogen atom, alkoxy group, hydroxyalkyl group, alkylcarbonyloxy group, amino group, alkoxycarbonyl group, amide group, phosphate ester group, phosphine oxide group, borate ester group, trialkylsilyl group, trialkylstannyl group, linear or cyclic C₁-C₂₀ aliphatic hydrocarbon group, or C₆-C₂₀ aromatic hydrocarbon group in which the aliphatic or aromatic hydrocarbon group may contain at least one of hydroxyl groups, amino groups, alkylcarbonyloxy groups, ether groups, amide groups, cyano groups, nitro groups, phosphate ester groups, phosphine oxide groups, borate ester groups, trialkylsilyl groups, trialkylstannyl groups, dialkylsulfide groups, thiol groups, sulfoxide groups, sulfone groups and sulfonate ester groups, and T and U each independently represent —(CR⁷)ₖ₁—W—, —W—(CR⁷₂)ₖ₁— or —(CR⁷₂)ₖ₂—W—(CR⁷₂)ₖ₃—, in which W represents O, S, NR⁷, SiR⁷₂, BR⁷ or CR⁷₂, each R⁷ independently represents a hydrogen atom, linear or cyclic C₁-C₂₀ aliphatic hydrocarbon group, C₆-C₂₀ aromatic hydrocarbon group or alkoxycarbonyl group, k₁ stands for 2 or 3, and k₂ and k₃ are 1 or 2 and satisfy k₂+k₃=2 or 3.
8. The process as described above in any one of 1 to 6, wherein the alkyne is a combination of a compound represented by the following formula (5) and a compound represented by the following formula (6), and triple bonds in the compounds are subjected to intramolecular and intermolecular trimerization.

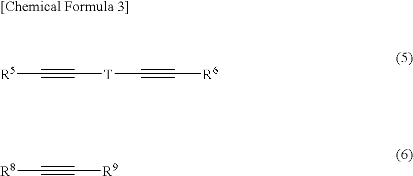

wherein R⁵, R⁶, R⁸ and R⁹ each independently represent a hydrogen atom, alkoxy group, alkylcarbonyloxy group, hydroxyalkyl group, amino group, alkoxycarbonyl group, amide group, phosphate ester group, phosphine oxide group, borate ester group, trialkylsilyl group, trialkylstannyl group, linear or cyclic C₁-C₂₀ aliphatic hydrocarbon group, or C₆-C₂₀ aromatic hydrocarbon group in which the aliphatic or aromatic hydrocarbon group may contain at least one of hydroxyl groups, amino groups, ester groups, ether groups, amide groups, cyano groups, nitro groups, phosphate ester groups, phosphine oxide groups, borate ester groups, trialkylsilyl groups, trialkylstannyl groups, dialkylsulfide groups, thiol groups, sulfoxide groups, sulfone groups and sulfonate ester groups, and T represents —(CR⁷₂)ₖ₁—W—, —W—(CR⁷₂)ₖ₁— or —(CR⁷₂)ₖ₂—W—(CR⁷₂)ₖ₃—, in which W represents O, S, NR⁷, SiR⁷₂, BR⁷ or CR⁷₂, each R⁷ independently represents a hydrogen atom, linear or cyclic C₁-C₂₀ aliphatic hydrocarbon group, C₆-C₂₀ aromatic hydrocarbon group or alkoxycarbonyl group, k₁ stands for 2 or 3, and k₂ and k₃ are 1 or 2 and satisfy k₂+k₃=2 or 3.
9. The process as described above in any one of 1 to 6, wherein the alkyne is a compound represented by the following formula (7), and a triple bond in the compound is subjected to intermolecular trimerization.

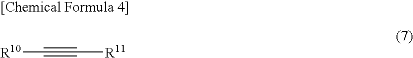

wherein R¹⁰ and R¹¹ each independently represent a hydrogen atom, alkoxy group, hydroxyalkyl group, alkylcarbonyloxy group, amino group, alkoxycarbonyl group, amide group, phosphate ester group, phosphine oxide group, borate ester group, trialkylsilyl group, trialkylstannyl group, linear or cyclic C₁-C₂₀ aliphatic hydrocarbon group, or C₆-C₂₀aromatic hydrocarbon group in which the aliphatic or aromatic hydrocarbon group may contain at least one of hydroxyl groups, amino groups, alkylcarbonyloxy groups, ether groups, amide groups, cyano groups, nitro groups, phosphate ester groups, phosphine oxide groups, borate ester groups, trialkylsilyl groups, trialkylstannyl groups, dialkylsulfide groups, thiol groups, sulfoxide groups, sulfone groups and sulfonate ester groups, with a proviso that R¹⁰ and R¹¹ do not represent hydrogen atoms at a same time in all three molecules.
10. The process as described above in any one of 1 to 9, wherein a silver sulfonate compound selected from the group consisting of AgOSO₂R in which R represents a methyl group, phenyl group, 4-methylphenyl group, trifluoromethyl group or 4-trifluoromethylphenyl group, AgBF₄ and AgPF₆ is added further.
11. The process as described above in 10, wherein the silver sulfonate compound is added in an amount of from 0.2 to 5 equivalents per equivalent of the transition metal salt or the hydrate thereof.

### Effects of the Invention

According to the process of the present invention for the production of a substituted benzene, a transition metal catalyst can be prepared directly in a reaction system from an iminomethylpyridine, a hydrate of a transition metal salt and a reducing agent. It is, therefore, unnecessary to separately synthesize a metal complex, thereby making it possible to simplify the steps and to improve the productivity. Further, the iminomethylpyridine is extremely economical, and as the transition metal, inexpensive one can also be used. Accordingly, the production process of the present invention is also advantageous in cost.

Depending upon the alkyne employed, each of the above-mentioned type-1 to type-3 reactions can proceed, and moreover, no stringent reaction conditions are required because the reaction system is not affected by water.

Moreover, the position selectivity of each substituent in the substituted benzene is often different from that in the conventional processes, thereby making it possible to synthesize compounds the synthesis of which has heretofore been impossible.

The process of the present invention for the production of the substituted benzene, which has such characteristic features as described above, is an extremely useful process as a practical, industrial production process.

### BEST MODES FOR CARRYING OUT THE INVENTION

The present invention will hereinafter be described in further detail. It is to be noted that in this specification, “n” means normal, “i” means iso, “s” means secondary, “t” means tertiary, “c” means cyclo, and “o” means ortho.

The process of the present invention for the production of a substituted benzene is a process for producing the substituted benzene by subjecting a triple bond in an alkyne to intramolecular and/or intermolecular trimerization in the presence of a transition metal catalyst to obtain a substituted benzene compound. The transition metal catalyst is prepared in a reaction system from a ligand composed of an iminomethylpyridine represented by the following formula (1) or formula (2), a hydrate of a transition metal salt and a reducing agent, and the trimerization of the alkyne is conducted.

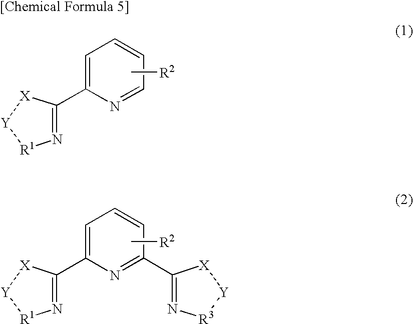

R¹ and R³ each independently represent a linear or cyclic C₁-C₂₀ aliphatic hydrocarbon group, or a C₆-C₂₀ aromatic hydrocarbon group.

Examples of the linear or cyclic C₁-C₂₀ aliphatic hydrocarbon group include alkyl groups such as methyl, ethyl, n-propyl, i-propyl, n-butyl, i-butyl, s-butyl, t-butyl, n-pentyl, c-pentyl, n-hexyl, c-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl and eicosanyl; alkenyl groups such as allyl, 2-butenyl, 3-butenyl, 2-pentenyl, 3-pentenyl, 4-pentenyl, 2-hexenyl, 3-hexenyl, 4-hexenyl, 5-hexenyl, 6-heptenyl, 7-octenyl, 3,7-dimethyl-6-octenyl, 8-nonenyl, 9-decenyl, 10-undecenyl, 11-dodecenyl, 12-tridecenyl, 13-tetradecenyl, 14-pentadecenyl, 15-hexadecenyl, 16-heptadecenyl, 17-octadecenyl, 18-nonadecenyl and 19-eicosenyl; and alkynyl groups such as ethynyl, n-propynyl, i-propynyl, c-propynyl, n-butynyl, i-butyryl, s-butynyl, t-butynyl, c-butynyl, n-pentynyl, 1-methyl-n-butyryl, 2-methyl-n-butynyl, 3-methyl-n-butynyl, 1,1-dimethyl-n-propynyl, c-pentynyl, 2-methyl-c-butynyl, n-hexynyl, 1-methyl-n-pentynyl, 2-methyl-n-pentynyl, 1,1-dimethyl-n-butynyl, 1-ethyl-n-butynyl, 1,1,2-trimethyl-n-propynyl, c-hexynyl, 1-methyl-c-pentynyl, 1-ethyl-c-butynyl, 1,2-dimethyl-c-butyryl, n-heptynyl, n-octynyl, n-nonynyl, n-decynyl, n-undecynyl, n-dodecynyl, n-tridecynyl, n-tetradecynyl, n-pentadecynyl, n-hexadecynyl, n-heptadecynyl, n-octadecynyl, n-nonadecynyl and n-eicosynyl.

Among these, C₁-C₁₀ hydrocarbon groups are preferred, C₁-C₈ hydrocarbon groups are more preferred.

As C₆-C₂₀ aromatic hydrocarbon groups, phenyl, naphthyl and the like can be mentioned.

It is to be noted that in each of these aromatic hydrocarbon groups, at least one hydrogen atom on its ring may be substituted by a substituent. As such substituents, halogen atoms, C₁-C₆ alkyl groups, C₁-C₆ haloalkyl groups, C₁-C₆ alkoxy groups and the like can be mentioned.

As the halogen atoms, fluorine atoms, chlorine atoms, bromine atoms and iodine atoms can be mentioned. The C₁-C₆ alkyl groups can be any ones of linear, branched or cyclic alkyl groups. Illustrative are methyl, ethyl, n-propyl, i-propyl, c-propyl, n-butyl, i-butyl, s-butyl, t-butyl, c-butyl, 1-methyl-c-propyl, 2-methyl-c-propyl, n-pentyl, 1-methyl-n-butyl, 2-methyl-n-butyl, 3-methyl-n-butyl, 1,1-dimethyl-n-propyl, 1,2-dimethyl-n-propyl, 2,2-dimethyl-n-propyl, 1-ethyl-n-propyl, c-pentyl, 1-methyl-c-butyl, 2-methyl-c-butyl, 3-methyl-c-butyl, 1,2-dimethyl-c-propyl, 2,3-dimethyl-c-propyl, 1-ethyl-c-propyl, 2-ethyl-c-propyl, n-hexyl, 1-methyl-n-pentyl, 2-methyl-n-pentyl, 3-methyl-n-pentyl, 4-methyl-n-pentyl, 1,1-dimethyl-n-butyl, 1,2-dimethyl-n-butyl, 1,3-dimethyl-n-butyl, 2,2-dimethyl-n-butyl, 2,3-dimethyl-n-butyl, 3,3-dimethyl-n-butyl, 1-ethyl-n-butyl, 2-ethyl-n-butyl, 1,1,2-trimethyl-n-propyl, 1,2,2-trimethyl-n-propyl, 1-ethyl-l-methyl-n-propyl, 1-ethyl-2-methyl-n-propyl, c-hexyl, 1-methyl-c-pentyl, 2-methyl-c-pentyl, 3-methyl-c-pentyl, 1-ethyl-c-butyl, 2-ethyl-c-butyl, 3-ethyl-c-butyl, 1,2-dimethyl-c-butyl, 1,3-dimethyl-c-butyl, 2,2-dimethyl-c-butyl, 2,3-dimethyl-c-butyl, 2,4-dimethyl-c-butyl, 3,3-dimethyl-c-butyl, 1-n-propyl-c-propyl, 2-n-propyl-c-propyl, 1-i-propyl-c-propyl, 2-i-propyl-c-propyl, 1,2,2-trimethyl-c-propyl, 1,2,3-trimethyl-c-propyl, 2,2,3-trimethyl-c-propyl, 1-ethyl-2-methyl-c-propyl, 2-ethyl-l-methyl-c-propyl, 2-ethyl-2-methyl-c-propyl, 2-ethyl-3-methyl-c-propyl, and the like.

The C₁-C₆ alkoxy groups can be any ones of linear, branched or cyclic alkoxy groups. Illustrative are methoxy, ethoxy, n-propoxy, i-propoxy, c-propoxy, n-butoxy, i-butoxy, s-butoxy, t-butoxy, c-butoxy, 1-methyl-c-propoxy, 2-methyl-c-propoxy, pentoxy, c-pentoxy, hexoxy, c-hexoxy, and the like.

As the C₁-C₆ haloalkoxy groups, can be mentioned those formed by substituting at least one hydrogen atoms of the above-described C₁-C₆ alkyl groups with halogen atoms.

Specific examples of aromatic hydrocarbon groups having substituents include o-methylphenyl, m-methylphenyl, p-methylphenyl o-trifluoromethylphenyl, m-trifluoromethylphenyl, p-trifluoromethylphenyl, p-ethylphenyl, p-i-propylphenyl, p-t-butylphenyl, 2,4,5-trimethylphenyl, 2,5-di-i-propylphenyl, o-chlorophenyl, m-chlorophenyl, p-chlorophenyl, o-bromophenyl, m-bromophenyl, p-bromophenyl, o-fluorophenyl, p-fluorophenyl, o-methoxyphenyl, m-methoxyphenyl, p-methoxyphenyl, o-trifluoromethoxyphenyl, p-trifluoromethoxyphenyl, o-nitrophenyl, m-nitrophenyl, p-nitrophenyl, o-dimethylaminophenyl, m-dimethylaminophenyl, p-dimethylaminophenyl, p-cyanophenyl, 3,5-dimethylphenyl, 3,5-bistrifluoromethylphenyl, 3,5-dimethoxyphenyl, 3,5-bistrifluoromethoxyphenyl, 3,5-diethylphenyl, 3,5-di-i-propylphenyl, 3,5-dichlorophenyl, 3,5-dibromophenyl, 3,5-difluorophenyl, 3,5-dinitrophenyl, 3,5-dicyanophenyl, 2,4,6-trimethylphenyl, 2,4,6-tristrifluoromethylphenyl, 2,4,6-trimethoxyphenyl, 2,4,6-tristrifluorometoxyphenyl, 2,4,6-trichlorophenyl, 2,4,6-tribromophenyl, 2,4,6-trifluorophenyl, α-naphthyl, β-naphthyl, o-biphenylyl, m-biphenylyl, p-biphenylyl, and the like.

R² represents a hydrogen atom, a linear or cyclic C₁-C₂₀ aliphatic hydrocarbon group, or a C₆-C₂₀ aromatic hydrocarbon group. Specific examples of these hydrocarbon groups are as described above.

X represents a hydrogen atom, O, S, NR⁴, CH₂, CHR⁴ or CR⁴₂, and Y represents O, S, NR⁴, CH₂, CHR⁴ or CR⁴₂.

Each R⁴ represents a linear or cyclic C₁-C₂₀ aliphatic hydrocarbon group or a C₆-C₂₀ aromatic hydrocarbon group. Specific examples of these hydrocarbon groups are as described above, however, when R⁴ represents an aliphatic hydrocarbon group, a C₁-C₁₀ aliphatic hydrocarbon group is preferred, a C₁-C₆ aliphatic hydrocarbon group is more preferred.

It is to be noted that, when X is a hydrogen atom, Y is absent because no ring is formed. It is also to be noted that X and Y do not represent O and/or NR⁴ at a same time.

Specific non-limited examples of the iminomethylpyridine represented by the formula (1) or formula (2) include the following iminomethylpyridines:

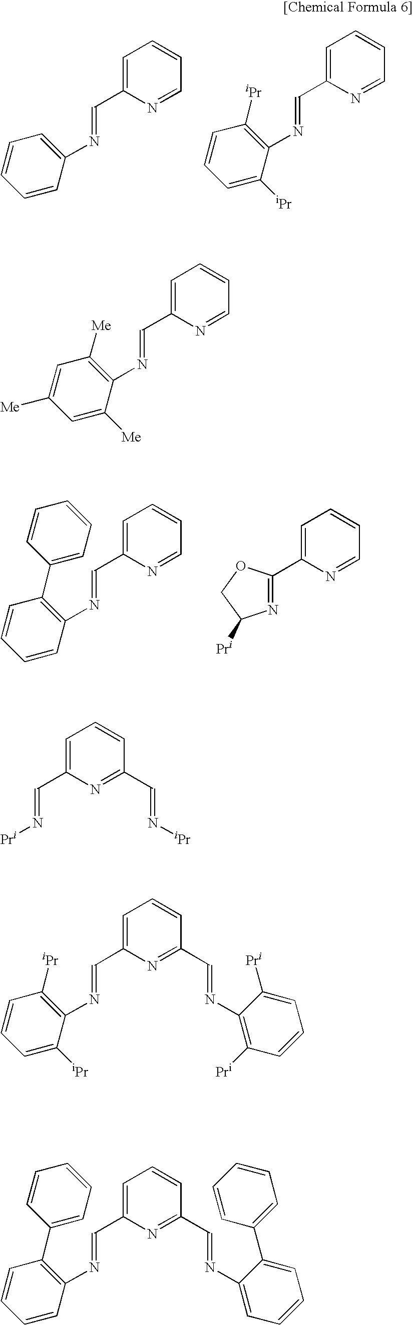

wherein Me means a methyl group and ⁱPr means an isopropyl group, and the same shall apply hereinafter.

No particular limitation is imposed on the transition metal salt or its hydrate to be used for the preparation of the catalyst (metal complex), and various metal salts and their hydrates which have conventionally been employed in this sort of reactions are each usable. For example, those represented by the following formula (3) or (3′) can be mentioned, although the use of a hydrate of the formula (3) is preferred in the production process according to the present invention.

MZₘ—(H₂O)ₙ (3)

MZₘ (3′)

In the formulas (3) and (3′), M represents Ti, Zr, V, Nb, Ta, Cr, Mo, W, Mn, Fe, Ru, Co, Rh, Ir, Ni, Pd or Pt. Taking the catalytic activity and the like into consideration, Fe, Co, Ni, Pd, Ru and Rh are preferred. Taking the production cost into further consideration, Fe, Co and Ni are more preferred.

Z represents Cl, Br, I, NO₂, CN, OAc, OBz, OTf, NTf₂, ClO₄, BF₄, PF₆ or acac, in which Ac means an acetyl group, Bz means a benzoyl group, Tf means a trifluoromethanesulfonyl group, and acac means an acetylacetonato group. Taking the availability as the hydrate of the salt into consideration, Cl, Br and I are preferred.

Further, m is a number corresponding to the valency of M forming the salt, and n in the formula (3) is a number corresponding to a hydrate existing depending on a combination of M and Z, and cannot be specified sweepingly.

As transition metal salts suitably usable in the production process of the present invention, FeCl₂, FeCl₃, CoCl₂, CoCl₃, NiCl₂ and the like can be mentioned.

As hydrates of transition metal salts, on the other hand, FeCl₂.4H₂O, FeI₂.4H₂O, FeCl₃.6H₂O, CoCl₂.6H₂O, CoBr₂.6H₂O, NiCl₂.6H₂O, NiBr₂.6H₂O and the like can be mentioned.

No particular limitation is imposed on the reducing agent insofar as it can reduce the above-mentioned transition metal to form an active species in the system. Illustrative are metals such as Li, Na, K, Mg, Ca, Al, Mn, Zn and Sm; and organometal compounds such as R⁴Li, R⁴K, R⁴MgHal, R⁴₂Mg, R⁴ZnHal, R⁴₂Zn, R⁴₃Al, R⁴₂AlHal, and R⁴AlHal₂ in which each R⁴ has the same meaning as defined above and each Hal represents a halogen atom. Among these, Mg, Mn, Zn and Al are preferred with Zn being more preferred, from the standpoints of stability, easiness of handling in the air, low-cost, and the readiness and safety of separation by filtration after completion of the reaction.

The above-described metals can each be used in a desired form. In general, it is used in a powder form. The organometal compounds, on the other hand, can be used either neat or as solutions.

In the process of the present invention for the production of a substituted benzene in which a metal complex having an iminomethylpyridine ligand is used as a catalyst, a triyne compound of the below-described formula (4), a combination of a diyne compound of the below-described formula (5) and an acetylene compound of the below-described formula (6), or three molecules of an acetylene compound of the below-described formula (7) can be used as an alkyne, that is, a reaction substrate. By selectively using these alkynes, the above-mentioned type-1 to type-3 trimerization reactions all proceed.

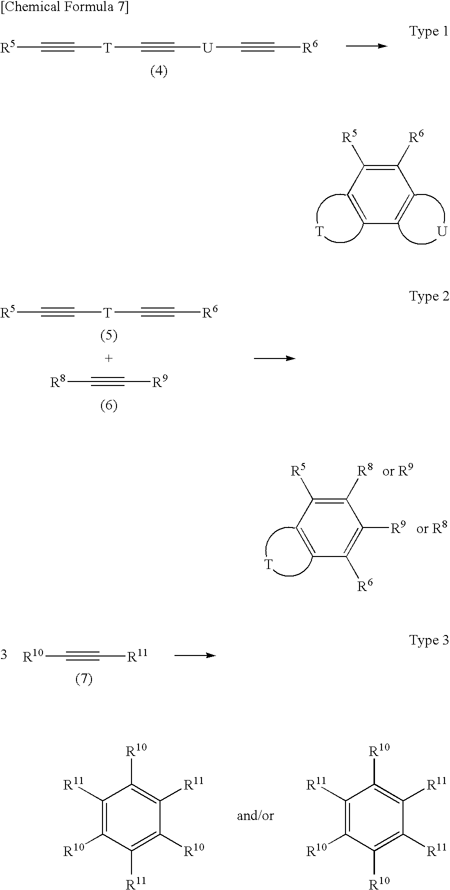

Described specifically, in the case of the compound represented by the formula (4), the three triple bonds which this compound has are intramolecularly trimerized to form a fused-ring, substituted benzene.

When the compound represented by the formula (5) and the compound represented by the formula (6) are used in combination, the respective triple bonds in these compounds are intramolecularly and intermolecularly trimerized to form a fused-ring, substituted benzene.

In the case of the three molecules of the compound represented by the formula (7), the triple bonds in these compounds are intermolecularly trimerized to form a substituted benzene.

In the formulas (4) to (6), R⁵ to R⁹ each independently represent a hydrogen atom, alkoxy group, hydroxyalkyl group, alkylcarbonyloxy group, amino group, alkoxycarbonyl group, amide group, phosphate ester group, phosphine oxide group, borate ester group, trialkylsilyl group, trialkylstannyl group, linear or cyclic C₁-C₂₀ aliphatic hydrocarbon group, or C₆-C₂₀ aromatic hydrocarbon group in which the aliphatic or aromatic hydrocarbon group may contain at least one of hydroxyl groups, amino groups, alkylcarbonyloxy groups, ether groups, amide groups, cyano groups, nitro groups, phosphate ester groups, phosphine oxide groups, borate ester groups, trialkylsilyl groups, trialkylstannyl groups, dialkylsulfide groups, thiol groups, sulfoxide groups, sulfone groups and sulfonate ester groups.

As the hydroxyalkyl group, one containing a hydroxyl group bonded to a desired carbon atom in a C₁-C₂₀ alkyl group can be mentioned. Specific examples include hydroxymethyl, hydroxyethyl, hydroxypropyl, and 1,2-dihydroxyethyl.

Examples of the alkylcarbonyloxy group include methylcarbonyloxy, ethylcarbonyloxy, n-propylcarbonyloxy, i-propylcarbonyloxy, n-butylcarbonyloxy, s-butylcarbonyloxy, t-butylcarbonyloxy, n-pentylcarbonyloxy, n-hexylcarbonyloxy, and the like.

Examples of the alkoxycarbonyl group include methoxycarbonyl, ethoxycarbonyl, n-propoxycarbonyl, i-propoxycarbonyl, n-butoxycarbonyl, s-butoxycarbonyl, t-butoxycarbonyl, n-pentyloxycarbonyl, n-hexyloxycarbonyl, and the like.

Examples of the trialkylsilyl group include trimethylsilyl, triethylsilyl, triisopropylsilyl, diethylisopropylsilyl, dimethylisopropylsilyl, di-t-butylmethylsilyl, isopropyldimethylsilyl, t-butyldimethylsilyl, thexyldimethylsilyl, and the like.

Examples of the trialkylstannyl group include trimethylstannyl, triethylstannyl, tri-n-propylstannyl, triisopropylstannyl, tri-n-butylstannyl, triisobutylstannyl, tri-s-butylstannyl, tri-t-butylstannyl, and the like.

As specific examples of the linear or cyclic C₁-C₂₀ aliphatic hydrocarbon group and C₆-C₂₀ aromatic hydrocarbon group, same groups as those exemplified above can be mentioned. As alkoxy groups, same groups as those exemplified above with respect to the C₁-C₆ alkoxy group can be mentioned.

In the formulas (4) and (5), T and U each independently represent —(CR⁷₂)ₖ₁—W—, —W—(CR⁷₂)ₖ₁— or —(CR⁷)ₖ₂—W—CR₂⁷)ₖ₃—.

Here, W represents O, S, NR⁷, SiR⁷₂, BR⁷ or CR⁷₂, each R⁷ independently represents a hydrogen atom, linear or cyclic C₁-C₂₀ aliphatic hydrocarbon group, C₆-C₂₀ aromatic hydrocarbon group or alkoxycarbonyl group, k₁ stands for 2 or 3, and k₂ and k₃ are 1 or 2 and satisfy k₂+k₃=2 or 3. In other words, T and U can each form a 5-membered ring or 6-membered ring when the triple bonds on its opposite sides react.

It is to be noted that as specific examples of the linear or cyclic C₁-C₂₀ aliphatic hydrocarbon group, C₆-C₂₀ aromatic hydrocarbon group and alkoxycarbonyl group, groups similar to those exemplified above can be mentioned.

In the formula (7), R¹⁰ and R¹¹ each independently represent a hydrogen atom, alkoxy group, hydroxyalkyl group, amino group, alkylcarbonyloxy group, alkoxycarbonyl group, amide group, phosphate ester group, phosphine oxide group, borate ester group, trialkylsilyl group, trialkylstannyl group, linear or cyclic C₁-C₂₀ aliphatic hydrocarbon group, or C₆-C₂₀ aromatic hydrocarbon group in which the aliphatic or aromatic hydrocarbon group may contain at least one of hydroxyl groups, amino groups, alkylcarbonyloxy groups, ether groups, amide groups, cyano groups, nitro groups, phosphate ester groups, phosphine oxide groups, borate ester groups, trialkylsilyl groups, trialkylstannyl groups, dialkylsulfide groups, thiol groups, sulfoxide groups, sulfone groups and sulfonate ester groups. However, R¹⁰ and R¹¹ do not represent hydrogen atoms at the same time in all the three molecules because the reaction product is a substituted benzene in the present invention. In other words, at least one of the six R¹⁰ and R¹¹ in total in the three molecules is a substituent other than a hydrogen atom.

As specific examples of such substituent or substituents, substituents same as those exemplified above can be mentioned.

As specific examples of the compound represented by the formula (4), the following compounds can be mentioned although it is not limited to them.

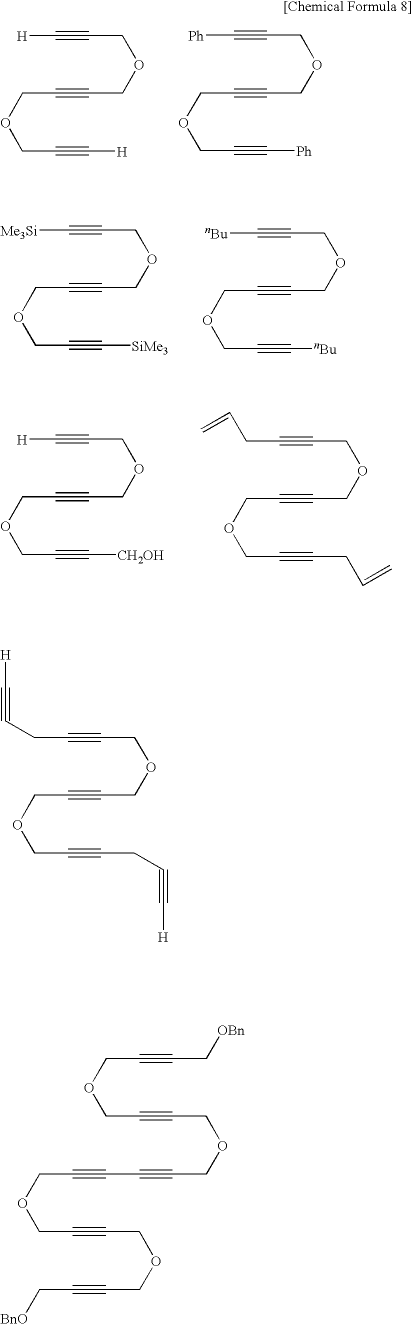

wherein ⁿBu means a normal butyl group, Ph means a phenyl group, and Bn means a benzyl group, and the same shall apply hereinafter.

As specific examples of the compound represented by the formula (5), the following compounds can be mentioned although it is not limited to them.

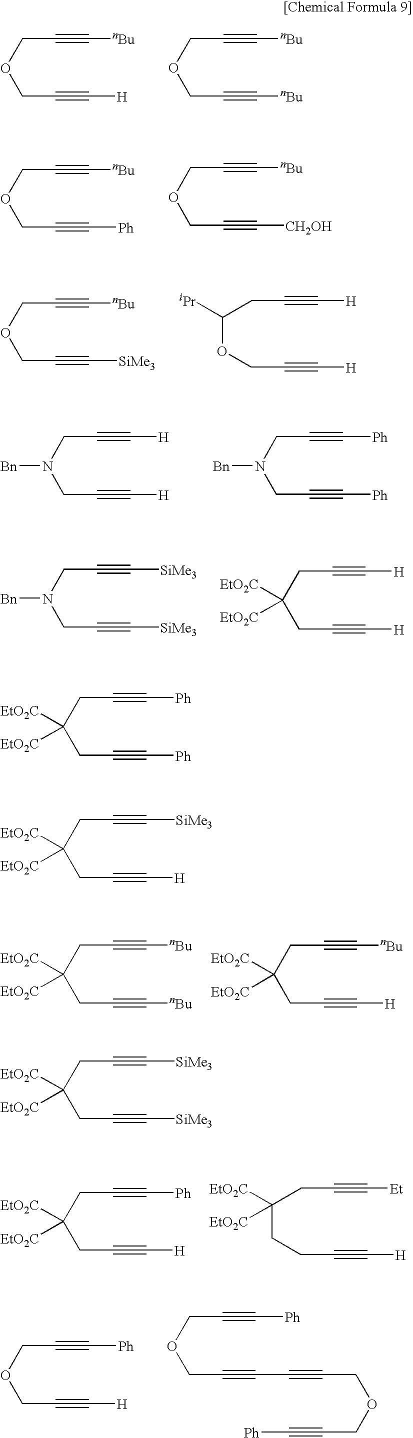

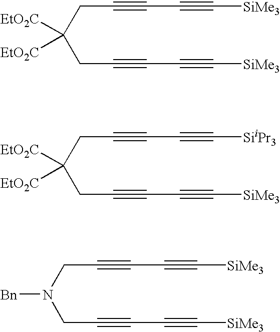

wherein Et means an ethyl group, and the same shall apply hereinafter.

As specific examples of the compounds represented by the formulas (6) and (7), the following compounds can be mentioned although they are not limited to the following compounds.

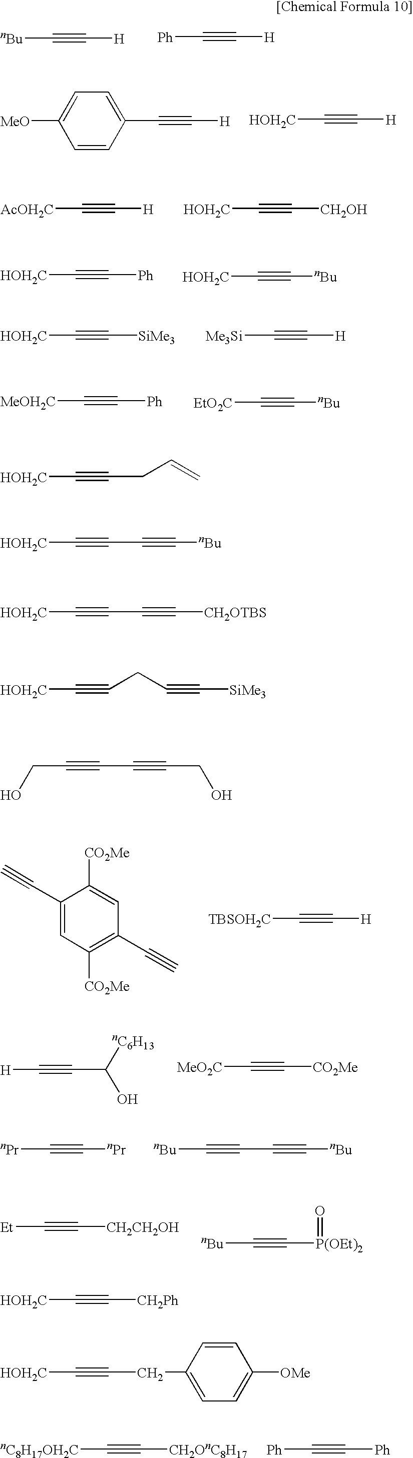

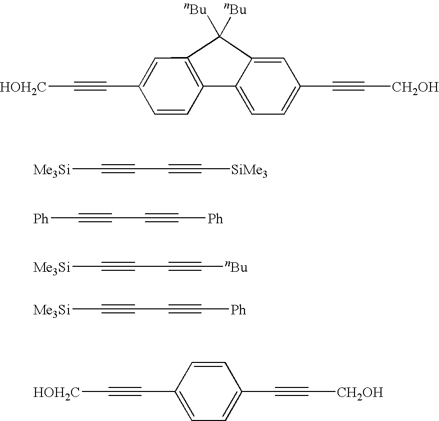

wherein ⁿPr means a normal propyl group, Ac means an acetyl group, and TBS means a t-butyldimethylsilyl group, and the same shall apply hereinafter.

A description will be now made about reaction conditions for the process according to the present invention for the production of a substituted benzene.

No particular limitations are imposed on the amounts of the starting materials to be used for the preparation of the transition metal catalyst insofar as they fall within their corresponding ranges that can form a complex. In general, the iminomethylpyridine may be used in an amount of from 0.5 to 3 equivalents or so, preferably from 0.7 to 2 equivalents, more preferably from 1 to 1.3 equivalents per equivalent of the transition metal salt or its hydrate.

On the other hand, the amount of the reducing agent to be used may be from 0.5 to 20 equivalents or so, preferably from 0.7 to 10 equivalents, more preferably from 1 to 5 equivalents based on the transition metal salt or its hydrate.

No particular limitation is imposed on the amount of the transition metal catalyst to be used in the trimerization of the alkyne insofar as the trimerization proceeds. In general, the transition metal catalyst may be used at from 0.01 to 50 mol % or so, preferably from 1 to 15 mol %, more preferably from 1 to 5 mol % in terms of the metal salt or its hydrate based on the whole alkyne or alkynes to be used.

When the above-mentioned type-2 reaction is conducted, the diyne and acetylene may generally be used at a ratio of from 0.5 to 3 equivalents of the diyne to from 0.5 to 10 equivalents or so of the acetylene. Preferably, the acetylene may be used as much as from 0.5 to 3 equivalents per equivalent of the diyne.

In the production process of the present invention, no reaction solvent may be used. When a solvent is used, any one of various solvents conventionally employed in organic syntheses can be used insofar as it does not deleteriously affect the reaction.

Specific examples include water, alcohols (methanol, ethanol, propanol, butanol, octanol, etc.), cellosolves (methoxyethanol, ethoxyethanol, etc.), aprotonic polar organic solvents (dimethylformamide, dimethylsulfoxide, dimethylacetamide, tetramethylurea, sulfolane, N-methylpyrrolidone, N,N-dimethylimidazolidinone, etc.), ethers (diethyl ether, diisopropyl ether, t-butyl methyl ether, tetrahydrofuran, dioxane, etc.), aliphatic hydrocarbons (pentane, hexane, c-hexane, octane, decane, decalin, petroleum ether, etc.), aromatic hydrocarbons (benzene, chlorobenzene, o-dichlorobenzene, nitrobenzene, toluene, xylene, mesitylene, tetralin, etc.), halogenated hydrocarbons (chloroform, dichloromethane, dichloroethane, carbon tetrachloride, etc.), ketones (acetone, methyl ethyl ketone, methyl butyl ketone, methyl isobutyl ketone, etc.), lower fatty acid esters (methyl acetate, ethyl acetate, butyl acetate, methyl propionate, etc.), alkoxyalkanes (dimethoxyethane, diethoxyethane, etc.), and nitriles (acetonitrile, propionitrile, butyronitrile, etc.). They can be used either singly or in combination.

Among these solvents, at least one solvent selected from the ethers, nitriles and water is preferred with the single solvent of tetrahydrofuran (hereinafter referred to as “THF”) or a mixed solvent of THF and water being particularly preferred, in view of its solubility for the alkyne as a substrate and the catalyst, its safety and cost and the readiness of its separation from the substituted benzene as the reaction product.

In the process of the present invention for the production of a substituted benzene, it is possible to add, as an additive, a silver sulfonate compound selected from the group consisting of AgOSO₂R, in which R represents a methyl group, phenyl group, 4-methylphenyl group, trifluoromethyl group or 4-trifluoromethylphenyl group, AgBF₄ and AgPF₆, preferably silver trifluoromethanesulfonate. By conducting the reaction with such a silver sulfonate compound added therein, the trimerization of the alkyne is promoted, and moreover, the reaction readily proceeds even on a substrate which is hardly reactable without an additive.

In this case, the amount of the silver sulfonate compound to be added may be set preferably at from 0.2 to 5 equivalents, more preferably from 0.5 to 3 equivalents per equivalent of the transition metal salt or its hydrate to be used.

Upon conducting the reaction, the starting materials for the preparation of the transition metal catalyst, the alkyne as a reaction substrate, and the silver sulfonate compound as an additive can be mixed in a desired order. For example, all the reagents may be mixed at once to perform the preparation of the catalyst and the trimerization substantially at the same time. As an alternative, the transition metal salt or its hydrate, the iminomethylpyridine and the reducing agent, which are starting materials for the preparation of the transition metal catalyst, may be firstly mixed in a desired order to prepare the transition metal catalyst, and the silver sulfonate compound and alkyne may then be added into the system to conduct the trimerization.

The trimerization can be conducted under an atmosphere of deoxygenated air, nitrogen gas, argon gas, carbon dioxide gas or helium gas, especially preferably under an atmosphere of argon gas or nitrogen gas.

The reaction temperature may be generally from 0 to 150° C. or so, preferably from 10 to 120° C. or so, more preferably from 20 to 50° C. The reaction time may generally be from 0.1 to 100 hours.

After completion of the reaction, the target product is extracted with a suitable solvent, and the solvent is evaporated under reduced pressure to obtain the substituted benzene compound in a crude form. Purification is then performed by a usual method such as chromatography on a silica gel column to isolate the substituted benzene compound in a pure form.

### Examples

The present invention will hereinafter be specifically described based on Examples, although the present invention shall not be limited to the following Examples. It is to be noted that the physical properties of each compound in the following description were measured using the following systems.
[1] ¹H, ¹³C or ³¹P-NMR spectrum

Measured by “JNM-ECA600”, “JNM-ECA500” or “JNM-EX270” (all manufactured by JEOL Ltd.).
[2] IR spectrum

Measured by “FT-IR(270-30)” (manufactured by Hitachi, Ltd.).

### Example 1

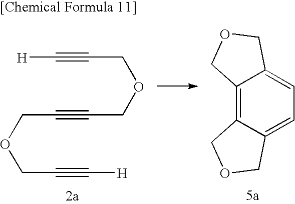

Zinc powder (6.5 mg, 0.10 mmol) and the compound 2a (1.0 mmol) were dissolved in THF (2.5 mL), followed by the addition of a solution of CoCl₂-6H₂O (11.9 mg, 0.05 mmol) and 2-(2,6-diisopropylphenyl)iminomethylpyridine (hereinafter referred to as “dipimp”, 16.0 mg, 0.06 mmol) in THF (1.5 mL). The resulting mixed solution was warmed for 5 minutes at 35 to 40° C., and was then stirred at room temperature. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzene 5a (yield: 62%).

¹H-NMR (500 MHz, CDCl₃): δ 7.15 (s, 2H, Ar), 5.13 (s, 4H, CH₂), 5.04 (s, 4H, CH₂).

¹³C-NMR (125 MHz, CDCl₃): δ 138.6, 132.3, 119.9, 73.4, 72.2.

IR (KBr): 2854, 1464, 1386, 1038, 1018 cm⁻¹.

Mp: 83-85° C.

Anal. Calcd for C₁₀H₁₀O₂: C, 74.06; H, 6.21. Found: C, 73.70; H, 6.09.

It is to be noted that dipimp represented by the following formula was synthesized from 2,6-diisopropylaniline and pyridine 2-carboxyaldehyde in accordance with the procedure described in Organometallics, 1994, 13, 3990, and J. Organomet. Chem. 2005, 690, 5170.

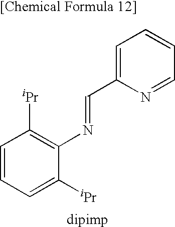

### Example 2

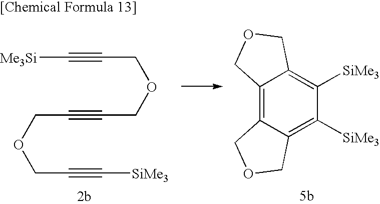

The substituted benzene 5b was obtained in a similar manner as in Example 1 except that zinc powder (6.5 mg, 0.10 mmol) and the compound 2b (1.0 mmol) were dissolved in THF (2.5 mL) (yield: 97%).

¹H-NMR (500 MHz, CDCl₃): δ 5.17 (s, 4H, CH₂), 4.95 (s, 4H, CH₂), 0.40 (s, 18H, SiMe₃).

¹³C-NMR (125 MHz, CDCl₃): δ 145.6, 138.9, 132.4, 71.2, 3.6.

IR (neat): 2950, 2900, 2855, 1728, 1251, 1059 cm⁻¹.

Mp: 134-136° C.

Anal. Calcd for Found: C₁₆H₂₆O₂Si₂: C, 62.69; H, 8.55. Found: C, 62.80; H, 8.48.

### Example 3

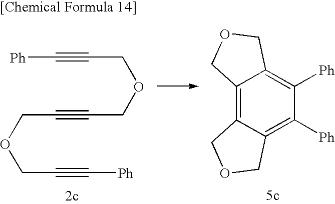

The substituted benzene 5c was obtained from the compound 2c in a similar manner as in Example 2 (yield: 82%).

¹H-NMR (500 MHz, CDCl₃): δ 6.98-7.22 (m, 10H, Ph), 5.14 (s, 4H, CH₂), 4.99 (s, 4H, CH₂).

¹³C-NMR (125 MHz, CDCl₃): δ 138.7, 138.4, 134.1, 131.2, 129.4, 127.9, 126.8, 73.6, 72.7.

IR (KBr): 2922, 2847, 1446, 1429, 1352, 1063, 1043 cm⁻¹.

Mp: 188-193° C.

Anal. Calcd for C₂₂H₁₈O₂: C, 84.05; H, 5.77. Found: C, 83.71; H, 5.76.

### Example 4

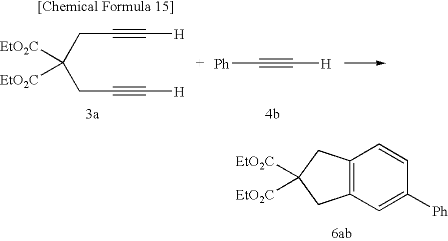

Zinc powder (6.5 mg, 0.10 mmol), the compound 3a (236 mg, 1.0 mmol) and the compound 4b (133 mg, 1.3 mmol) were dissolved in THF (2.5 mL), followed by the addition of a solution of CoCl₂-6H₂O (11.9 mg, 0.05 mmol) and dipimp (16.0 mg, 0.06 mmol) in THF (1.5 mL). The resulting mixed solution was warmed for 5 minutes at 35 to 40° C., and was then stirred at room temperature for 4 hours. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzene 6ab (yield: 91%).

¹H-NMR (600 MHz, CDCl₃): δ 7.55 (d, 2H, J=7.2 Hz, Ar), 7.35-7.43 (m, 4H, Ar), 7.32 (t, 1H, J=7.2 Hz, Ar), 7.25 (d, 1H, J=7.2 Hz, Ar), 4.22 (q, 4H, J=7.2 Hz, OCH₂CH₃), 3.65 (s, 2H, ArCH₂C), 3.63 (s, 2H, ArCH₂C), 1.26 (t, 6H, J=7.2 Hz, OCH₂CH₃).

¹³C-NMR (150 MHz, CDCl₃): δ 171.6, 141.3, 140.7, 139.1, 128.6, 127.1, 127.0, 126.1, 124.4, 123.0, 67.9, 61.7, 60.5, 40.4, 40.2, 14.0.

IR (neat): 3030, 2980, 2938, 1725, 1712, 1599, 1570, 1485, 1242, 1184, 1157 cm⁻¹.

Anal. Calcd for C₂₁O₂₂O₄: C, 74.54; H, 6.55. Found: C, 74.59, H, 6.55.

### Example 5

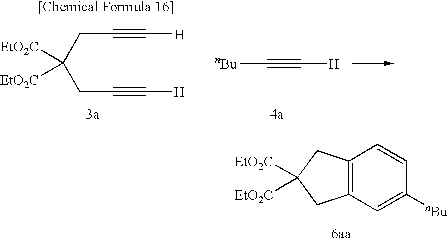

The substituted benzene 6aa was obtained from the compound 3a and compound 4a in a similar manner as in Example 4 except for the use of the compound 4a (3 mmol) (yield: 63%).

¹H-NMR (600 MHz, CDCl₃): δ 7.08 (d, 1H, J=7.8 Hz, Ar), 7.01 (s, 1H, Ar), 6.97 (d, 1H, J=7.8 Hz, Ar), 4.20 (q, 4H, J=7.2 Hz, OCH₂CH₃), 3.56 (s, 2H, ArCH₂C), 3.55 (s, 2H, ArCH₂C), 2.56 (t, 2H, J=7.8 Hz, ArCHhd 2CH₂), 1.56 (quint, 2H, J=7.8 Hz, CH₂CH₂CH₃), 1.34 (sext, 2H, J=7.8 Hz, CH₂CH₂CH₃), 1.25 (t, 6H, J=7.2 Hz, OCH₂CH₃), 0.91 (t, 3H, J=7.8 Hz, CH₂CH₂CH₃).

¹³C-NMR (150 MHz, CDCl₃): δ 171.8, 141.7, 140.0, 137.1, 127.1, 124.1, 123.8, 61.6, 60.6, 40.4, 40.1 35.5, 33.8, 22.4, 14.0, 13.9.

IR (neat): 2980, 2959, 2932, 2859, 1738, 1725, 1248, 1184, 1157 cm⁻¹.

Anal. Calcd for C₁₉H₂₆O₄: C, 71.67; H, 8.23. Found: C, 71.77; H, 8.34.

### Example 6

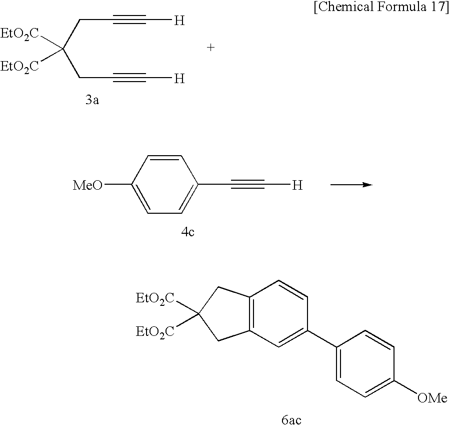

The substituted benzene 6ac was obtained from the compound 3a and compound 4c in a similar manner as in Example 4 except for the use of the compound 4c (1.3 mmol) (yield: 92%).

¹H-NMR (600 MHz, CDCl₃): δ 7.48 (d, 2H, J=7.8 Hz, Ar), 7.36 (s, 1H, Ar), 7.35 (d, 1H, J=7.8 Hz, Ar), 7.23 (d, 1H, J=7.8 Hz, Ar), 6.95 (d, 2H, J=7.8 Hz, Ar), 4.22 (q, 4H, J=6.6 Hz, OCH₂CH₃), 3.84 (s, 3H, OMe), 3.63 (s, 2H, ArCH₂C), 3.61 (s, 2H, ArCH₂C), 1.26 (t, 6H, J=6.6 Hz, OCH₂CH₃).

¹³C-NMR (150 MHz, CDCl₃): δ 171.6, 158.9, 140.6, 139.9, 138.5, 133.8, 128.1, 125.7, 124.4, 122.5, 114.1, 61.7, 60.5, 55.3, 40.5, 40.1, 14.0.

IR (neat): 2982, 2938, 2907, 1728, 1242, 1179, 1153 cm⁻¹.

Anal. Calcd for C₂₂H₂₄O₅: C, 71.72; H, 6.57. Found: C, 71.52; H, 6.22.

### Example 7

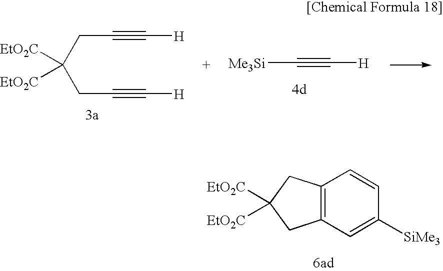

The substituted benzene 6ad was obtained from the compound 3a and compound 4d in a similar manner as in Example 4 except for the use of the compound 4d (3 mmol) and stirring at room temperature for 2 hours (yield: 83%).

¹H-NMR (600 MHz, CDCl₃): δ 7.37 (s, 1H, Ar), 7.34 (d, 1H, J=7.2 Hz, Ar), 7.21 (d, 1H, J=7.2 Hz, Ar), 4.22 (q, 4H, J=7.2 Hz, OCH₂CH₃), 3.62 (s, 2H, ArCH₂C), 3.61 (s, 2H, ArCH₂C), 1.27 (t, 6H, J=7.2 Hz, OCH₂CH₃), 0.27 (s, 9H, SiMe₃).

¹³C-NMR (150 MHz, CDCl₃): δ 171.7, 140.8, 139.4, 138.8, 132.0, 129.0, 123.6, 61.6, 60.1, 40.5, 40.4, 14.0, −1.0.

IR (neat): 2980, 2957, 1728, 1242, 1194, 1180 cm⁻¹.

Anal. Calcd for C₁₈H₂₆O₄Si: C, 64.64; H, 7.83. Found: C, 64.71; H, 7.52.

### Example 8

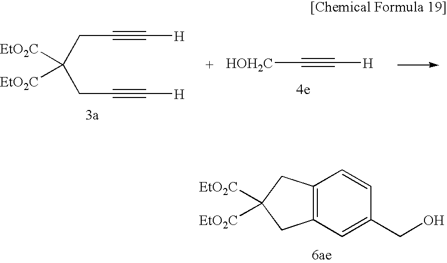

The substituted benzene 6ae was obtained from the compound 3a and compound 4e in a similar manner as in Example 4 except for the use of the compound 4e (3 mmol) (yield: 96%).

¹H-NMR (600 MHz, CDCl₃): δ 7.20 (s, 1H, Ar), 7.17 (d, 1H, J=8.4 Hz, Ar), 7.14 (d, 1H, J=8.4 Hz, Ar), 4.62 (s, 2H, ArCH₂OH), 4.20 (q, 4H, J=6.6 Hz, OCH₂CH₃), 3.57 (s, 4H, ArCH₂C), 1.25 (t, 6H, J=6.6 Hz, OCH₂CH₃).

¹³C-NMR (150 MHz, CDCl₃): δ 171.6, 140.4, 139.8, 139.5, 125.9, 124.2, 123.0, 65.3, 61.7, 60.5, 40.3, 40.1, 14.0.

IR (neat): 3550, 2984, 2938, 1753, 1735, 1727, 1712, 1258, 1186, 1157 cm⁻¹.

Anal. Calcd for C₁₆H₂₀O₅: C, 65.74; H, 6.90. Found: C, 65.40; H, 6.95.

### Example 9

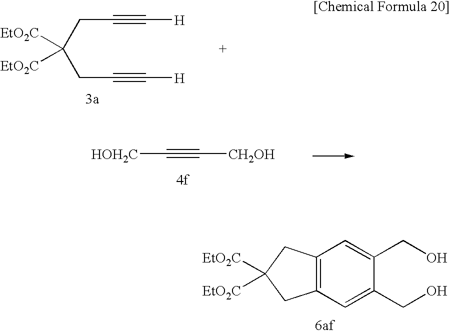

The substituted benzene 6af was obtained from the compound 3a and compound 4f in a similar manner as in Example 4 except for the use of the compound 4f (3 mmol) and stirring at room temperature for 8 hours (yield: 99%).

¹H-NMR (600 MHz, CDCl₃): δ 7.17 (s, 2H, Ar), 4.66 (s, 4H, ArCH₂OH), 4.20 (q, 4H, J=7.2 Hz, OCH₂CH₃), 3.56 (s, 4H, ArCH₂C), 1.25 (t, 6H, J=7.2 Hz, OCH₂CH₃).

¹³C-NMR (150 MHz, CDCl₃): δ 171.6, 140.4, 138.5, 125.6, 64.2, 61.8, 60.5, 40.2, 14.0.

IR (neat): 3320, 2980, 1728, 1246, 1192 cm⁻¹.

Anal. Calcd for C₁₇H₂₂O₆: C, 63.34; H, 6.88. Found: C, 63.04; H, 7.22.

### Example 10

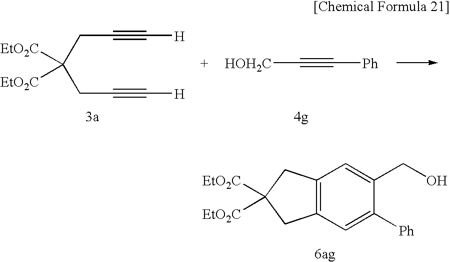

The substituted benzene 6ag was obtained from the compound 3a and compound 4g in a similar manner as in Example 4 except for the use of the compound 4g (3 mmol) (yield: 91%).

¹H-NMR (500 MHz, CDCl₃): δ 7.31-7.42 (m, 6H, Ar), 7.10 (s, 1H, Ar), 4.55 (s, 2H, ArCH₂OH), 4.22 (q, 4H, J=7.0 Hz, OCH₂CH₃), 3.64 (s, 2H, ArCH₂C), 3.61 (s, 2H, ArCH₂C), 1.27 (t, 6H, J=7.0 Hz, OCH₂CH₃).

¹³C-NMR (125 MHz, CDCl₃): δ 171.6, 140.8, 140.3, 139.6, 137.0, 129.1 (3C), 128.2, 127.1, 125.8, 124.2, 63.1, 61.7, 60.5, 40.3, 40.2, 14.0.

IR (neat): 3455, 2936, 1723, 1601, 1242, 1186, 1153 cm⁻¹.

Anal. Calcd for C₂₂H₂₄O₅: C, 71.72; H, 6.57. Found: C, 71.65; H, 6.23.

### Example 11

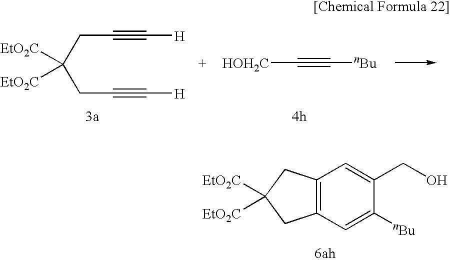

The substituted benzene 6ah was obtained from the compound 3a and compound 4h in a similar manner as in Example 4 except for the use of the compound 4h (3 mmol) and stirring at room temperature for 12 hours (yield: 80%).

¹H-NMR (600 MHz, CDCl₃): δ 7.20 (s, 1H, Ar), 7.03 (s, 1H, Ar), 4.66 (s, 2H, ArCH₂OH), 4.19 (q, 4H, J=7.2 Hz, OCH₂CH₃), 3.55 (s, 2H, ArCH₂C), 3.54 (s, 2H, ArCH₂C), 2.62 (t, 2H, J=7.8 Hz, ArCH₂CH₂), 1.54 (quint, 2H, J=7.2 Hz, CH₂CH₂CH₃), 1.39 (sext, 2H, J=7.2 Hz, CH₂CH₂CH₃), 1.25 (t, 6H, J=7.2 Hz, OCH₂CH₃), 0.94 (t, 3H, J=7.2 Hz, CH₂CH₂CH₃).

¹³C-NMR (150 MHz, CDCl₃): δ 171.7, 139.8, 139.7, 137.7, 137.1, 125.0, 123.9, 63.1, 61.6, 60.5, 40.3, 40.2, 33.6, 32.0, 22.7, 13.97, 13.95.

IR (neat): 3281, 2980, 2963, 1732, 1242, 1184 cm⁻¹.

Anal. Calcd for C₂₀H₂₈O₅: C, 68.94; H, 8.10. Found: C, 68.66; H, 7.74.

### Example 12

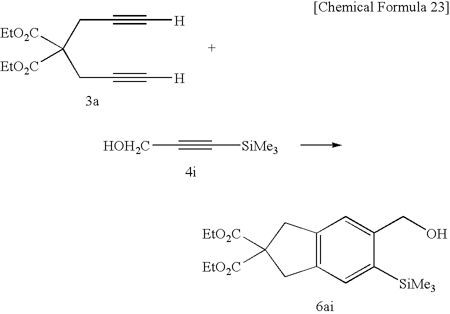

The substituted benzene 6ai was obtained from the compound 3a and compound 4i in a similar manner as in Example 4 except for the use of the compound 4i (3 mmol) and stirring at room temperature for 12 hours (yield: 98%).

¹H-NMR (600 MHz, CDCl₃): δ 7.35 (s, 1H, Ar), 7.32 (s, 1H, Ar), 4.74 (s, 2H, ArCH₂OH), 4.20 (q, 4H, J=7.2 Hz, OCH₂CH₃), 3.59 (s, 2H, ArCH₂C), 3.58 (s, 2H, ArCH₂C), 1.26 (t, 6H, J=7.2 Hz, OCH₂CH₃), 0.32 (s, 9H, SiMe₃).

¹³C-NMR (150 MHz, CDCl₃): δ 171.7, 145.2, 141.7, 138.8, 136.6, 130.4, 123.9, 65.2, 61.7, 60.2, 40.5, 40.3, 14.0, 0.4.

IR (neat): 3458, 2953, 2899, 1726, 1250, 1192 cm⁻¹.

Anal. Calcd for C₁₉H₂₈O₅Si: C, 62.61; H, 7.74. Found: C, 62.85; H, 7.46.

### Example 13

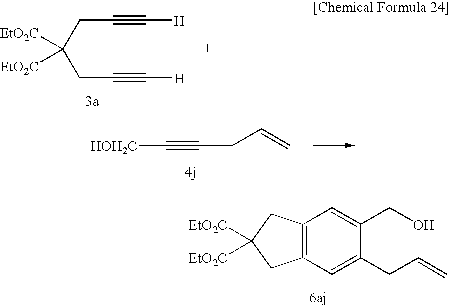

The substituted benzene 6aj was obtained from the compound 3a and compound 4j in a similar manner as in Example 4 except for the use of the compound 4j (3 mmol) and stirring at room temperature for 8 hours (yield: 73%).

¹H-NMR (600 MHz, CDCl₃): δ 7.22 (s, 1H, Ar), 7.03 (s, 1H, Ar), 5.94-6.01 (m, 1H, CH₂CH═CH₂), 5.06 (d, 1H, J=9.6 Hz, CH₂CH═CH₂), 5.00 (d, 1H, J=16.8 Hz, CH₂CH═CH₂), 4.65 (s, 2H, ArCH₂OH), 4.20 (q, 4H, J=7.2 Hz, OCH₂CH₃), 3.56 (s, 4H, cyclic ArCH₂C), 3.42 (d, 2H, J=6.6 Hz, acyclic ArCH₂C), 1.55-1.70 (br, 1H, OH), 1.25 (t, 6H, J=7.2 Hz, OCH₂CH₃).

¹³C-NMR (150 MHz, CDCl₃): δ 171.6, 140.0, 138.4, 137.6, 137.5, 136.7, 125.6, 124.3, 115.8, 63.2, 61.7, 60.5, 40.3, 40.2, 36.8, 14.0.

IR (neat): 3412, 3078, 2980, 2931, 2906, 1724, 1246, 1157 cm⁻¹.

Anal. Calcd for C₁₉H₂₄O₅: C, 68.66; H, 7.28. Found: C, 68.98; H, 6.93.

### Example 14

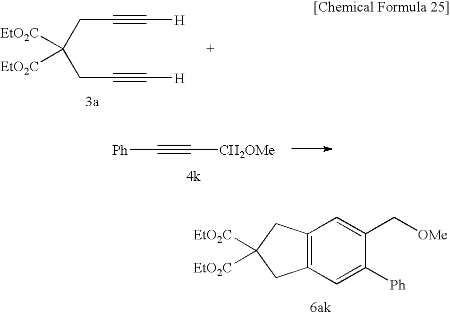

The substituted benzene 6ak was obtained from the compound 3a and compound 4k in a similar manner as in Example 4 except for the use of the compound 4k (3 mmol) and stirring at room temperature for 8 hours (yield: 87%).

¹H-NMR (600 MHz, CDCl₃): δ 7.32-7.41 (m, 6H, Ar), 7.11 (s, 1H, Ar), 4.28 (s, 2H, ArCH₂O), 4.22 (q, 4H, J=7.2 Hz, OCH₂CH₃), 3.64 (s, 2H, ArCH₂C), 3.61 (s, 2H, ArCH₂C), 3.33 (s, 3H, OMe), 1.26 (t, 6H, J=7.2 Hz, OCH₂CH₃).

¹³C-NMR (150 MHz, CDCl₃): δ 171.8, 141.0, 139.8, 139.6, 134.5, 129.5 (3C), 128.2, 127.2, 125.9, 125.0, 72.7, 61.9, 60.7, 58.3, 40.5 (2C), 14.2.

IR (neat): 2980, 2929, 1726, 1244, 1155, 1097 cm⁻¹.

Anal. Calcd for C₂₃H₂₈O₅: C, 72.23; H, 6.85. Found: C, 72.00; H, 6.48.

### Example 15

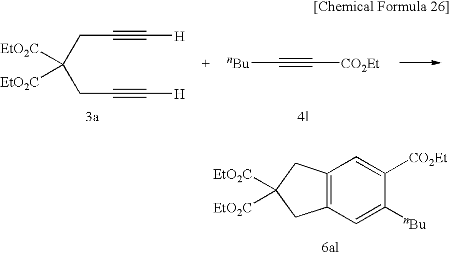

The substituted benzene 6al was obtained from the compound 3a and compound 4l in a similar manner as in Example 4 except for the use of the compound 4l (3 mmol) and stirring at room temperature for 8 hours (yield: 94%).

¹H-NMR (600 MHz, CDCl₃): δ 7.68 (s, 1H, Ar), 7.07 (s, 1H, Ar), 4.34 (q, 2H, J=7.2 Hz, OCH₂CH₃), 4.20 (q, 4H, J=7.2 Hz, OCH₂CH₃), 3.58 (s, 4H, ArCH₂C), 2.89 (t, 2H, J=7.8 Hz, ArCH₂CH₂), 1.55 (quint, 2H, J=7.8 Hz, CH₂CH₂CH₃), 1.37 (t, 3H, J=7.2 Hz, OCH₂CH₃), 1.34-1.42 (m, 2H, CH₂CH₂CH₃), 1.25 (t, 6H, J=7.2 Hz, OCH₂CH₃), 0.92 (t, 3H, J=7.8 Hz, CH₂CH₂CH₃).

¹³C-NMR (150 MHz, CDCl₃): δ 171.3, 167.8, 144.2, 143.6, 137.5, 128.7, 126.5, 126.1, 61.7, 60.6, 60.4, 40.4, 39.9, 34.2, 34.1, 22.8, 14.2, 14.0, 13.9.

IR (neat): 2960, 2936, 2872, 1738, 1726, 1713, 1246, 1157 cm⁻¹.

Anal. Calcd for C₂₂H₃₀O₆: C, 67.67; H, 7.74. Found: C, 67.74; H, 7.41.

### Example 16

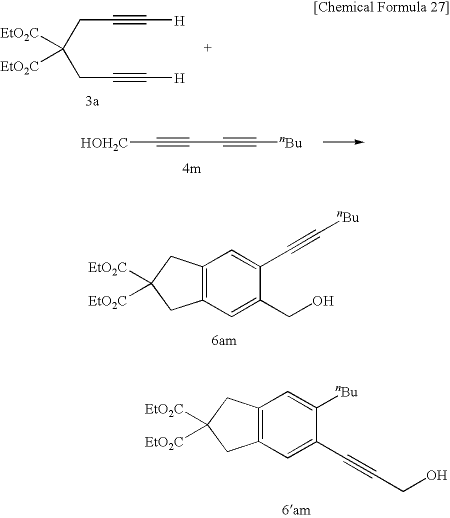

The substituted benzenes 6am and 6′am were obtained from the compound 3a and compound 4m in a similar manner as in Example 4 except for stirring at room temperature for 24 hours (6am:6′am=87:13, total yield: 85%).

6am:

¹H-NMR (500 MHz, CDCl₃): δ 7.24 (s, 1H, Ar), 7.22 (s, 1H, Ar), 4.74 (d, 2H, J=6.5 Hz, ArCH₂OH), 4.19 (q, 4H, J=7.0 Hz, OCH₂CH₃), 3.56 (s, 2H, ArCH₂C), 3.53 (s, 2H, ArCH₂C), 2.43 (t, 2H, J=7.5 Hz, C≡CCH₂), 1.59 (quint, 2H, J=7.5 Hz, CH₂CH₂CH₃), 1.48 (sext, 2H, J=7.5 Hz, CH₂CH₂CH₃), 1.25 (t, 6H, J=7.0 Hz, OCH₂CH₃), 0.95 (t, 3H, J=7.5 Hz, CH₂CH₂CH₃).

¹³C-NMR (125 MHz, CDCl₃): δ 171.3, 141.5, 140.1, 139.1, 127.7, 123.0, 120.3, 94.6, 78.2, 63.9, 61.7, 60.4, 40.3, 39.9, 30.8, 21.9, 19.1, 13.9, 13.5.

6′am (Selected Peaks):

¹H-NMR (500 MHz, CDCl₃): δ 7.02 (s, 1H, Ar), 4.50 (d, 2H, J=6.0 Hz, ArCH₂OH), 2.71 (t, 2H, J=7.5 Hz, C≡CCH₂).

IR (neat): 3495, 2959, 1730, 1244, 1186 cm⁻¹. (measured on the mixture of 6am and 6′am)

Anal. Calcd for C₂₂H₂₈O₅: C, 70.94; H, 7.58. Found: C, 70.96; H, 7.46. (measured on the mixture of 6am and 6′am)

### Example 17

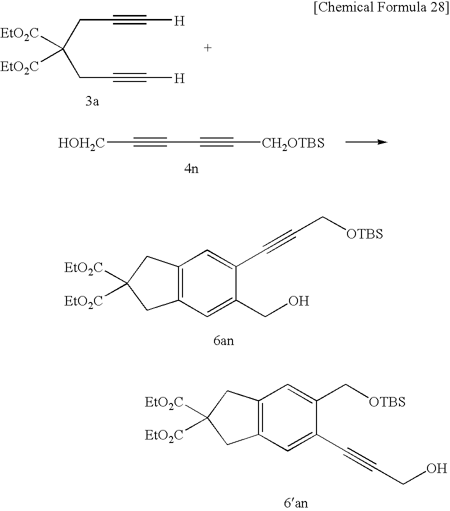

The substituted benzenes 6an and 6′an were obtained from the compound 3a and compound 4n in a similar manner as in Example 4 except for stirring at room temperature for 24 hours (6an:6′an=82:18, total yield: 94%).

6an:

¹H-NMR (500 MHz, CDCl₃): δ 7.35 (s, 1H, Ar), 7.22 (s, 1H, Ar), 4.81 (s, 2H, ArCH₂OH), 4.49 (d, 2H, J=5.2 Hz, CH₂OTBS), 4.20 (q, 4H, J=6.9 Hz, OCH₂CH₃), 3.59 (s, 2H, ArCH₂C), 3.53 (s, 2H, ArCH₂C), 1.26 (t, 6H, J=6.9 Hz, OCH₂CH₃), 0.95 (s, 9H, t-Bu), 0.11 (s, 6H, Si(Me)₂).

¹³C-NMR (125 MHz, CDCl₃): δ 171.4, 142.2, 141.1, 138.3, 127.6, 121.9, 118.0, 83.2, 63.2, 61.7, 60.4, 51.6, 40.6, 39.9, 25.9, 14.0, −5.3.

6′an (Selected Peaks):

¹H-NMR (500 MHz, CDCl₃): δ 7.27 (s, 1H, Ar), 4.76 (s, 2H, ArCH₂OTBS), 4.55 (s, 2H, CCH₂OH), 3.57 (s, 2H, ArCH₂C), 3.54 (s, 2H, ArCH₂C), 0.93 (s, 9H, t-Bu), 0.16 (s, 6H, Si(Me)₂).

¹³C-NMR (125 MHz, CDCl₃): δ 52.2, 31.5, 25.8, 22.6, 18.4, 14.1, −5.1.

IR (neat): 3462, 2930, 1732, 1248, 1070 cm⁻¹. (measured on the mixture of 6an and 6′an)

Anal. Calcd for C₂₅H₃₆O₆Si: C, 65.19; H, 7.88. Found: C, 64.99; H, 7.91. (measured on the mixture of 6an and 6′an)

### Example 18

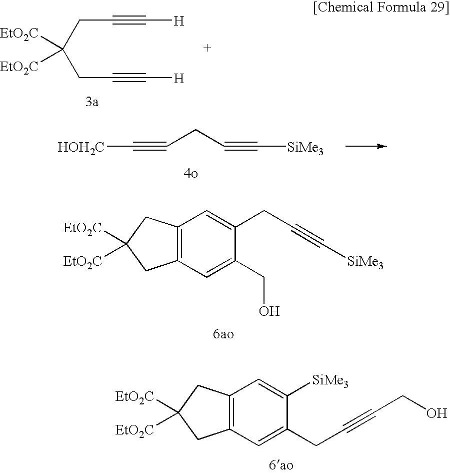

The substituted benzenes 6ao and 6′ao were obtained from the compound 3a and compound 4o in a similar manner as in Example 4 except for stirring at room temperature for 24 hours (6ao:6′ao=82:18, total yield: 80%).

6ao:

¹H-NMR (500 MHz, CDCl₃): δ 7.24 (s, 1H, Ar), 7.20 (s, 1H, Ar), 4.66 (s, 2H, ArCH₂OH), 4.19 (q, 4H, J=7.0 Hz, OCH₂CH₃), 3.63 (s, 2H, ArCH₂C≡C), 3.57 (s, 2H, cyclic ArCH₂C), 3.55 (s, 2H, cyclic ArCH₂C), 1.25 (t, 6H, J=7.0 Hz, OCH₂CH₃), 0.16 (s, 9H, SiMe₃).

¹³C-NMR (125 MHz, CDCl₃): δ 171.6, 140.1, 139.1, 137.3, 133.6, 125.0, 124.6, 104.8, 87.3, 63.2. 61.8, 60.5, 40.3, 40.2, 23.7, 14.0, 0.0.

6′ao (Selected Peaks):

¹H-NMR (500 MHz, CDCl₃): δ 7.35 (s, 1H, Ar), 7.29 (s, 1H, Ar), 4.27 (s, 2H, CCH₂OH), 3.67 (s, 2H, acyclic ArCH₂C), 3.58 (s, 2H, cyclic ArCH₂C), 0.31 (s, 9H, SiMe₃).

¹³C-NMR (125 MHz, CDCl₃): δ 171.7, 130.2, 40.5, 25.6, 0.2.

IR (neat): 3447, 2959, 2174, 1734, 1250, 845 cm⁻¹. (measured on the mixture of 6ao and 6′ao)

Anal. Calcd for C₂₂H₃₀O₅Si: C, 65.64; H, 7.51. Found: C, 65.47; H, 7.58. (measured on the mixture of 6ao and 6′ao)

### Example 19

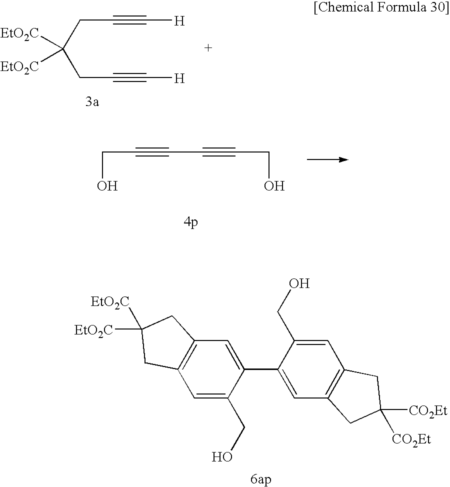

The substituted benzene 6ap was obtained in a similar manner as in Example 4 except for the use of the compound 3a (2.2 mmol) and the compound 4p (1 mmol) and stirring at room temperature for 24 hours (yield: 81%).

¹H-NMR (500 MHz, CDCl₃): δ 7.27 (s, 2H, Ar), 6.91 (s, 2H, Ar), 4.19-4.28 (m, 12H, ArCH₂OH and OCH₂CH₃), 3.65 and 3.61 (2d, each 2H, J=16.6 Hz, ArCH₂C), 3.60 and 3.55 (2d, each 2H, J=16.6 Hz, ArCH₂C), 3.16 (s, 2H, OH), 1.27 (t, 6H, J=6.9 Hz, OCH₂CH₃), 1.26 (t, 6H, J=6.9 Hz, OCH₂CH₃).

¹³C-NMR (125 MHz, CDCl₃): δ 171.6, 171.5, 139.8, 139.5, 139.0, 137.7, 125.4, 125.2, 62.6, 61.75, 61.71, 60.4, 40.22, 40.18, 14.0.

IR (neat): 3374, 2980, 1726, 1445, 1246 cm⁻¹.

Anal. Calcd for C₃₂H₃₈O₁₀: C, 65.97; H, 6.57. Found: C, 65.58; H, 6.53.

### Example 20

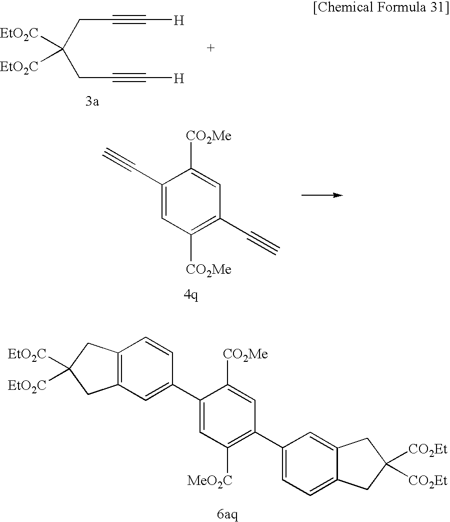

The substituted benzene 6aq was obtained in a similar manner as in Example 4 except for the use of the compound 3a (2 mmol) and the compound 4q (1 mmol) and stirring at room temperature for 8 hours (yield: 79%).

¹H-NMR (500 MHz, CDCl₃): δ 7.77 (s, 2H, Ar), 7.23 (d, 2H, J=7.5 Hz, Ar), 7.20 (s, 2H, Ar), 7.13 (d, 2H, J=7.5 Hz, Ar), 4.23 (q, 8H, J=7.0 Hz, OCH₂CH₃), 3.67 (s, 6H, OMe), 3.64 (s, 8H, ArCH₂C), 1.28 (t, 12H, J=7.0 Hz, OCH₂CH₃).

¹³C-NMR (125 MHz, CDCl₃): δ 171.6, 168.4, 140.9, 140.3, 139.6, 138.7, 133.1, 131.7, 127.2, 124.1, 123.9, 61.7, 60.4, 52.2, 40.4, 40.3, 14.0.

IR (KBr): 2988, 1740, 1719, 1269, 1223, 1184 cm⁻¹.

Mp: 166-167° C.

Anal. Calcd for C₄₀H₄₂O₁₂: C, 67.22; H, 5.92. Found: C, 67.18; H, 6.20.

### Example 21

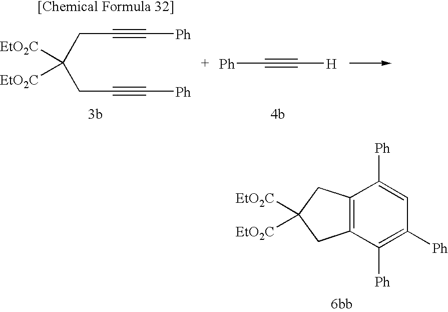

The substituted benzene 6bb was obtained from the compound 3b and compound 4b in a similar manner as in Example 4 except for stirring at room temperature for 8 hours (yield: 98%).

¹H-NMR (500 MHz, CDCl₃): δ 7.07-7.55 (m, 16H, Ar), 4.16 (q, 4H, J=7.0 Hz, OCH₂CH₃), 3.76 (s, 2H, ArCH₂C), 3.51 (s, 2H, ArCH₂C), 1.20 (t, 6H, J=7.0 Hz, OCH₂CH₃).

¹³C-NMR (125 MHz, CDCl₃, two carbons overlap): δ 171.5, 141.1, 140.5, 140.1, 139.3, 137.3, 136.9, 136.0, 130.1, 130.0, 129.9, 128.5, 128.4, 127.9, 127.6, 127.2, 126.5, 126.2, 61.7, 60.3, 40.6, 40.4, 14.0.

IR (KBr): 2980, 2928, 1728, 1601, 1460, 1443, 1273, 1196 cm⁻¹.

Mp: 154-156° C.

Anal. Calcd for C₃₃H₃₀O₄: C, 80.79; H, 6.16. Found: C, 80.90; H, 6.32.

### Example 22

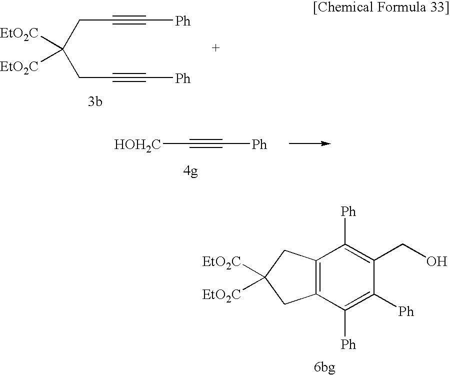

The substituted benzene 6bg was obtained from the compound 3b and compound 4g in a similar manner as in Example 4 except for stirring at room temperature for 8 hours (yield: 95%).

¹H-NMR (500 MHz, CDCl₃): δ 7.02-7.50 (m, 15H, Ph), 4.22 (d, 2H, J=6.6 Hz, ArCH₂OH), 4.15 (q, 4H, J=7.2 Hz, OCH₂CH₃), 3.44 (s, 2H, ArCH₂C), 3.41 (s, 2H, ArCH₂C), 1.19 (t, 6H, J=7.2 Hz, OCH₂CH₃).

¹³C-NMR (125 MHz, CDCl₃): δ 171.5, 141.0, 139.5, 139.3, 139.1, 138.5, 138.2, 137.7, 135.6, 130.5, 129.6, 129.2, 128.5, 127.6, 127.5, 127.3, 126.5, 126.3, 61.6, 60.2, 59.8, 40.8, 40.6, 13.9.

IR (KBr): 3553, 2976, 2940, 2887, 1726, 1248, 1159 cm⁻¹.

Mp: 213-214° C.

Anal. Calcd for CO₃₂O₅: C, 78.44; H, 6.20. Found: C, 78.31; H, 6.11.

### Example 23

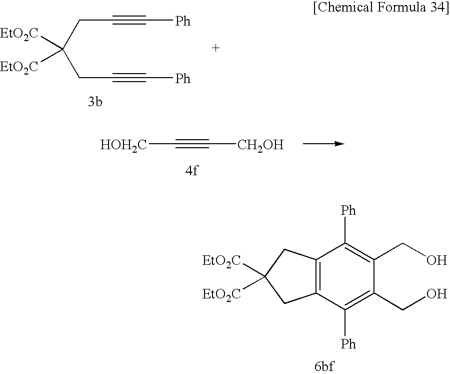

The substituted benzene 6bf was obtained from the compound 3b and compound 4f in a similar manner as in Example 4 except for stirring at room temperature for 8 hours (yield: 90%)

¹H-NMR (600 MHz, CDCl₃): δ 7.36-7.45 (m, 6H, Ar), 7.31 (d, 4H, J=8.3 Hz, Ar), 4.54 (s, 4H, ArCH₂OH), 4.11 (q, 4H, J=7.2 Hz, OCH₂CH₃), 3.36 (s, 4H, ArCH₂C), 3.25-3.35 (br, 2H, OH), 1.17 (t, 6H, J=7.2 Hz, OCH₂CH₃).

¹³C-NMR (150 MHz, CDCl₃): δ 171.4, 138.9, 138.8, 138.6, 137.1, 129.1, 128.4, 127.3, 61.6, 60.3, 59.7, 40.7, 13.9.

IR (KBr): 3312, 2980, 2936, 1728, 1260, 1184 cm⁻¹.

Mp: 168-169° C.

Anal. Calcd for C₂₉H₃₀O₆: C, 73.40; H, 6.37. Found: C, 73.23; H, 6.31.

### Example 24

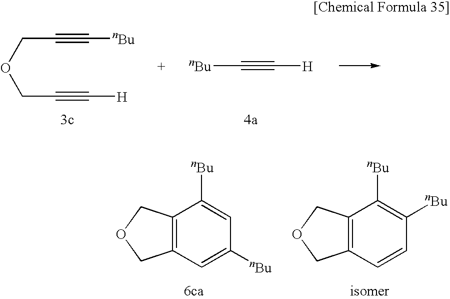

The substituted benzene 6ca was obtained from the compound 3c and compound 4a in a similar manner as in Example 4 except for the use of the compound 4a (3 mmol) (6ca:isomer=76:24, total yield: 52%).

6ca:

¹H-NMR (600 MHz, CDCl₃): δ 6.87 (s, 2H Ar), 5.08 (s, 4H, ArCH₂O), 2.59 (t, 2H, J=7.8 Hz, ArCH₂C), 2.49 (t, 2H, J=7.8 Hz, ArCH₂C), 1.52-1.61 (m, 4H, CH₂CH₂CH₃) 1.36 (sext, 4H, J=7.8 Hz, CH₂CH₂CH₃), 0.93 (t, 6H, J=7.8 Hz, CH₂CH₂CH₃).

¹³C-NMR (150 MHz, CDCl₃): δ 142.6, 139.1, 136.0, 134.9, 127.5, 118.1, 73.9, 72.7, 35.5, 34.0, 33.2, 32.2, 22.4 (2C), 14.0, 13.9.

Isomer (Selected Peaks):

¹H-NMR (600 MHz, CDCl₃): δ 7.07 (d, 1H, J=7.2 Hz, Ar), 6.98 (d, 1H, J=7.2 Hz, Ar).

IR (neat): 2950, 2925, 2859, 1055 cm⁻¹. (measured on the mixture of 6ca and the isomer)

Anal. Calcd. for C₁₆H₂₄O: C, 82.70; H, 10.41. Found: C, 82.33; H, 10.29. (measured on the mixture of 6ca and the isomer)

### Example 25

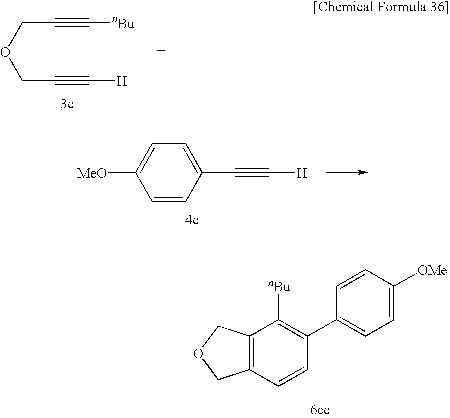

The substituted benzene 6cc was obtained from the compound 3c and compound 4c in a similar manner as in Example 4 except for stirring at room temperature for 2 hours (yield: 48%, 6cc:isomer=99:1).

¹H-NMR (600 MHz, CDCl₃): δ 7.21 (d, 2H, J=9.0 Hz, Ar), 7.12 (d, 1H, J=7.2 Hz, Ar), 7.08 (d, 1H, J=7.2 Hz, Ar), 6.94 (d, 2H, J=9.0 Hz, Ar), 5.17 (s, 4H, ArCH₂O), 3.86 (s, 3H, OMe), 2.47 (t, 2H, J=7.8 Hz, ArCH₂C), 1.33 (quint, 2H, J=7.8 Hz, CH₂CH₂CH₃), 1.19 (sext, 2H, J=7.8 Hz, CH₂CH₂CH₃), 0.77 (t, 3H, J=7.8 Hz, CH₂CH₂CH₃).

¹³C-NMR (150 MHz, CDCl₃): δ 158.5, 140.7, 138.4, 137.9, 134.3, 133.9, 130.3, 129.9, 117.9, 113.4, 74.0, 73.1, 55.3, 32.1, 30.5, 22.7, 13.7.

IR (KBr): 2953, 2926, 2860, 1607, 1510, 1468, 1238, 1107, 1032 cm⁻¹.

Mp: 79-81° C.

Anal. Calcd for C₁₉H₂₂O₂: C, 80.82; H, 7.85. Found: C, 80.97; H, 7.54.

### Example 26

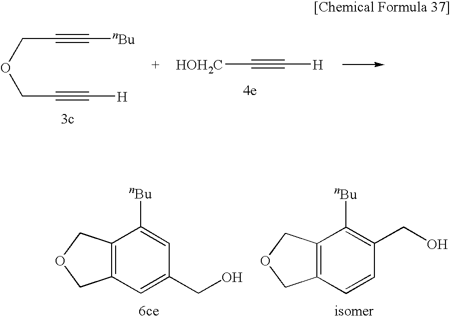

The substituted benzene 6ce was obtained from the compound 3c and compound 4e in a similar manner as in Example 4 except for the use of the compound 4e (3 mmol) (6ce:isomer=71:29, total yield: 74%).

6ce:

¹H-NMR (600 MHz, CDCl₃): δ 7.05 (s, 2H, Ar), 5.08 (s, 2H, ArCH₂O), 5.07 (s, 2H, ArCH₂O), 4.66 (s, 2H, ArCH₂OH), 2.50 (t, 2H, J=7.8 Hz, ArCH₂C), 1.56 (quint, 2H, J=7.8 Hz, CH₂CH₂CH₃), 1.36 (sext, 2H, J=7.8 Hz, CH₂CH₂CH₃), 0.93 (t, 3H, J=7.8 Hz, CH₂CH₂CH₃).

¹³C-NMR (150 MHz, CDCl₃): δ 140.8, 136.4, 128.2, 126.1, 118.4, 116.9, 73.7, 72.6, 65.2, 33.1, 32.2, 22.5, 13.9.

Isomer (Selected Peaks):

¹H-NMR (600 MHz, CDCl₃): δ 7.29 (d, 1H, J=7.8 Hz, Ar), 4.71 (s, 2H, ArCH₂OH), 2.56 (t, 2H, J=7.8 Hz, ArCH₂C).

IR (neat): 3454, 2935, 2850, 1053 cm⁻¹. (measured on the mixture of 6ce and the isomer).

Anal. Calcd. for C₁₃H₁₈O₂: C, 75.69; H, 8.80. Found: C, 75.53; H, 8.84. (measured on the mixture of 6ce and the isomer).

### Example 27

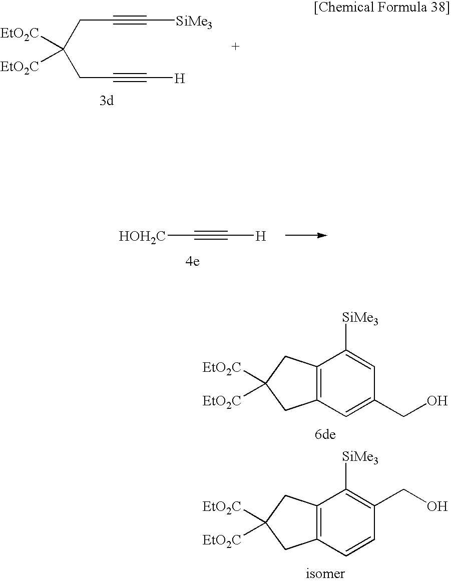

The substituted benzene 6de was obtained from the compound 3d and compound 4e in a similar manner as in Example 4 except for the use of the compound 4e (3 mmol) and stirring at room temperature for 8 hours (6de:isomer=85:15, total yield: 85%).

6de:

¹H-NMR (600 MHz, CDCl₃): δ 7.28 (s, 1H, Ar), 7.22 (s, 1H, Ar), 4.64 (s, 2H, ArCH₂OH), 4.20 (q, 4H, J=7.2 Hz, OCH₂CH₃), 3.60 (s, 2H, ArCH₂C), 3.56 (s, 2H, ArCH₂C), 1.58-1.72 (br, 1H, OH), 1.25 (t, 6H, J=7.2 Hz, OCH₂CH₃), 0.31 (s, 9H, SiMe₃).

¹³C-NMR (150 MHz, CDCl₃): δ 171.6, 144.9, 139.8, 138.8, 135.8, 131.5, 124.1, 65.6, 61.7, 60.7, 41.1, 39.9, 14.0, −0.9.

Isomer (Selected Peaks):

¹H-NMR (600 MHz, CDCl₃): δ 7.22 (d, 1H, J=8.1 Hz, Ar), 7.19 (d, 1H, J=8.1 Hz, Ar), 4.70 (s, 2H, ArCH₂OH), 4.19 (q, 4H, J=7.4 Hz, OCH₂CH₃), 3.65 (s, 2H, ArCH₂C), 3.52 (s, 2H, ArCH₂C), 1,25 (t, 6H, J=7.4 Hz, OCH₂CH₃), 0.42 (s, 9H, SiMe₃).

IR (neat): 3416, 2961, 2907, 1726, 1238, 1153 cm⁻¹. (measured on the mixture of 6de and the isomer).

Anal. Calcd for C₁₉H₂₈O₅Si: C, 62.61; H, 7.74. Found: C, 62.48; H, 7.43. (measured on the mixture of 6de and the isomer).

### Example 28

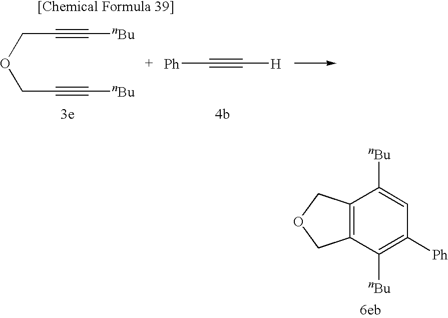

The substituted benzene 6eb was obtained from the compound 3e and compound 4b in a similar manner as in Example 4 except for stirring at room temperature for 24 hours (yield: 75%).

¹H-NMR (600 MHz, CDCl₃): δ 7.27-7.40 (m, 5H, Ph), 6.94 (s, 1H, Ar), 5.18 (s, 2H, ArCH₂O), 5.17 (s, 2H, ArCH₂O), 2.50 (t, 2H, J=7.8 Hz, ArCH₂C), 2.44 (t, 2H, J=7.8 Hz, ArCH₂C), 1.57 (quint, 2H, J=7.8 Hz, CH₂CH₂CH₃), 1.37 (sext, 2H, J=7.8 Hz, CH₂CH₂CH₃), 1.32 (quint, 2H, J=7.8 Hz, CH₂CH₂CH₃), 1.16 (sext, 2H, J=7.8 Hz, CH₂CH₂CH₃), 0.92 (t, 3H, J=7.8 Hz, CH₂CH₂CH₃), 0.74 (t, 3H, J=7.8 Hz, CH₂CH₂CH₃).

¹³C-NMR (150 MHz, CDCl₃): δ 141.7, 141.5, 138.2, 136.7, 133.2, 131.3, 129.6, 129.3, 127.9, 126.7, 73.5, 73.2, 32.8, 32.2, 32.1, 30.1, 22.7, 22.5, 13.9, 13.6.

IR (neat): 2957, 2930, 2859, 1601, 1500, 1483, 1103, 1059 cm⁻¹.

Anal. Calcd for C₂₂H₂₈O: C, 85.66; H, 9.15. Found: C, 85.55; H, 9.18.

### Example 29

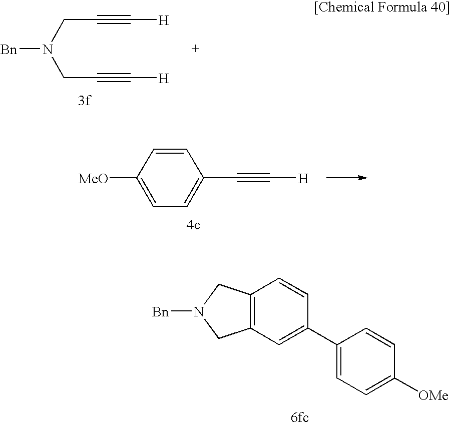

The substituted benzene 6fc was obtained from the compound 3f and compound 4c in a similar manner as in Example 4 except for stirring at room temperature for 2 hours (yield: 81%)

¹H-NMR (600 MHz, CDCl₃): δ 7.48 (d, 2H, J=8.4 Hz, Ar), 7.43 (d, 2H, J=7.2 Hz, Ar), 7.33-7.37 (m, 4H, Ar), 7.29 (t, 1H, J=7.2 Hz, Ar), 7.21 (d, 1H, J=7.2 Hz, Ar), 6.96 (d, 2H, J=8.4 Hz, Ar), 3.98 (s, 2H, cyclic ArCH₂N), 3.96 (s, 2H, cyclic ArCH₂N), 3.94 (s, 2H, PhCH₂N), 3.84 (s, 3H, OMe).

¹³C-NMR (150 MHz, CDCl₃): δ 159.0, 140.9, 139.8, 139.0, 138.7, 134.0, 128.8, 128.4, 128.1, 127.1, 125.5, 122.5, 120.8, 114.1, 60.3, 59.0, 58.7, 55.3.

IR (KBr): 2968, 2938, 1637, 1609, 1518, 1244, 1179, 1036 cm⁻¹.

Mp: 140-141° C.

Anal. Calcd. for C₂₂H₂₁NO: C, 83.78; H, 6.71; N, 4.44. Found: C, 83.46; H, 6.54; N, 4.23.

### Example 30

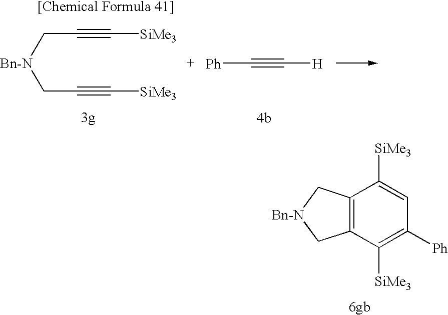

The substituted benzene 6gb was obtained from the compound 3g and compound 4b in a similar manner as in Example 4 except for stirring at room temperature for 2 hours (yield: 98%)

¹H-NMR (600 MHz, CDCl₃): δ 7.22-7.46 (m, 10H, Ph), 7.16 (s, 1H, Ar), 4.00 (s, 2H, cyclic ArCH₂N), 3.96 (s, 2H, cyclic ArCH₂N), 3.95 (s, 2H, acyclic PhCH₂N), 0.24 (s, 9H, SiMe₃), −0.08 (s, 9H, SiMe₃).

¹³C-NMR (150 MHz, CDCl₃): δ 147.3, 145.42, 145.40, 143.5, 138.9, 134.1, 133.8, 133.0, 129.6, 128.7, 128.4, 127.7, 127.1, 126.9, 60.7, 60.5, 59.1, 1.4, −0.9.

IR (KBr): 2949, 1337, 1246, 1123 cm⁻¹.

Mp: 171-173° C.

Anal. Calcd for C₂₇H₃₅NSi₂: C, 75.46; H, 8.21; N, 3.26. Found: C, 75.07; H, 8.21; N, 2.90.

### Example 31

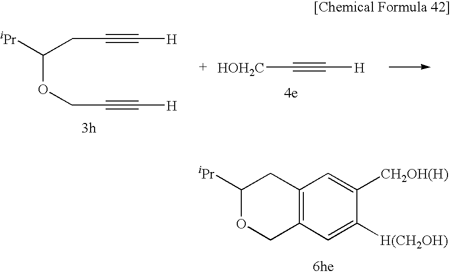

The substituted benzene 6he was obtained from the compound 3h and compound 4e in a similar manner as in Example 4 except for the use of the compound 4e (3 mmol) and stirring at room temperature for 24 hours (1:1 mixture of positional isomers, total yield: 68%).

¹H-NMR (600 MHz, CDCl₃): δ 7.08-7.15 (m, 2H, Ar), 6.96-7.00 (m, 1H, Ar), 4.80-4.86 (m, 1H, ArCH₂O), 4.73-4.76 (m, 1H, ArCH₂O), 3.29-3.34 (m, 1H, ArCH₂CHO), 2.67-2.75 (m, 2H, ArCH₂CHO), 1.85-1.95 (br, 1H, OH), 1.81 (m, 1H, CH(CH₃)₂), 1.04 (d, 3H, J=7.2 Hz, CH(CH₃)₂), 0.99 (d, 3H, J=7.2 Hz, CH(CH₃)₂).

¹³C-NMR (150 MHz, CDCl₃): δ 139.0, 138.5, 135.2, 134.4, 134.0, 133.2, 129.2, 127.7, 125.1, 124.7, 124.3, 122.7, 80.2, 80.1, 68.5, 68.4, 65.15, 65.09, 32.98, 32.97, 31.1, 30.9, 18.70, 18.68, 18.2.

IR (neat): 3390, 2960, 2928, 2873, 1090 cm⁻¹.

Anal. Calcd for C₁₃H₁₈O₂: C, 75.69; H, 8.80. Found: C, 75.31; H, 8.61.

### Example 32

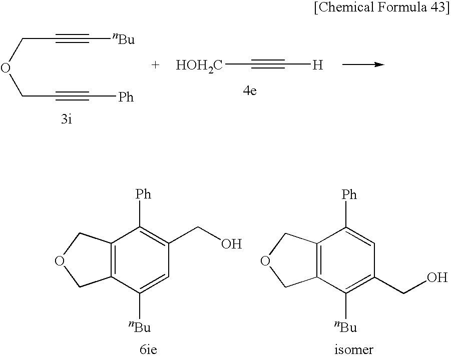

The substituted benzene 6ie was obtained from the compound 3i and compound 4e in a similar manner as in Example 4 (6ie:isomer=56:44, total yield: 96%).

6ie:

¹H-NMR (600 MHz, CDCl₃): δ 7.19-7.41 (m, 6H, Ph and Ar), 5.13 (s, 2H, ArCH₂O), 4.84 (s, 2H, ArCH₂O), 4.46 (s, 2H, ArCH₂OH), 2.54 (t, 2H, J=7.8 Hz, ArCH₂C), 1.60 (quint, 2H, J=7.8 Hz, CH₂CH₂CH₃), 1.39 (sext, 2H, J=7.8 Hz, CH₂CH₂CH₃), 0.95 (t, 3H, J=7.8 Hz, CH₂CH₂CH₃).

Isomer (Selected Peaks):

¹H-NMR (600 MHz, CDCl₃): δ 5.17 (s, 2H, ArCH₂O), 4.74 (s, 2H, ArCH₂OH), 2.58 (t, 2H, J=7.8 Hz, ArCH₂C), 1.50 (quint, 2H, J=7.8 Hz, CH₂CH₂CH₃), 1.41 (sext, 2H, J=7.8 Hz, CH₂CH₂CH₃).

¹³C-NMR (150 MHz, CDCl₃): δ 171.2, 139.9, 139.3, 138.4, 138.2, 138.1, 137.9, 136.8, 136.3, 135.5, 133.5, 133.4, 132.3, 128.6, 128.5, 128.4, 128.1, 127.6, 127.4, 127.2, 73.7, 73.6, 73.4, 73.1, 72.8, 62.5, 62.4, 60.4, 50.5, 33.0, 32.5, 32.2, 29.6, 23.0, 22.5, 20.9, 14.1, 13.9, 13.8. (measured on the mixture of 6ie and the isomer).

IR (neat): 3381, 2928, 2857, 1057 cm⁻¹. (measured on the mixture of 6ie and the isomer).

Anal. Calcd for C₁₉H₂₂O₂: C, 80.82; H, 7.85. Found: C, 80.61; H, 7.88. (measured on the mixture of 6ie and the isomer).

### Example 33

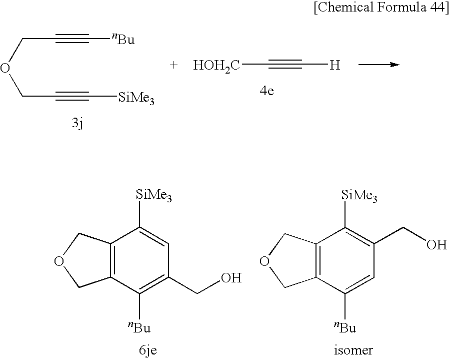

The substituted benzene 6je was obtained from the compound 3j and compound 4e in a similar manner as in Example 4 (6je:isomer=65:35, total yield: 94%).

6je:

¹H-NMR (600 MHz, CDCl₃): δ 7.40 (s, 1H, Ar), 5.14 (s, 2H, ArCH₂O), 5.10 (s, 2H, ArCH₂O), 4.73 (s, 2H, ArCH₂OH), 2.56-2.60 (m, 2H, ArCH₂CH₂), 1.50 (quint, 2H, J=7.8 Hz, CH₂CH₂CH₃), 1.41 (sext, 2H, J=7.8 Hz, CH₂CH₂CH₃), 0.94 (t, 3H, J=7.8 Hz, CH₂CH₂CH₃), 0.27 (s, 9H, SiMe₃).

Isomer:

¹H-NMR (600 MHz, CDCl₃): δ 7.26 (s, 1H, Ar), 5.14 (s, 2H, ArCH₂O), 5.05 (s, 2H, ArCH₂O), 4.75 (s, 2H, ArCH₂OH), 2.50 (t, 2H, J=7.8 Hz, ArCH₂CH₂), 1.57 (quint, 2H, J=7.8 Hz, CH₂CH₂CH₃), 1.37 (sext, 2H, J=7.8 Hz, CH₂CH₂CH₃), 0.93 (t, 3H, J=7.8 Hz, CH₂CH₂CH₃), 0.35 (s, 9H, SiMe₃).

¹³C-NMR (150 MHz, CDCl₃): δ 146.3, 144.1, 137.7, 137.3, 136.6, 136.4, 135.8, 134.0, 130.0, 127.9, 75.2, 74.3, 72.4, 71.9, 65.3, 62.8, 33.0, 32.5, 31.9, 24.2, 23.1, 22.6, 13.88, 13.86, 1.8, −1.0. (measured on the mixture of 6je and the isomer).

IR (neat): 3441, 2957, 2899, 1599, 1572, 1506, 1383, 1250, 1109, 1061 cm⁻¹. (measured on the mixture of 6je and the isomer).

Anal. Calcd for C₁₆H₂₆O₂Si: C, 69.01; H, 9.41. Found: C, 68.62; H, 9.68. (measured on the mixture of 6je and the isomer).

### Example 34

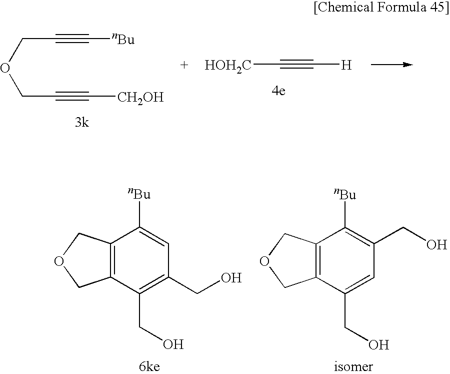

The substituted benzene 6ke was obtained from the compound 3k and compound 4e in a similar manner as in Example 4 (6ke:isomer=50:50, total yield: 85%).

6ke:

¹H-NMR (600 MHz, CDCl₃): δ 7.04 (s, 1H, Ar), 5.16 (s, 2H, ArCH₂O), 5.08 (s, 2H, ArCH₂O), 4.67 (s, 2H, ArCH₂OH), 4.55 (s, 2H, ArCH₂OH), 3.47-3.69 (br, 2H, OH), 2.48 (t, 2H, J=7.8 Hz, ArCH₂CH₂), 1.54 (quint, 2H, J=7.8 Hz, CH₂CH₂CH₃), 1.34 (sext, 2H, J=7.8 Hz, CH₂CH₂CH₃), 0.92 (t, 3H, J=7.8 Hz, CH₂CH₂CH₃).

¹³C-NMR (150 MHz, CDCl₃): δ 139.1, 139.0, 137.8, 136.2, 130.1, 129.4, 73.0, 72.9, 63.7, 60.1, 32.9, 32.1, 22.4, 12.8.

Isomer:

¹H-NMR (600 MHz, CDCl₃): δ 7.25 (s, 1H, Ar), 5.12 (s, 2H, ArCH₂O), 5.08 (s, 2H, ArCH₂O), 4.69 (s, 2H, ArCH₂OH), 4.54 (s, 2H, ArCH₂OH), 2.54 (t, 2H, J=7.8 Hz, ArCH₂CH₂), 2.39-2.50 (br, 1H, OH), 2.25-2.32 (br, 1H, OH), 1.45 (quint, 2H, J=7.8 Hz, CH₂CH₂CH₃), 1.39 (sext, 2H, J=7.8 Hz, CH₂CH₂CH₃), 0.93 (t, 3H, J=7.8 Hz, CH₂CH₂CH₃).

¹³C-NMR (150 MHz, CDCl₃): δ 139.1, 137.9, 136.8, 133.9, 131.7, 126.7, 72.9, 72.2, 63.1, 62.3, 32.5, 29.7, 23.0, 13.9.

IR (KBr): 3368, 3285, 2953, 2845, 1053 cm⁻¹. (measured on the mixture of 6ke and the isomer).

Anal. Calcd for C₁₄H₂O₃: C, 71.16; H, 8.53. Found: C, 70.85; H, 8.43. (measured on the mixture of 6ke and the isomer).

### Example 35

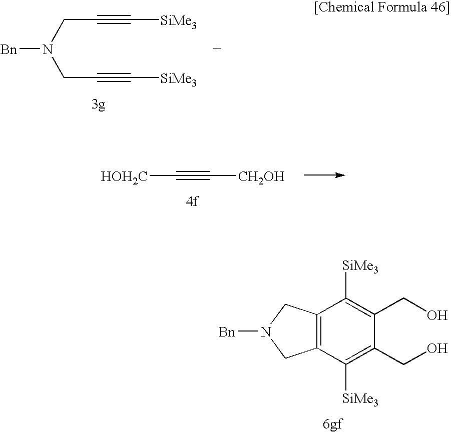

The substituted benzene 6gf was obtained from the compound 3g and compound 4f in a similar manner as in Example 4 (yield: 96%).

¹H-NMR (500 MHz, CDCl₃): δ 7.25-7.38 (m, 5H, Ph), 4.68 (s, 4H, ArCH₂N), 3.89 (s, 4H, ArCH₂OH), 3.86 (s, 2H, PhCH₂N), 0.33 (s, 18H, SiMe₃).

¹³C-NMR (150 MHz, CDCl₃): δ 145.3, 144.3, 138.3, 136.0, 128.8, 128.4, 127.2, 62.4, 60.4, 60.3, 2.5.

IR (KBr): 3337, 2949, 2897, 1252, 1022 cm⁻¹.

Mp: 55-57° C.

Anal. Calcd for C₂₃H₃₅NO₂Si₂: C, 66.77; H, 8.53; N, 3.39. Found: C, 66.49; H, 8.57; N, 3.14.

### Example 36

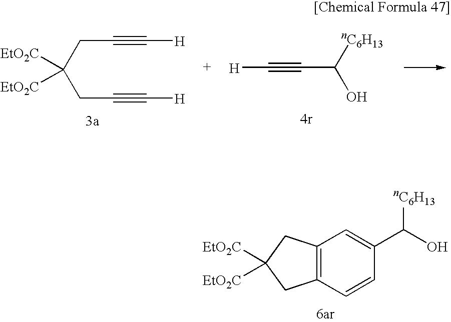

The substituted benzene 6ar was obtained from the compound 3a and compound 4r in a similar manner as in Example 4 (yield: 90%).

¹H-NMR (600 MHz, CDCl₃): δ 7.18 (s, 1H, Ar), 7.15 (d, 1H, J=7.5 Hz, Ar), 7.12 (d, 1H, J=7.5 Hz, Ar), 4.61 (t, 1H, J=6.6 Hz, ArCH₂OH), 4.20 (q, 4H, J=7.2 Hz, OCH₂CH₃), 3.57 (s, 4H, ArCH₂C), 1.82-1.86 (br, 1H, OH), 1.73-1.80 (m, 1H, alkyl), 1.63-1.70 (m, 1H, alkyl), 1.35-1.44 (m, 1H, alkyl), 1.21-1.34 (m, 7H, alkyl), 1.25 (t, 6H, J=7.2 Hz, OCH₂CH₃), 0.87 (t, 3H, J=7.2 Hz, CH₂CH₂CH₃).

¹³C-NMR (150 MHz, CDCl₃): δ 171.6, 143.9, 140.3, 139.3, 124.8, 124.0, 121.7, 74.6, 61.7, 60.4, 40.4, 40.2, 39.1, 31.7, 29.2, 25.8, 22.6, 14.02, 13.98.

IR (KBr): 3428, 2928, 2856, 1727, 1250, 1155 cm⁻¹.

Anal. Calcd for C₂₂H₃₂O₅: C, 70.18; H, 8.57. Found: C, 70.33; H, 8.26.

### Example 37

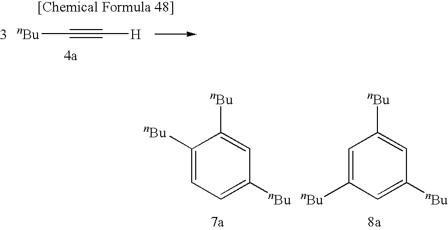

Zinc powder (13.0 mg, 0.20 mmol) and the compound 4a (164 mg, 2.0 mmol) were dissolved in THF (5 mL), followed by the addition of a solution of CoCl₂-6H₂O (23.8 mg, 0.10 mmol) and dipimp (32 mg, 0.12 mmol) in THF (3 mL). The resulting mixed solution was warmed for 5 minutes at 35 to 40° C., and was then stirred at room temperature for 4 hours. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzenes 7a and 8a (7a:8a=63:37, total yield: 91%).

7a (Selected Peaks):

¹H-NMR (600 MHz, CDCl₃): δ 7.04 (d, 1H, J=7.2 Hz, Ar), 6.95 (s, 1H, Ar), 6.93 (d, 1H, J=7.2 Hz, Ar).

¹³C-NMR (150 MHz, CDCl₃): δ 140.3, 140.1, 137.6, 129.2, 128.9, 125.7, 35.3, 33.78, 33.75, 33.59, 33.57, 32.5, 32.0, 22.89, 22.86, 22.53, 22.49, 14.0.

8a (Selected Peaks):

¹H-NMR (600 MHz, CDCl₃): δ 6.81 (s, 3H, Ar), 2.53-2.59 (m, 6H, ArCH₂C), 1.51-1.62 (m, 6H, CH₂CH₂CH₃), 1.30-1.43 (m, 6H, CH₂CH₂CH₃), 0.90-0.98 (m, 6H, CH₂CH₂CH₃).

¹³C-NMR (150 MHz, CDCl₃): δ 142.7, 125.8, 35.7, 33.8, 22.5, 14.0.

IR (neat): 2950, 2935, 1603, 1501, 1454 cm⁻¹. (measured on the mixture of 7a and 8a).

Anal. Calcd. for C₁₈H₃₀: C, 87.73; H, 12.27. Found: C, 87.37; H, 12.15. (measured on the mixture of 7a and 8a).

### Example 38

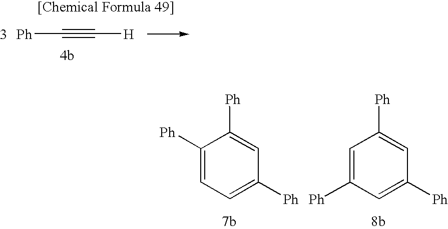

The substituted benzenes 7b were obtained from the compound 4b in a similar manner as in Example 37 (7b:8b>99:1, yield: 91%).

7b:

IR (KBr): 3080, 3057, 3032, 1633, 1628, 1593, 1498 cm⁻¹.

Mp: 185-188° C. (reference value 172-174° C.).

Anal. Calcd for C₂₄H₁₈: C, 94.08; H, 5.92. Found: C, 94.12; H, 5.63.

### Example 39

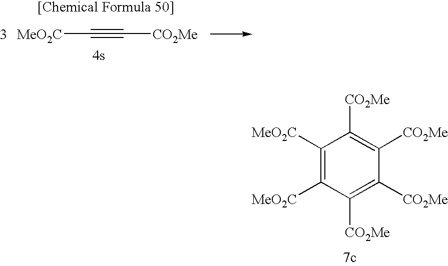

The substituted benzene 7c was obtained from the compound 4s in a similar manner as in Example 37 (yield: 98%).

¹H-NMR (500 MHz, CDCl₃): δ 3.89 (s, 18H, Me).

¹³C-NMR (125 MHz, CDCl₃): δ 165.0, 133.8, 53.4.

IR (neat): 2959, 1728, 1231 cm⁻¹.

Mp: 203-204° C.

Anal. Calcd for C₁₈H₁₈O₁₂: C, 50.71; H, 4.26. Found: C, 51.01; H, 4.31.

### Example 40

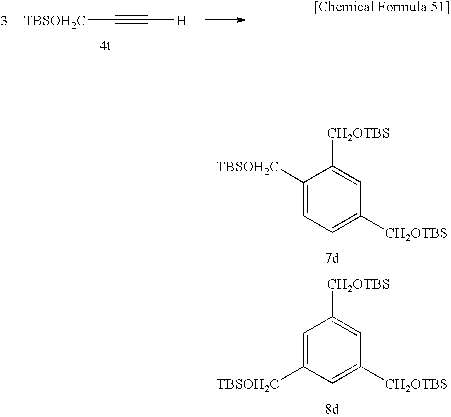

The substituted benzenes 7d and 8d were obtained from the compound 4t in a similar manner as in Example 37 (7d:8d=60:40, total yield: 88%).

8d:

¹H-NMR (600 MHz, CDCl₃): δ 7.19 (s, 3H, Ar), 4.76 (s, 6H, ArCH₂O), 0.94 (s, 27H, t-Bu), 0.10 (s, 18H, Me).

¹³C-NMR (150 MHz, CDCl₃): δ 141.3, 122.4, 65.0, 26.0, 18.4, −5.2.

7d:

¹H-NMR (600 MHz, CDCl₃): δ 7.43 (s, 1H, Ar), 7.39 (d, 1H, J=7.2 Hz, Ar), 7.23 (d, 1H, J=7.2 Hz, Ar), 4.78 (s, 2H, ArCH₂O), 4.76 (s, 4H, ArCH₂O), 0.94 (s, 27H, t-Bu), 0.10 (s, 18H, Me).

¹³C-NMR (150 MHz, CDCl₃): δ 140.1, 138.2, 136.7, 126.6, 124.6, 124.4, 65.1, 62.83, 62.81, 26.0, 18.4, −5.2.

IR (neat): 2950, 2935, 2850, 1362, 1255, 1087 cm⁻¹. (measured on the mixture of 7d and 8d).

Anal. Calcd for C₂₇H₅₄O₃Si₃: C, 63.47; H, 10.65. Found: C, 63.84; H, 10.61. (measured on the mixture of 7d and 8d).

### Example 41

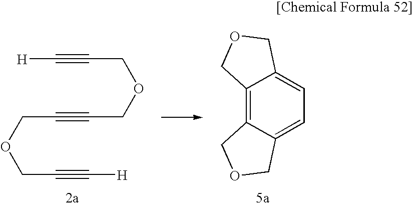

Zinc powder (6.5 mg, 0.10 mmol) and the compound 2a (1.0 mmol) were dissolved in THF (2.5 mL), followed by the addition of a solution of FeCl₃-6H₂O (13.5 mg, 0.05 mmol) and dipimp (16.0 mg, 0.06 mmol) in THF (1.5 mL). The resulting mixed solution was stirred at 50° C. for 24 hours. After completion of the reaction, the reaction mixture was allowed to cool down to room temperature. Diethyl ether (10 mL) was added, and the resulting mixture was filtered through “Celite”. The filtrate was concentrated under reduced pressure, and the concentrate was purified by chromatography on a silica gel column to obtain the substituted benzene 5a (yield: 95%).

### Example 42

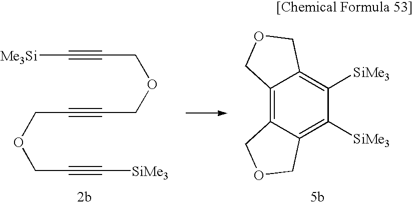

The substituted benzene 5b was obtained from the compound 2b in a similar manner as in Example 41 except for stirring at 50° C. for 48 hours (yield: 24%).

### Example 43

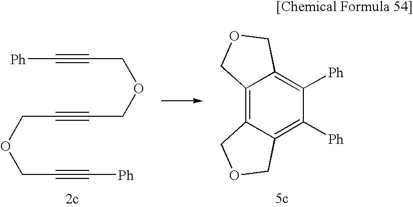

The substituted benzene 5c was obtained from the compound 2c in a similar manner as in Example 41 except for stirring at 50° C. for 48 hours (yield: 82%).

### Example 44

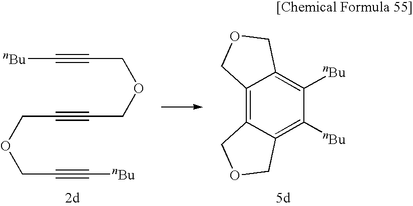

The substituted benzene 5d was obtained from the compound 2d in a similar manner as in Example 41 except for stirring at 50° C. for 48 hours (yield: 64%).

¹H-NMR (500 MHz, CDCl₃): δ 5.11 (s, 4H, ArCH₂O), 5.02 (s, 4H, ArCH₂O), 2.49-2.53 (m, 4H, ArCH₂CH₂), 1.37-1.50 (m, 8H, CH₂CH₂CH₃), 0.95 (t, 6H, J=7.0 Hz, CH₂CH₂CH₃).

¹³C-NMR (125 MHz, CDCl₃): δ 138.3, 133.3, 129.3, 73.0, 72.7, 32.7, 29.6, 23.0, 13.9.

IR (neat): 2956, 2930, 2866, 1461, 1056 cm⁻¹.

Mp: 93-95° C.

Anal. Calcd for C₁₈H₂₆O₂: C, 78.79; H, 9.55. Found: C, 78.54; H, 9.70.

### Example 45

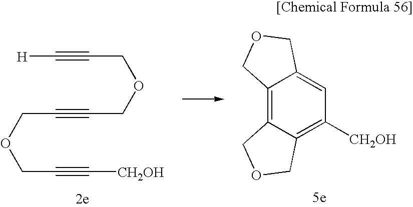

The substituted benzene 5e was obtained from the compound 2e in a similar manner as in Example 41 except for stirring at 50° C. for 48 hours (yield: 82%).

¹H-NMR (600 MHz, CDCl₃): δ 7.14 (s, 1H, Ar), 5.16 (s, 2H, ArCH₂O), 5.10 (s, 2H, ArCH₂O), 5.01 (brs, 4H, ArCH₂O), 4.65 (d, 2H, J=5.4 Hz, ArCH₂OH), 2.06 (t, 1H, J=5.4 Hz, OH).

¹³C-NMR (150 MHz, CDCl₃): δ 139.3, 137.0, 133.7, 132.6, 131.5, 118.4, 73.3, 72.5, 72.14, 72.08, 63.3.

IR (KBr): 3400, 2855, 1640, 1618, 1086, 1040 cm⁻¹.

Mp: 135-137° C.

Anal. Calcd for C₁₁H₁₂O₃: C, 68.74; H, 6.29. Found: C, 68.49, H, 6.41.

### Example 46

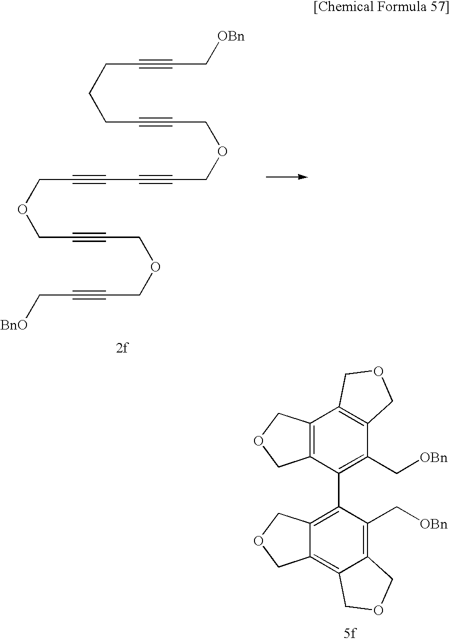

The substituted benzene 5f was obtained from the compound 2f in a similar manner as in Example 41 except for stirring at 50° C. for 48 hours (yield: 81%).

¹H-NMR (500 MHz, CDCl₃): δ 7.24-7.29 (m, 6H, Ph), 7.13-7.16 (m, 4H, Ph), 5.20 (d, 2H, J=14.0 Hz, ArCH₂O), 5.17 (d, 2H, J=14.0 Hz, ArCH₂O), 5.03-5.11 (m, 8H, ArCH₂O), 4.72 (d, 2H, J=12.0 Hz, ArCH₂O), 4.64 (d, 2H, J=12.0 Hz, ArCH₂O), 4.32 (d, 2H, J=11.5 Hz, ArCH₂OBn), 4.29 (d, 2H, J=11.5 Hz, ArCH₂OBn), 4.12 (d, 2H, J=11.0 Hz, PhCH₂O), 4.08 (d, 2H, J=11.0 Hz, PhCH₂O).

¹³C-NMR (125 MHz, CDCl₃): δ 139.7, 138.2, 137.5, 132.5, 131.9, 130.0, 129.0, 128.3, 127.74, 127.71, 73.3, 73.2, 73.1, 72.8, 72.2, 67.6.

IR (KBr): 3057, 3032, 2889, 2855, 1775, 1767, 1458, 1450, 1350, 1087, 1048 cm⁻¹.

Mp: 150-159° C.

Anal. Calcd for C₃₆H₃₄O₆: C, 76.85; H, 6.09. Found: C, 76.87, H, 6.06.

### Example 47

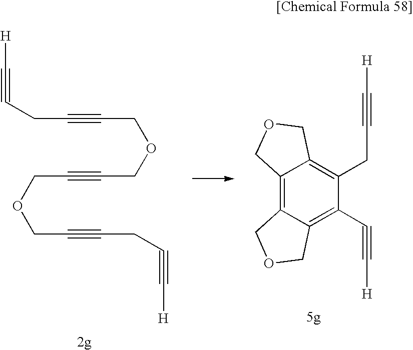

The substituted benzene 5g was obtained from the compound 2g in a similar manner as in Example 41 except for stirring at 50° C. for 48 hours (yield: 77%).

¹H-NMR (500 MHz, CDCl₃): δ 5.20 (s, 4H, ArCH₂O), 5.04 (s, 4H, ArCH₂O), 3.55 (d, 4H, J=2.9 Hz, ArCH₂C≡C), 2.08 (t, 2H, J=2.9 Hz, C≡CH).

¹³C-NMR (125 MHz, CDCl₃): δ 140.1, 132.4, 128.3, 81.0, 73.8, 73.6, 71.0, 20.2.

IR (KBr): 3244, 2854, 1060, 1041 cm⁻¹.

### Example 48

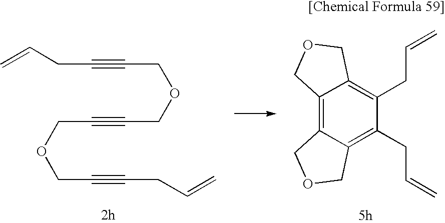

The substituted benzene 5h was obtained from the compound 2h in a similar manner as in Example 41 except for stirring at 50° C. for 48 hours (yield: 93%).

¹H-NMR (600 MHz, CDCl₃): δ 5.81-5.89 (m, 2H, CH₂CH₂═CH₂), 5.08 (s, 4H, ArCH₂O), 5.01-5.05 (m, 6H, ArCH₂O and CH═CH₂), 4.91 (d, 2H, J=9.6 Hz, CH═CH₂), 3.30 (d, 4H, J=6.0 Hz, ArCH₂CH).

¹³C-NMR (150 MHz, CDCl₃): δ 139.1, 135.1, 130.3, 130.1, 115.7, 72.9, 72.7, 33.9.

IR (KBr): 3071, 2847, 1636, 1055 cm⁻¹.

Mp: 53-55° C.

Anal. Calcd for C₁₆H₁₈O₂: C, 79.31; H, 7.49. Found: C, 79.29; H, 7.20.

### Example 49

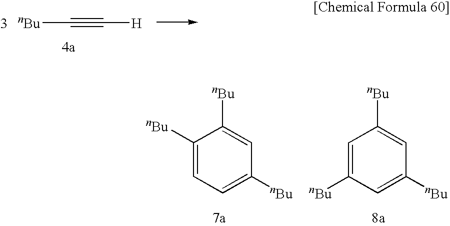

Zinc powder (13.0 mg, 0.20 mmol) and the compound 4a (164 mg, 2.0 mmol) were dissolved in THF (5 mL), followed by the addition of a solution of NiCl₂-6H₂O (23.8 mg, 0.10 mmol) and dipimp (32 mg, 0.12 mmol) in THF (3 mL). The resulting mixed solution was stirred at 40° C. for 20 hours. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzenes 7a and 8a (7a:8a=24:76, total yield: 94%).

### Example 50

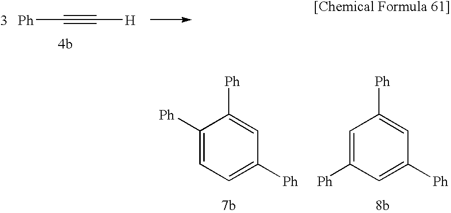

The substituted benzenes 7b and 8b were obtained from the compound 4b in a similar manner as in Example 49 (7b:8b=65:35, total yield: 88%).

### Example 51

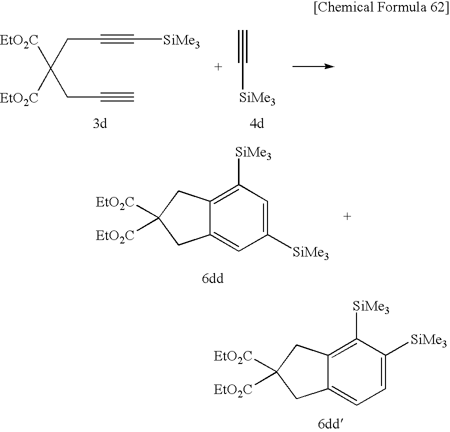

The substituted benzenes 6dd and 6dd′ were obtained from the compound 3d and compound 4d in a similar manner as in Example 4 except for stirring at room temperature for 12 hours (yield: 93% (58:42)).

¹H-NMR (500 MHz, CDCl₃):

6dd: δ 7.43 (d, 1H, J=7.5 Hz, Ar), 7.10 (d, 1H, J=7.5 Hz, Ar), 4.16-4.10 (m, 4H, OCH₂CH₃), 3.61 (s, 2H, ArCH₂C), 3.45 (s, 2H, ArCH₂C), 1.21-1.16 (m, 6H, OCH₂CH₃), 0.36 (s, 9H, Si(CH₃)₃), 0.28 (s, 9H, Si(CH₃)₃).

6dd′: δ 7.39 (s, 1H, Ar), 7.30 (s, 1H, Ar), 4.16-4.10 (m, 4H, OCH₂CH₃), 3.56 (s, 2H, ArCH₂C), 3.51 (s, 2H, ArCH₂C), 1.21-1.16 (m, 6H, OCH₂CH₃), 0.25 (s, 9H, Si(CH₃)₃), 0.18 (s, 9H, Si(CH₃)₃)

Thin layer chromatography (TLC) (Merk 5554): Rf=0.73 (hexane:Et₂O=1:1 (v/v)).

### Example 52

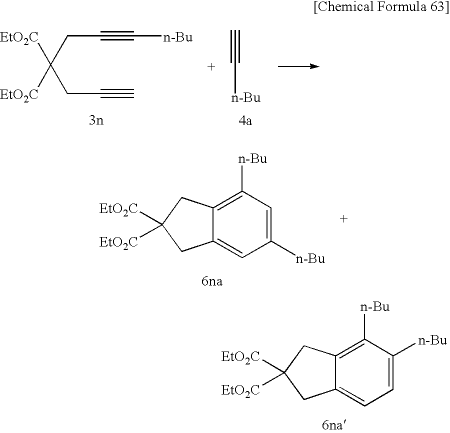

The substituted benzenes 6na and 6na′ were obtained from the compound 3n and compound 4a in a similar manner as in Example 4 except for stirring at room temperature for 12 hours (yield: 64% (17:83)).

¹H-NMR (500 MHz, CDCl₃):

6na: δ 6.98-6.49 (m, 2H, Ar), 4.10 (q, 4H, J=7.0 Hz, OCH₂CH₃), 3.56 (s, 2H, ArCH₂C), 3.54 (s, 2H, ArCH₂C), 2.58-2.50 (m, 4H, Alkyl), 1.59-1.30 (m, 8H, Alkyl), 1.26 (t, 6H, J=7.0 Hz, OCH₂CH₃), 0.98-0.88 (m, 6H, Alkyl).

6na′: δ 6.85 (s, 1H, Ar), 6.79 (s, 1H, Ar), 4.10 (q, 4H, J=7.0 Hz, OCH₂CH₃), 3.56 (s, 2H, ArCH₂C), 3.50 (s, 2H, ArCH₂C), 2.58-2.50 (m, 4H, Alkyl), 1.59-1.30 (m, 8H, Alkyl), 1.26 (t, 6H, J=7.0 Hz, OCH₂CH₃), 0.98-0.88 (m, 6H, Alkyl).

Thin layer chromatography (TLC) (Merk 5554): Rf=0.78 (hexane:Et₂O=1:1 (v/v)).

### Example 53

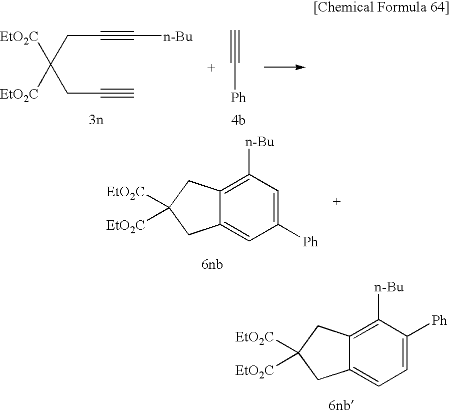

The substituted benzenes 6nb and 6nb′ were obtained from the compound 3n and compound 4b in a similar manner as in Example 4 except for stirring at room temperature for 8 hours (yield: 95% (70:30)).

¹H-NMR (500 MHz, CDCl₃):

6nb: δ 7.59-7.02 (m, 7H, Ar), 4.28-4.22 (m, 4H, OCH₂CH₃), 3.65 (s, 2H, ArCH₂C), 3.63 (s, 2H, ArCH₂C) 2.54-2.50 (m, 2H, Alkyl), 1.46-1.34 (m, 2H, Alkyl), 1.29 (t, 6H, J=7.0 Hz, OCH₂CH₃), 1.25-1.16 (m, 2H, Alkyl), 0.78 (t, 3H, J=7.2 Hz, Alkyl).

6nb′: δ 7.59-7.02 (m, 7H, Ar), 4.28-4.22 (m, 4H, OCH₂CH₃), 3.66 (s, 2H, ArCH₂C), 3.59 (s, 2H, ArCH₂C), 2.64 (t, 2H, J=7.5 Hz, Alkyl), 1.66-1.58 (m, 2H, Alkyl), 1.25-1.16 (m, 2H, Alkyl), 1.29 (t, 6H, J=7.0 Hz, OCH₂CH₃), 0.96 (t, 3H, J=7.2 Hz, Alkyl).

Thin layer chromatography (TLC) (Merk 5554): Rf=0.52 (hexane:Et₂O=1:1 (v/v)).

### Example 54

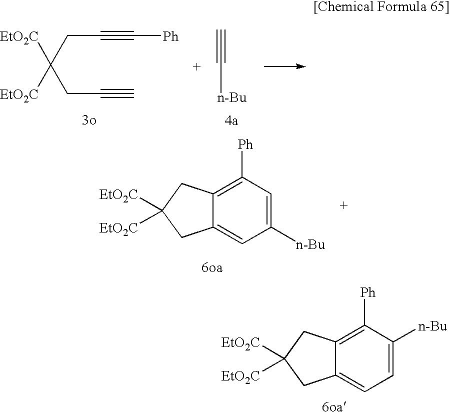

The substituted benzenes 6oa and 6oa′ were obtained from the compound 3o and compound 4a in a similar manner as in Example 4 except for stirring at room temperature for 8 hours (yield: 58% (30:70)).

¹H-NMR (500 MHz, CDCl₃):

6oa: δ 7.45-6.99 (m, 7H, Ar), 4.21-4.12 (m, 4H, OCH₂CH₃), 3.61 (s, 2H, ArCH₂C), 3.25 (s, 2H, ArCH₂C), 2.41-2.35 (m, 2H, Alkyl), 1.42-1.32 (m, 2H, Alkyl), 1.21 (t, 6H, J=7.2 Hz, OCH₂CH₃), 1.20-1.12 (m, 2H, Alkyl), 0.74 (t, 3H, J=7.5 Hz, Alkyl).

6oa′: δ 7.45-6.99 (m, 7H, Ar), 4.21-4.12 (m, 4H, OCH₂CH₃), 3.61 (s, 2H, ArCH₂C), 3.60 (s, 2H, ArCH₂C), 2.60 (t, 2H, J=7.5 Hz, Alkyl), 1.64-1.52 (m, 2H, Alkyl), 1.42-1.32 (m, 2H, Alkyl), 1.22 (t, 6H, J=7.2 Hz, OCH₂CH₃), 0.92 (t, 3H, J=7.5 Hz, Alkyl).

Thin layer chromatography (TLC) (Merk 5554): Rf=0.66 (hexane:Et₂O=1:1 (v/v)).

### Example 55

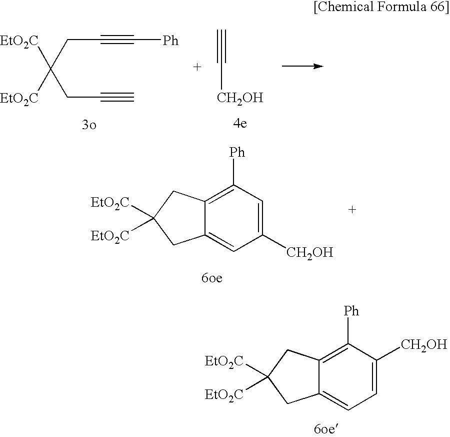

The substituted benzenes 6oe and 6oe′ were obtained from the compound 3o and compound 4e in a similar manner as in Example 4 except for stirring at room temperature for 8 hours (yield: 66% (33:64)).

¹H-NMR (500 MHz, CDCl₃) :

6oe: δ 7.45-7.18 (m, 7H, Ar), 4.44 (d, 2H, J=4.5 Hz, CH₂OH), 4.20-4.14 (m, 4H, OCH₂CH₃), 3.64 (s, 2H, ArCH₂C), 3.30 (s, 2H, ArCH₂C), 1.48-1.41 (br, 1H, CH₂OH), 1.28-1.19 (m, 6H, OCH₂CH₃).

6oe′: δ 7.45-7.18 (m, 7H, Ar), 4.69 (d, 2H, J=4.5 Hz, CH₂OH), 4.20-4.14 (m, 4H, OCH₂CH₃), 3.64 (s, 2H, ArCH₂C), 3.62 (s, 2H, ArCH₂C), 1.74-1.66 (br, 1H, CH₂OH), 1.28-1.19 (m, 6H, OCH₂CH₃).

Thin layer chromatography (TLC) (Merk 5554): Rf=0.14 (hexane:Et₂O=1:1 (v/v)).

### Example 56

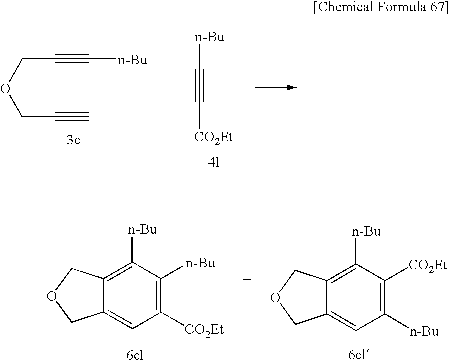

The substituted benzenes 6cl and 6cl′ were obtained from the compound 3c and compound 4l in a similar manner as in Example 4 except for stirring at room temperature for 8 hours (yield: 39% (84:16)).

¹H-NMR (500 MHz, CDCl₃):

6cl: δ 6.94 (s, 1H, Ar), 5.08 (s, 4H, ArCH₂O), 4.38 (q, 2H, J=7.0 Hz, OCH₂CH₃), 2.61-2.44 (m, 4H, Alkyl), 1.60-1.26 (m, 8H, Alkyl), 0.96-0.87 (m, 6H, Alkyl).

6cl′: δ 7.46 (s, 1H, Ar), 5.10 (s, 4H, ArCH₂O), 4.21 (q, 2H, J=7.0 Hz, OCH₂CH₃), 2.61-2.44 (m, 4H, Alkyl), 1.60-1.26 (m, 8H, Alkyl), 0.96-0.87 (m, 6H, Alkyl).

Thin layer chromatography (TLC) (Merk 5554): Rf=0.70 (hexane:Et₂O=1:1 (v/v)).

### Example 57

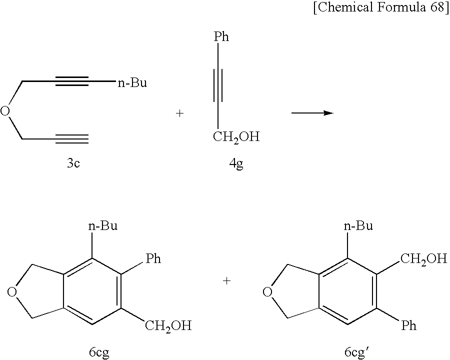

The substituted benzenes 6cg and 6cg′ were obtained from the compound 3c and compound 4g in a similar manner as in Example 4 except for stirring at room temperature for 8 hours (yield: 6%).

¹H-NMR (500 MHz, CDCl₃): δ 7.42 (t, 2H, J=7.2 Hz, Ph), 7.37 (t, 1H, J=7.2 Hz, Ph), 7.26 (s, 1H, Ar), 7.18 (d, 2H, J=7.2 Hz, Ph), 5.18 (s, 2H, ArCH₂O), 5.15 (s, 2H, ArCH₂O), 4.34 (s, 2H, ArCH₂OH), 2.26-2.21 (m, 2H, Alkyl), 1.64-1.52 (br, 1H, CH₂OH), 1.30-1.23 (m, 2H, Alkyl), 1.16-1.09 (m, 2H, Alkyl), 0.71 (t, 3H, J=7.5 Hz, Alkyl).

Thin layer chromatography (TLC) (Merk 5554): Rf=0.13 (hexane:Et₂O=1:1 (v/v)).

### Example 58

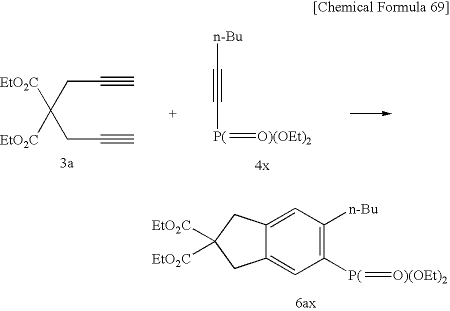

The substituted benzene 6ax was obtained from the compound 3a and compound 4x in a similar manner as in Example 4 except for stirring at room temperature for 12 hours (yield: 66%).

¹H-NMR (600 MHz, CDCl₃): δ 7.71 (d, 2H, J=7.8 Hz, Ar), 7.09 (d, 1H, J=6.5 Hz, Ar), 4.16 (q, 4H, J=6.8 Hz, COCH₂CH₃), 4.08-4.12 (m, 2H, POCH₂CH₃), 4.01-4.06 (m, 2H, POCH₂CH₃), 3.53 (s, 2H, ArCH₂C), 3.52 (s, 2H, ArCH₂C), 2.81 (t, 2H, J=7.8 Hz, ArCH₂C), 1.56-1.52 (m, 2H, CH₂CH₂CH₃), 1.39-1.34 (m, 2H, CH₂CH₂CH₃), 1.27 (t, 6H, J=7.0 Hz, POCH₂CH₃), 1.20 (t, 6H, J=7.2 Hz, COCH₂CH₃), 0.89 (t, 3H, J=7.1 Hz, CH₂CH₂CH₃).

¹³C-NMR (68 MHz, CDCl₃): δ 171.2, 145.9 (d, J_{P-C}=11.3 Hz), 145.0, 137.1 (d, J_{P-C}=16.2 Hz), 129.6 (d, J_{P-C}=10.6 Hz), 125.9 (d, J_{P-C}=14.5 Hz), 123.5, 61.82, 60.36, 40.66, 40.09, 34.12, 33.93, 23.04, 16.46, 14.10.

³¹P-NMR (242 MHz, CDCl₃): δ 21.06.

### Example 59

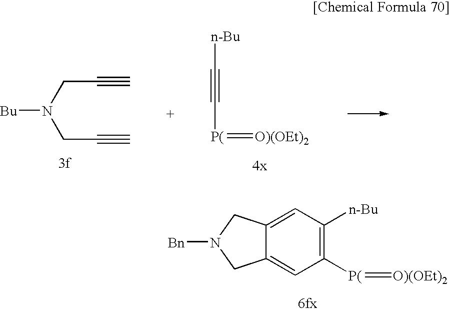

The substituted benzene 6fx was obtained from the compound 3f and compound 4x in a similar manner as in Example 4 except for stirring at room temperature for 12 hours (yield: 62%).

¹H-NMR (600 MHz, CDCl₃): δ 7.75 (d, 1H, J=8.1 Hz, Ar), 7.39 (d, 1H, J=7.9 Hz, Ph), 7.34 (t, 2H, J=7.8 Hz, Ph), 7.27 (d, 2H, J=7.8 Hz, Ph), 7.12 (d, 1H, J=7.8 Hz, Ar), 4.08-4.14 (m, 2H, OCH₂CH₃), 3.98-4.07 (m, 2H, OCH₂CH₃), 3.92 (s, 2H, ArCH₂N), 3.91 (s, 2H, PhCH₂N), 3.90 (s, 2H, ArCH₂N), 2.88 (t, 2H, J=7.6 Hz, ArCH₂C), 1.54-1.58 (m, 2H, tt, CH₂CH₂CH₃), 1.40-1.42 (m, 2H, CH₂CH₂CH₃), 1.30 (t, 6H, J=7.0 Hz, OCH₂CH₃), 0.94 (t, 3H, J=7.2 Hz, CH₂CH₂CH₃).

¹³C-NMR (68 MHz, CDCl₃): δ 145.8 (d, J_{P-C}=12.3 Hz), 145.1, 138.8, 137.2 (d, J_{P-C}=15.1 Hz), 128.6 (d, J_{P-C}=21.2 Hz), 127.5 (d, J_{P-C}=12.8 Hz), 127.1, 126.9, 124.3 (d, J_{P-C}=16.8 Hz), 123.9, 62.10, 61.86, 60.24, 58.89, 58.50, 34.24 (d, J_{P-C}=13.5 Hz), 23.01, 16.48, 14.11.

³¹P-NMR (242 MHz, CDCl₃): δ 21.06.

### Example 60

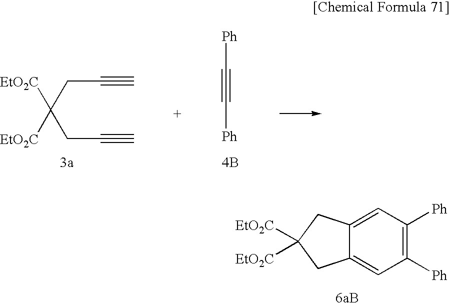

The substituted benzene 6aB was obtained from the compound 3a and compound 4B in a similar manner as in Example 4 except for stirring at room temperature for 12 hours (yield: 52%).

¹H-NMR (500 MHz, CDCl₃): δ 7.18-6.94 (m, 12H, Ar), 4.14 (q, 4H, J=7.0 Hz, OCH₂CH₃), 3.59 (s, 4H, ArCH₂C), 1.18 (t, 6H, J=7.0 Hz, OCH₂CH₃).

Thin layer chromatography (TLC) (Merk 5554): Rf=0.56 (hexane:Et₂O=1:1 (v/v)).

### Example 61

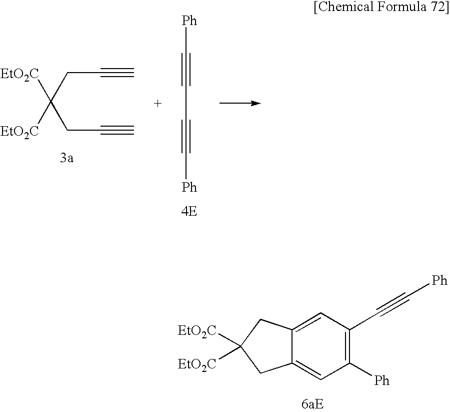

The substituted benzene 6aE was obtained from the compound 3a and compound 4E in a similar manner as in Example 4 except for stirring at room temperature for 8 hours (yield: 92%).

¹H-NMR (500 MHz, CDCl₃): δ 7.61 (d, 2H, J=7.5 Hz, Ar), 7.47 (s, 1H, Ar), 7.42 (t, 2H, J=7.5 Hz, Ar), 7.36 (t, 1H, J=7.5 Hz, Ar), 7.30-7.24 (m, 6H, Ar), 4.22 (q, 4H, J=7.0 Hz, OCH₂CH₃), 3.638 (s, 2H, ArCH₂C), 3.630 (s, 2H, ArCH₂C), 1.26 (t, 6H, J=7.0 Hz, OCH₂CH₃).

¹³C-NMR (125 MHz, CDCl₃): δ 171.3, 143.0, 140.9, 140.5, 139.0, 131.2, 129.3, 128.2, 128.1, 127.8, 127.7, 127.2, 125.2, 123.5, 120.2, 91.5, 89.6, 61.7, 60.4, 40.4, 39.9, 13.9.

Thin layer chromatography (TLC) (Merk 5554): Rf=0.52 (hexane:Et₂O=1:1 (v/v)).

### Example 62

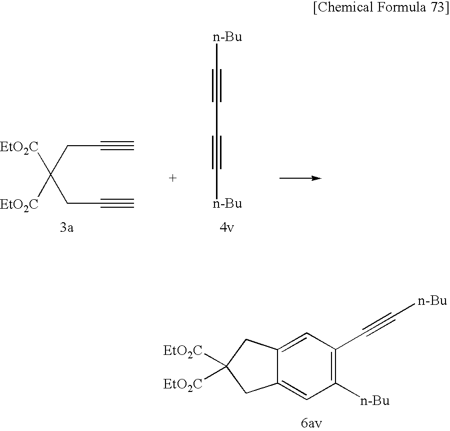

The substituted benzene 6av was obtained from the compound 3a and compound 4v in a similar manner as in Example 4 except for stirring at room temperature for 8 hours (yield: 99%).

¹H-NMR (500 MHz, CDCl₃): δ 7.18 (s, 1H, Ar), 6.98 (s, 1H, Ar), 4.18 (q, 4H, J=7.2 Hz, OCH₂CH₃), 3.52 (s, 2H, ArCH₂C), 3.50 (s, 2H, ArCH₂C), 2.71-2.67 (m, 2H, Alkyl), 2.42 (t, 2H, J=7.0 Hz, Alkyl), 1.61-1.33 (m, 8H, Alkyl), 1.24 (t, 6H, J=7.0 Hz, OCH₂CH₃), 0.94 (t, 3H, J=7.0 Hz, Alkyl), 0.93 (t, 3H, J=7.0 Hz, Alkyl).

¹³C-NMR (125 MHz, CDCl₃): δ 171.5, 143.6, 139.6, 137.1, 127.5, 124.3, 121.9, 92.9, 79.4, 61.6, 60.4, 40.3, 39.9, 34.2, 32.9, 30.9, 22.6, 21.9, 19.1, 13.98, 13.94, 13.5.

Thin layer chromatography (TLC) (Merk 5554): Rf=0.55 (hexane:Et₂O=1:1 (v/v)).

### Example 63

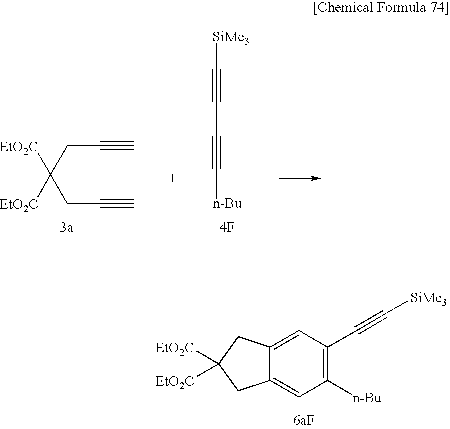

The substituted benzene 6aF was obtained from the compound 3a and compound 4F in a similar manner as in Example 4 (yield: 65%).

¹H-NMR (500 MHz, CDCl₃): δ 7.18 (s, 1H, Ar), 6.93 (s, 1H, Ar), 4.11 (q, 4H, J=7.2 Hz, OCH₂CH₃), 3.46 (s, 2H, ArCH₂C), 3.43 (s, 2H, ArCH₂C), 2.64 (t, 2H, J=7.8 Hz, Alkyl), 1.54-1.48 (m, 2H, Alkyl), 1.35-1.26 (m, 2H, Alkyl), 1.17 (t, 6H, J=7.2 Hz, OCH₂CH₃), 1.59 (s, 9H, Si(CH₃)₃).

¹³C-NMR (125 MHz, CDCl₃): δ 171.4, 144.5, 140.9, 137.2, 127.8, 124.5, 121.0, 104.3, 96.8, 61.6, 60.4, 40.4, 39.8, 34.3, 32.9, 22.6, 13.9, 13.8, −0.05.

Thin layer chromatography (TLC) (Merk 5554): Rf=0.64 (hexane:Et₂O=1:1 (v/v)).

### Example 64

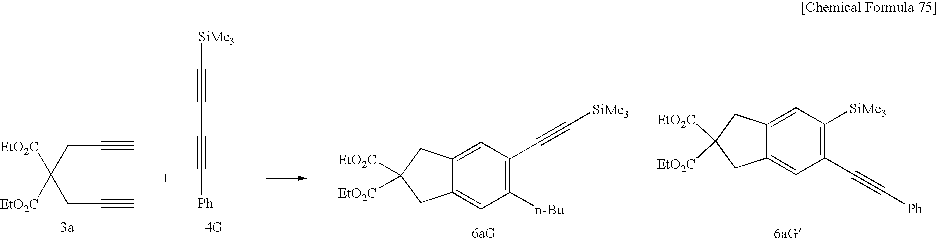

The substituted benzenes 6aG and 6aG′ were obtained from the compound 3a and compound 4G in a similar manner as in Example 4 except for stirring at room temperature for 8 hours (yield: 65% (80:20)).

¹H-NMR (500 MHz, CDCl₃):

6aG: δ 7.62-7.17 (m, 7H, Ar), 4.21 (q, 4H, J=7.0 Hz, OCH₂CH₃), 3.60 (s, 4H, ArCH₂C), 1.26 (t, 6H, J=7.0 Hz, OCH₂CH₃), 0.10 (s, 9H, Si(CH₃)₃).

6aG′: δ 7.63-7.21 (m, 7H, Ar), 4.22 (q, 4H, J=7.0 Hz, OCH₂CH₃), 3.63 (s, 4H, ArCH₂C), 1.27 (t, 6H, J=7.0 Hz, OCH₂CH₃), 0.07 (s, 9H, Si(CH₃)₃).

¹³C-NMR (125 MHz, CDCl₃):

6aG: δ 171.4, 143.4, 141.2, 140.3, 138.9, 129.3, 128.7, 127.6, 127.2, 125.2, 105.0, 96.8, 61.8, 60.4, 40.4, 39.9, 14.0, −0.3.

6aG′: δ 171.4, 143.1, 141.0, 131.2, 129.4, 128.3, 128.2, 128.0, 127.8, 127.3, 125.3, 91.6, 89.7, 61.8, 60.5, 40.5, 40.0, 14.0, 1.0.

Thin layer chromatography (TLC) (Merk 5554):

6aG: Rf=0.51 (hexane:Et₂O=1:1 (v/v)),

6aG′: Rf=0.49 (hexane:Et₂O=1:1 (v/v)).

### Example 65

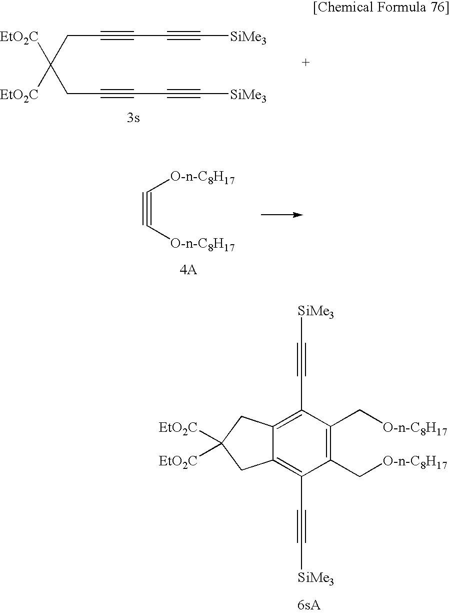

The substituted benzene 6sA was obtained from the compound 3s and compound 4A in a similar manner as in Example 4 (yield: 86%).

¹H-NMR (500 MHz, CDCl₃): δ 4.75 (s, 4H, ArCH₂O), 4.23 (q, 4H, J=7.2 Hz, OCH₂CH₃), 3.67 (s, 4H, ArCH₂C), 3.48 (t, 4H, J=7.2, OCH₂CH₂), 1.62-1.54 (m, 4H, Alkyl), 1.37-1.22 (m, 26H, Alkyl and OCH₂CH₃), 1.13 (s, 21H, Si(CH(CH₃)₂)₃ and Si(CH(CH₃)₂)₃), 0.89-0.85 (m, 6H, Alkyl), 0.39 (s, 18H, Si(CH₃)₃).

Thin layer chromatography (TLC) (Merk 5554): Rf=0.79 (hexane:Et₂O=1:1 (v/v)).

### Example 66

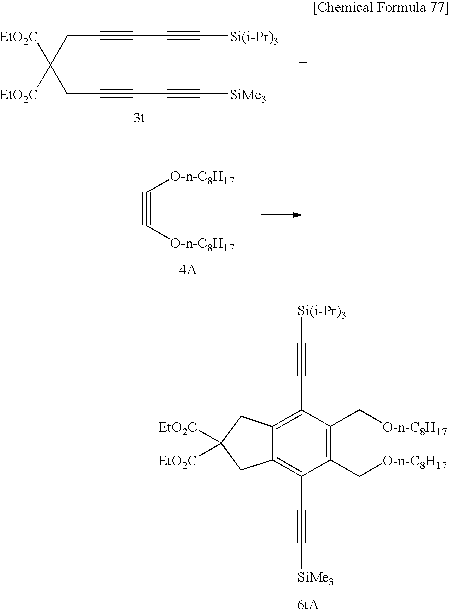

The substituted benzene 6tA was obtained from the compound 3t and compound 4A in a similar manner as in Example 4 except for stirring at room temperature for 8 hours (yield: 66%).

¹H-NMR (500 MHz, CDCl₃): δ 4.77 (s, 2H, ArCH₂O), 4.53 (s, 2H, ArCH₂O), 4.22-4.15 (m, 4H, OCH₂CH₃), 3.64 (s, 2H, ArCH₂C), 3.62 (s, 2H, ArCH₂C), 3.46 (t, 2H, J=6.5 Hz, OCH₂CH₂), 3.41 (t, 2H, J=6.5 Hz, OCH₂CH₂) 1.62-1.55 (m, 4H, Alkyl), 1.37-1.22 (m, 26H, Alkyl and OCH₂CH₃), 0.88 (t, 6H, J=6.8 Hz, Alkyl), 0.26 (s, 18H, Si(CH₃)₃).

Thin layer chromatography (TLC) (Merk 5554): Rf=0.65 (hexane:Et₂O=1:1 (v/v)).

### Example 67

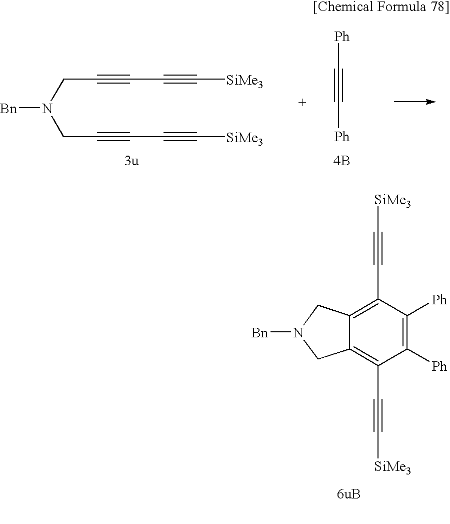

The substituted benzene 6uB was obtained from the compound 3u and compound 4B in a similar manner as in Example 4 (yield: 52%).

¹H-NMR (500 MHz, CDCl₃): δ 7.45 (d, 2H, J=7.8 Hz, Ar), 7.38 (t, 2H, J=7.8 Hz, Ar), 7.30 (t, 1H, J=7.8 Hz, Ar), 7.12-7.07 (m, 6H, Ar), 7.04-7.01 (m, 4H, Ar), 4.12 (s, 4H, ArCH₂N), 3.99 (s, 2H, ArCH₂N), −0.01 (s, 9H, Si(CH₃)₃), −0.02 (s, 9H, Si(CH₃)₃).

¹³C-NMR (125 MHz, CDCl₃): δ 139.0, 130.6, 128.8, 128.4, 127.3, 127.2, 127.0, 126.5, 126.4, 117.9, 101.9, 60.0, 59.4, −0.4.

Thin layer chromatography (TLC) (Merk 5554): Rf=0.79 (hexane:Et₂O=1:1 (v/v)).

### Example 68

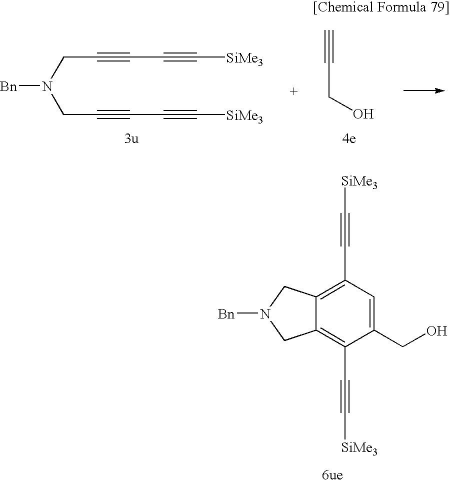

The substituted benzene 6ue was obtained from the compound 3u and compound 4e in a similar manner as in Example 4 except for stirring at room temperature for 8 hours (yield: 85%)

¹H-NMR (500 MHz, CDCl₃): δ 7.41 (d, 2H, J=7.5 Hz, Ar), 7.36 (t, 2H, J=7.5 Hz, Ar), 7.29 (t, 1H, J=7.5 Hz, Ar), 4.74 (d, 2H, J=5.5 Hz, ArCH₂OH), 4.01 (s, 4H, ArCH₂N), 3.93 (s, 2H, ArCH₂N), 2.78-2.72 (br, 1H, ArCH₂OH), 0.22 (s, 9H, Si(CH₃)₃), 0.21 (s, 9H, Si(CH₃)₃).

¹³C-NMR (125 MHz, CDCl₃): δ 134.5, 132.6, 129.7, 128.9, 128.7, 128.6, 128.4, 128.0, 127.3, 117.7, 115.7, 104.1, 102.2, 60.0, 58.8, 58.6, −0.1.

Thin layer chromatography (TLC) (Merk 5554): Rf=0.60 (hexane:Et₂O=1:1 (v/v)).

### Example 69

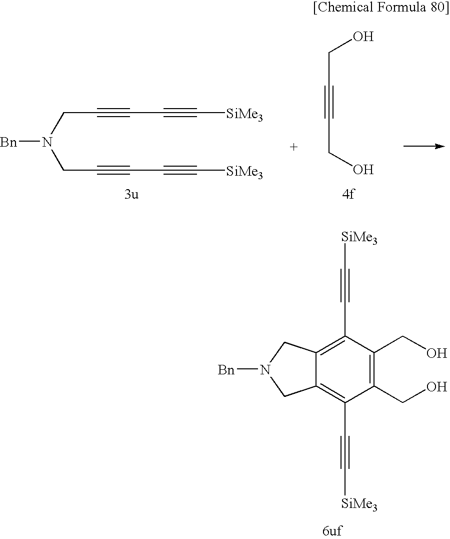

The substituted benzene 6uf was obtained from the compound 3u and compound 4f in a similar manner as in Example 4 except for stirring at room temperature for 8 hours (yield: 94%).

¹H-NMR (600 MHz, CDCl₃): δ 7.40 (d, 2H, J=7.8 Hz, Ar), 7.36 (t, 2H, J=7.8 Hz, Ar), 7.29 (t, 1H, J=7.8 Hz, Ar), 4.96 (s, 4H, ArCH₂OH), 4.03 (s, 4H, ArCH₂N), 3.93 (s, 2H, ArCH₂N), 2.78-2.72 (br, 2H, ArCH₂OH), 0.23 (s, 18H, Si(CH₃)₃).

¹³C-NMR (125 MHz, CDCl₃): δ 165.8, 149.4, 140.2, 128.7, 128.4, 125.7, 118.4, 101.6, 100.1, 61.1, 60.0, 59.4, −0.1.

Thin layer chromatography (TLC) (Merk 5554): Rf=0.26 (hexane:Et₂O=1:1 (v/v)).

### Example 70

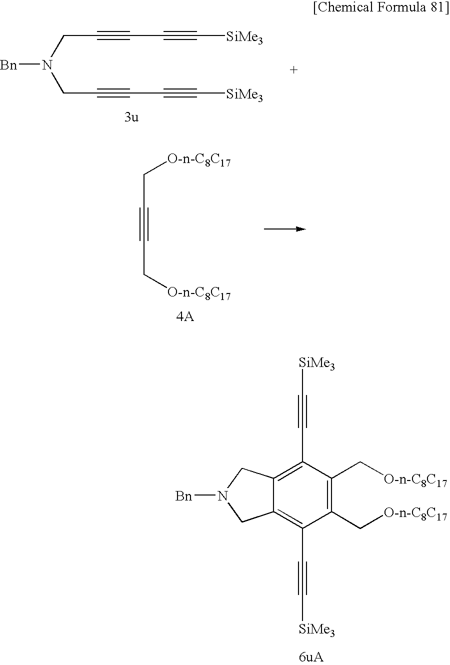

The substituted benzene 6uA was obtained from the compound 3u and compound 4A in a similar manner as in Example 4 except for stirring at room temperature for 8 hours (yield: 95%).

¹H-NMR (500 MHz, CDCl₃): δ 7.40 (d, 2H, J=7.5 Hz, Ar), 7.35 (t, 2H, J=7.5 Hz, Ar), 7.28 (t, 1H, J=7.5 Hz, Ar), 4.76 (s, 4H, ArCH₂O), 4.01 (s, 4H, ArCH₂N), 3.91 (s, 2H, ArCH₂N), 3.45 (t, 4H, J=6.2 Hz, OCH₂CH₂), 1.62-1.52 (m, 4H, Alkyl), 1.37-1.21 (m, 20H, Alkyl), 0.90-0.83 (m, 6H, Alkyl), 0.21 (s, 18H, Si(CH₃)₃).

Thin layer chromatography (TLC) (Merk 5554): Rf=0. (hexane:Et₂O=1:1 (v/v)).

### Example 71

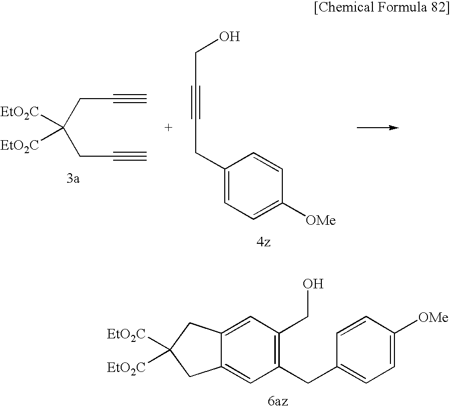

The substituted benzene 6az was obtained from the compound 3a and compound 4z in a similar manner as in Example 4 except for stirring at room temperature for 6 hours (yield: 88%).

¹H-NMR (500 MHz, CDCl₃): δ 7.23 (s, 1H, Ar), 7.04 (d, 2H, J=8.2 Hz, Ar), 6.98 (s, 1H, Ar), 6.81 (d, 2H, J=8.2 Hz, Ar), 4.59 (d, 2H, J=6.0 Hz, ArCH₂OH), 4.20 (q, 4H, J=7.2 Hz, OCH₂CH₃), 3.97 (s, 2H, ArCH₂Ar), 3.77 (s, 3H, OCH₃), 3.57 (s, 2H, ArCH₂C), 3.54 (s, 2H, ArCH₂C), 1.38-1.33 (br, 1H, ArCH₂OH), 1.25 (t, 6H, J=7.2 Hz, OCH₂CH₂).

¹³C-NMR (125 MHz, CDCl₃): δ 171.6, 157.9, 139.9, 138.4, 137.9, 137.5, 132.6, 129.5, 126.1, 124.3, 113.9, 63.3, 61.6, 60.4, 55.2, 40.3, 40.2, 37.5, 14.0.

Thin layer chromatography (TLC) (Merk 5554): Rf=0.56 (hexane:EtOAc=2:1 (v/v)).

### Example 72

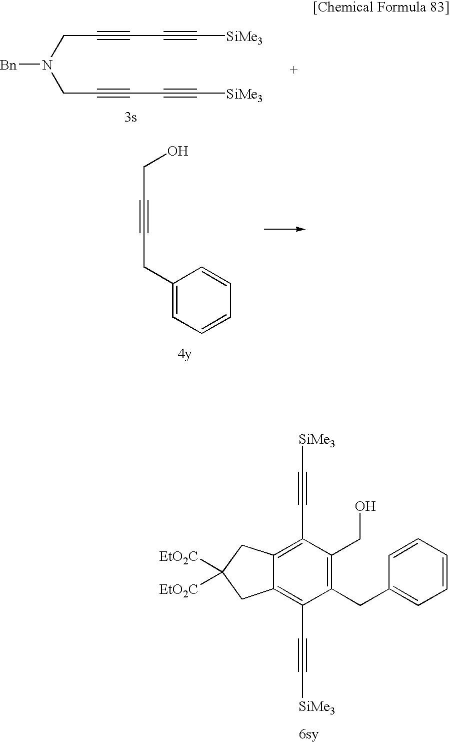

The substituted benzene 6sy was obtained from the compound 3s and compound 4y in a similar manner as in Example 4 except for stirring at room temperature for 6 hours (yield: 54%).

¹H-NMR (500 MHz, CDCl₃): δ 7.25-7.09 (m, 5H, Ar), 4.76 (d, 4H, J=6.5 Hz, ArCH₂OH), 4.35 (s, 2H, ArCH₂Ar), 4.24 (q, 4H, J=7.2 Hz, OCH₂CH₃), 3.69 (s, 2H, ArCH₂C), 3.67 (s, 2H, ArCH₂C), 1.28 (t, 6H, J=7.2 Hz, OCH₂CH₃), 0.25 (s, 9H, Si(CH₃)₃), 0.17 (s, 9H, Si(CH₃)₃).

IR (neat): 3410, 2152, 1715 cm⁻¹.

Thin layer chromatography (TLC) (Merk 5554): Rf=0.26 (hexane:EtOAc=1:5 (v/v)).

### Example 73

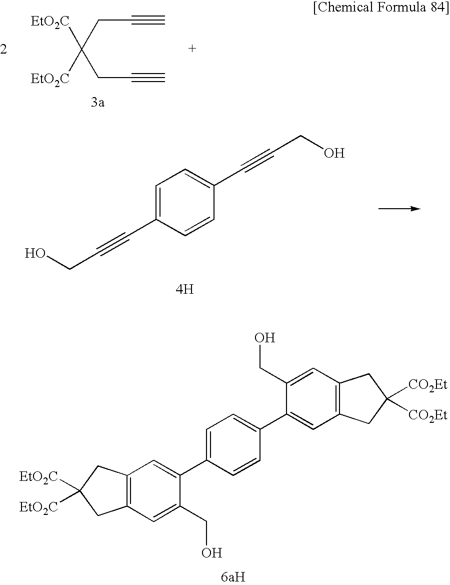

The substituted benzene 6aH was obtained from the compound 3a and compound 4H in a similar manner as in Example 4 except for stirring at room temperature for 12 hours (yield: 89%).

¹H-NMR (600 MHz, CDCl₃): δ 7.40 (s, 2H, Ar), 7.37 (s, 4H, Ar), 7.15 (s, 2H, Ar), 4,60 (d, 4H, J=5.4 Hz, ArCH₂OH), 4.22 (q, 4H, J=7.2 Hz, OCH₂CH₃), 3.65 (s, 2H, ArCH₂C), 3.62 (s, 2H, ArCH₂C), 1.66-1.60 (br, 2H, ArCH₂OH), 1.26 (t, 6H, J=7.2 Hz, OCH₂CH₃).

¹³C-NMR (150 MHz, CDCl₃): δ 171.6, 139.9, 139.74, 139.71, 139.6, 129.0, 125.8, 124.3, 63.1, 61.7, 60.5, 40.3, 40.2, 14.0.

Thin layer chromatography (TLC) (Merk 5554): Rf=0.18 (hexane:EtOAc=1:1 (v/v)).

### Example 74

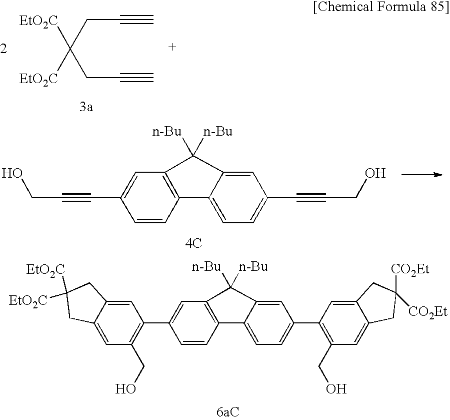

The substituted benzene 6aC was obtained from the compound 3a and compound 4C in a similar manner as in Example 4 except for stirring at room temperature for 12 hours (yield: 58%).

¹H-NMR (500 MHz, CDCl₃): δ 7.74 (d, 2H, J=7.2 Hz, Ar), 7.41 (s, 2H, Ar), 7.32 (s, 2H, Ar), 7.31 (d, 2H, J=7.2 Hz, Ar), 7.20 (s, 2H, Ar), 4.60 (d, 4H, J=5.5 Hz, ArCH₂OH), 4.23 (q, 8H, J=7.2 Hz, OCH₂CH₃), 3.66 (s, 4H, ArCH₂C), 3.64 (s, 4H, ArCH₂C), 2.00-1.95 (m, 4H, Alkyl), 1.28 (t, 12H, J=7.2 Hz, OCH₂CH₃), 1.28-1.22 (m, 4H, Alkyl), 1.13-1.05 (m, 4H, Alkyl), 0.70 (t, 6H, J=7.2 Hz, Alkyl).

¹³C-NMR (150 MHz, CDCl₃): δ 171.6, 150.9, 140.7, 139.7, 139.6, 139.5, 137.1, 127.8, 125.2, 124.2, 123.8, 119.4, 63.3, 61.7, 60.5, 55.0, 40.31, 40.30, 40.0, 26.1, 22.9, 14.0, 13.8.

Thin layer chromatography (TLC) (Merk 5554): Rf=0.41 (hexane:EtOAc=1:1 (v/v)).

### Example 75

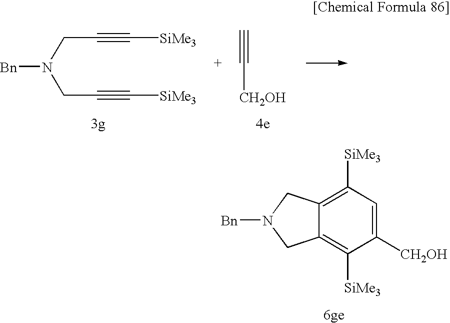

The substituted benzene 6ge was obtained from the compound 3g and compound 4e in a similar manner as in Example 4 except for stirring at room temperature for 12 hours (yield: 88%).

¹H-NMR (500 MHz, CDCl₃): δ 7.39 (d, 2H, J=7.2 Hz, Ph), 7.36 (s, 1H, Ar), 7.35 (t, 2H, J=7.2 Hz, Ph), 7.28 (t, 1H, J=7.2 Hz, Ph), 4.71 (s, 2H, ArCH₂OH), 3.95 (s, 2H, PhCH₂N), 3.90 (s, 4H, ArCH₂N), 1.73-1.47 (br, 1H, CH₂OH), 0.33 (s, 9H, Si(CH₃)₃), 0.24 (s, 9H, Si(CH₃)₃).

Thin layer chromatography (TLC) (Merk 5554): Rf=0.24 (hexane:Et₂O=1:1 (v/v)).

### Example 76

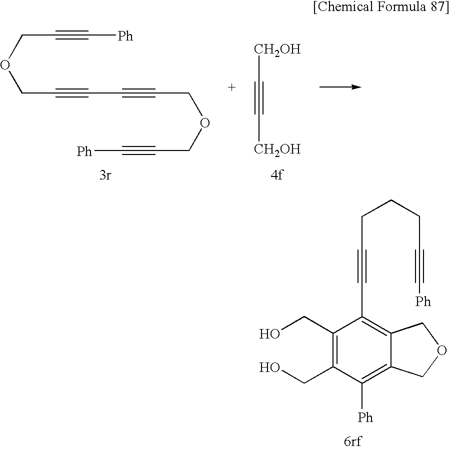

The substituted benzene 6rf was obtained from the compound 3r and compound 4f (used as much as 5 equivalents) in a similar manner as in Example 4 except for stirring at room temperature for 12 hours (yield: 82%).

¹H-NMR (500 MHz, CDCl₃): δ 7.30-7.47 (m, 10H, Ph), 5.20 (s, 2H), 5.06 (s, 2H), 4.90 (s, 2H), 4.58 (s, 2H), 4.54 (s, 4H), 3.32 (bs, 2H).

¹³C-NMR (68 MHz, CDCl₃): δ 141.8, 141.2, 138.4, 137.7, 137.3, 131.7, 128.5, 128.2, 127.8, 122.2, 115.7, 92.19, 87.13, 84.00, 82.06, 74.54, 74.44, 61.35, 59.73, 57.55, 57.37.

Thin layer chromatography (TLC) (Merk 5554): Rf=0.20 (hexane:AcOEt=10:1 (v/v)).

### Example 77

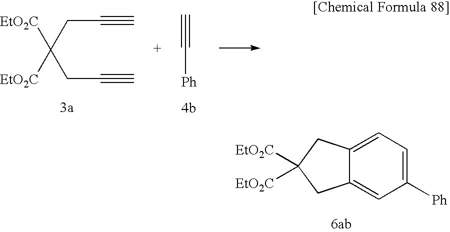

Zinc powder (6.5 mg, 0.10 mmol), the compound 3a (1.0 mmol) and the compound 4b (1.3 mmol) were dissolved in CH₃CN (2.5 mL), followed by the addition of a suspension of CoCl₂-6H₂O (11.9 mg, 0.05 mmol) and dipimp (16.0 mg, 0.06 mmol) in CH₃CN (1.5 mL). The resulting mixed solution was stirred at room temperature for 12 hours. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzene 6ab (yield: 80%).

### Example 78

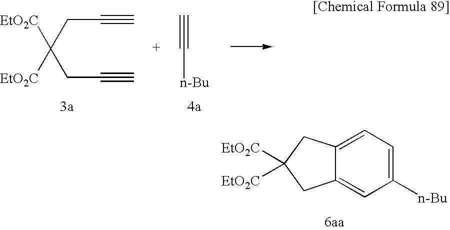

The substituted benzene 6aa was obtained from the compound 3a and compound 4a in a similar manner as in Example 77 (yield: 52%).

### Example 79

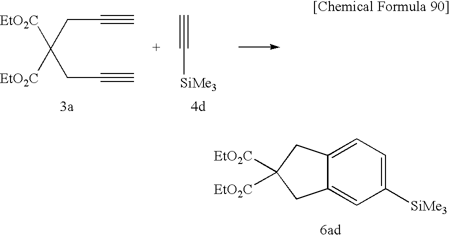

The substituted benzene 6ad was obtained from the compound 3a and compound 4d in a similar manner as in Example 77 (yield: 48%).

### Example 80

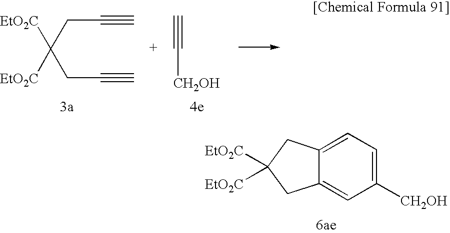

Zinc powder (6.5 mg, 0.10 mmol), the compound 3a (1.0 mmol) and the compound 4e (3.0 mmol) were dissolved in THF (2.5 mL), followed by the addition of a solution of RuCl₃-3H₂O (13.1 mg, 0.05 mmol) and dipimp (16.0 mg, 0.06 mmol) in THF (1.5 mL). The resulting mixed solution was stirred at room temperature for 12 hours. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzene 6ae (yield: 70%).

### Example 81

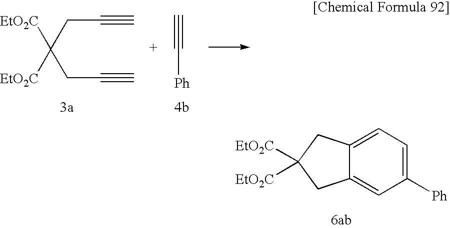

The substituted benzene 6ab was obtained from the compound 3a and compound 4b (used as much as 1.3 equivalents) in a similar manner as in Example 80 (yield: 67%).

### Example 82

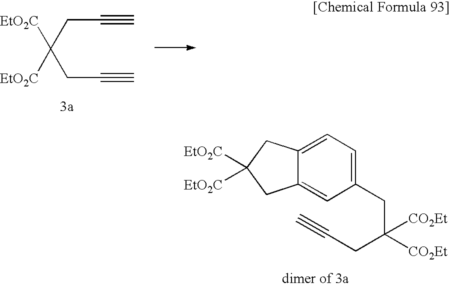

The dimer of the compound 3a was obtained as a substituted benzene from the compound 3a in a similar manner as in Example 80 (yield: 88%).

¹H-NMR (600 MHz, CDCl₃): δ 7.08 (d, 1H, J=7.8 Hz, Ar), 7.00 (s, 1H, Ar), 6.96 (d, 1H, J=7.8 Hz, Ar), 4.16-4.26 (m, 8H, OCH₂CH₃), 3.54 (s, 4H, cyclic ArCH₂C), 3.35 (s, 2H, acyclic ArCH₂C), 2.66 (s, 2H, C≡CCH₂), 2.14 (t, 1H, J=2.4 Hz, C≡CH), 1.22-1.28 (m, 12H, OCH₂CH₃).

¹³C-NMR (150 MHz, CDCl₃): δ 171.5, 169.6, 140.2, 138.8, 134.2, 128.5, 125.5, 124.0, 79.3, 72.0, 61.6 (4C), 60.3, 58.0, 40.3, 37.0, 22.0, 13.94, 13.91.

IR (neat): 3275, 2982, 2940, 1738, 1715, 1242, 1184, 1161 cm⁻¹.

Anal. Calcd for C₂₆H₃₂O₈: C, 66.09; H, 6.83. Found: C, 66.25; H, 7.03.

### Example 83

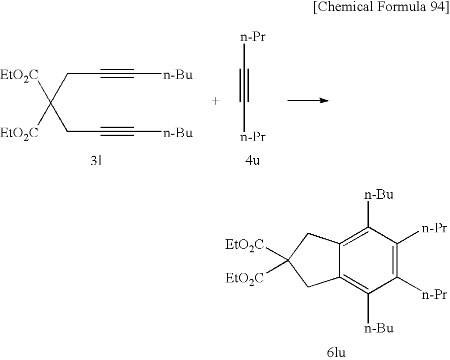

Zinc powder (6.5 mg, 0.10 mmol), the compound 3l (1.0 mmol), the compound 4u (3.0 mmol) and silver trifluoromethanesulfonate (25.7 mg, 0.10 mmol) were dissolved in THF (2.5 mL), followed by the addition of a solution of CoCl₂-6H₂O (11.9 mg, 0.05 mmol) and dipimp (16.0 mg, 0.06 mmol) in THF (1.5 mL). The resulting mixed solution was stirred at room temperature for 3 hours. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzene 61u (yield: 72%).

¹H-NMR (500 MHz, CDCl₃): δ 4.20 (q, 4H, J=8.2 Hz, OCH₂CH₃), 3.52 (s, 4H, ArCH₂C), 2.47-2.45 (m, 8H, ArCH₂), 1.47-1.41 (m, 12H, methelyne-Hs), 1.27 (t, 6H, J=7.2 Hz, OCH₂CH₃), 1.03 (t, 6H, J=7.5 Hz, CH₂CH₂CH₂CH₃), 0.96 (t, 6H, J=7.0 Hz, CH₂CH₂CH₃).

¹³C-NMR (68 MHz, CDCl₃): δ 171.7, 137.4, 136.0, 134.0, 61.48, 59.70, 39.59, 32.59, 31.61, 30.36, 25.12, 23.36, 15.03, 14.03, 13.93.

Thin layer chromatography (TLC) (Merk 5554): Rf=0.85 (hexane:AcOEt=10:1 (v/v)).

### Example 84

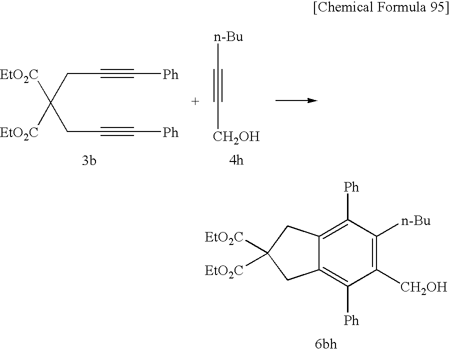

The substituted benzene 6bh was obtained from the compound 3b and compound 4h in a similar manner as in Example 83 (yield: 92%).

¹H-NMR (500 MHz, CDCl₃): δ 7.30-7.50 (m, 10H, Ph), 4.48 (d, 2H, J=8.2 Hz, ArCH₂OH), 4.10 (q, 4H, J=8.4 Hz, OCH₂CH₃), 3.27 (s, 2H, ArCH₂C), 3.23 (s, 2H, ArCH₂C), 2.56 (t, 2H, J=Hz, ArCH₂CH₂), 1.25-1.35 (m, 4H, methelyne-Hs), 1.15 (t, 6H, J=7.5 Hz, OCH₂CH₃), 0.86 (t, 1H, J=7.4 Hz, OH), 0.67 (t, 3H, J=7.4 Hz, CH₃).

¹³C-NMR (150 MHz, CDCl₃): δ 171.5, 140.1, 139.6, 138.3, 136.4, 135.1, 131.5, 129.5, 129.0, 128.5, 128.3, 127.5, 127.2, 126.9, 61.56, 59.69, 58.17, 40.79, 40.55, 34.20, 29.53, 22.98, 13.96, 13.52.

Thin layer chromatography (TLC) (Merk 5554): Rf=0.20 (hexane:AcOEt=10:1 (v/v)).

### Example 85

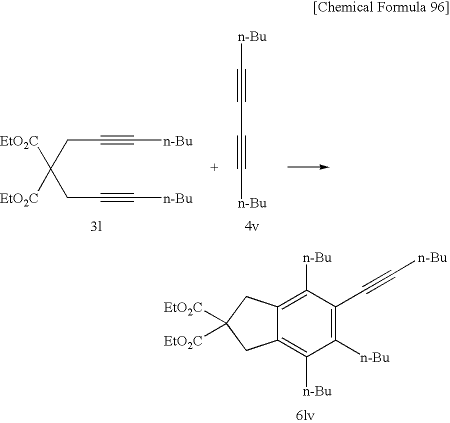

The substituted benzene 61v was obtained from the compound 3l and compound 4v in a similar manner as in Example 83 except for stirring at room temperature for 2 hours (yield: 60%).

¹H-NMR (500 MHz, CDCl₃): δ 4.20 (q, 4H, J=8.2 Hz, OCH₂CH₃), 3.52 (s, 2H, ArCH₂C), 3.50 (s, 2H, ArCH₂C), 2.74 (t, 2H, J=8.0 Hz, ArCH₂), 2.68 (t, 2H, J=8.0 Hz, ArCH₂), 2.50 (t, 2H, J=8.0 Hz, ArCH₂), 2.46 (t, 2H, J=7.0 Hz, C≡CCH₂), 1.42-1.58 (m, 16H, methelyne-Hs), 1.25 (t, 6H, J=7.8 Hz, OCH₂CH₃), 0.97-0.92 (m, 12H, CH₂CH₂CH₂CH₃).

¹³C-NMR (68 MHz, CDCl₃): δ 171.5, 141.7, 138.0, 137.9, 135.3, 133.4, 122.1, 95.90, 78.41, 61.55, 59.82, 39.68, 39.26, 33.03, 32.52, 32.07, 31.78, 31.08, 30.08, 23.34, 23.18, 23.07, 21.97, 19.32, 14.02, 13.93, 13.60.

Thin layer chromatography (TLC) (Merk 5554): Rf=0.80 (hexane:AcOEt=10:1 (v/v)).

### Example 86

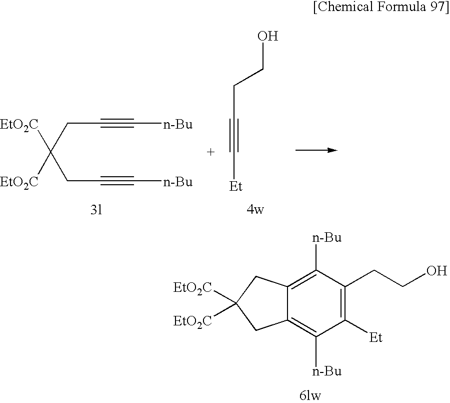

The substituted benzene 61w was obtained from the compound 3l and compound 4w in a similar manner as in Example 83 except for stirring at room temperature for 2 hours (yield: 82%).

¹H-NMR (500 MHz, CDCl₃): δ 4.20 (q, 4H, J=8.0 Hz, OCH₂CH₃), 3.73 (t, 2H, ArCH₂CH₂OH), 3.54 (bs, 4H, ArCH₂C), 2.91 (t, 2H, J=8.0 Hz, ArCH₂CH₂OH), 2.63 (q, 2H, J=7.5 Hz, ArCH₂CH₃), 2.52-2.57 (m, 4H, ArCH₂), 1.42-1.47 (m, 8H, methelyne-Hs), 1.26 (t, 3H, J=7.5 Hz, OCH₂CH₃), 1.13 (t, 3H, J=7.8 Hz, CH₂CH₃), 0.95 (t, 6H, J=7.6 Hz, CH₂CH₂CH₂CH₃).

¹³C-NMR (68 MHz, CDCl₃): δ 171.6, 139.5, 137.1, 136.2, 135.0, 134.2, 131.6, 63.52, 61.55, 59.65, 39.59, 39.56, 32.66, 32.61, 32.14, 30.49, 30.26, 23.32, 22.05, 15.91, 14.02, 13.98, 13.96.

Thin layer chromatography (TLC) (Merk 5554): Rf=0.25 (hexane:AcOEt=10:1 (v/v)).

### Example 87

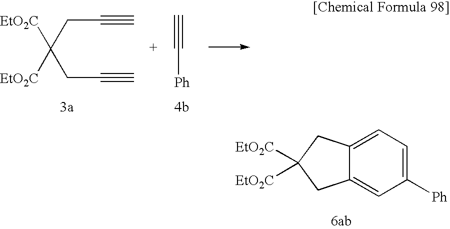

The substituted benzene 6ab was obtained from the compound 3a and compound 4b in a similar manner as in Example 83 except for stirring at room temperature for 30 minutes (yield: 92%).

### Example 88

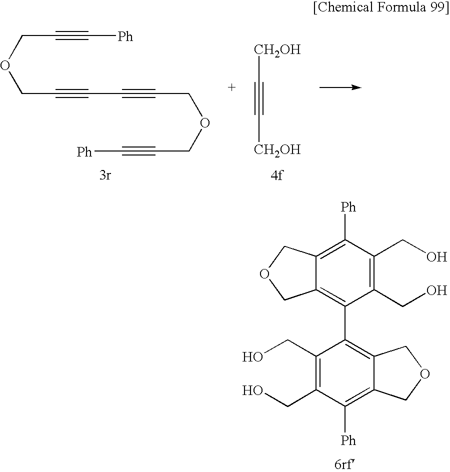

The substituted benzene 6rf′ was obtained from the compound 3r and compound 4f (used as much as 6 equivalents) in a similar manner as in Example 83 except for stirring at room temperature for 10 hours (yield: 92%).

¹H-NMR (500 MHz, CDCl₃): δ 7.23-7.45 (m, 5H, Ph), 5.40 (bs, 2H), 5.04 (d, 2H), 4.85 (t, 2H), 4.66-4.74 (m, 4H), 4.54 (d, 1H), 4.22 (d, 1H).

¹³C-NMR (68 MHz, CDCl₃): δ 138.5, 138.0, 137.8, 137.3, 136.8, 131.2, 128.5, 128.3, 127.6, 74.31, 74.06, 59.85, 58.99.

Thin layer chromatography (TLC) (Merk 5554): Rf=0.10 (hexane:AcOEt=10:1 (v/v)).

### Example 89

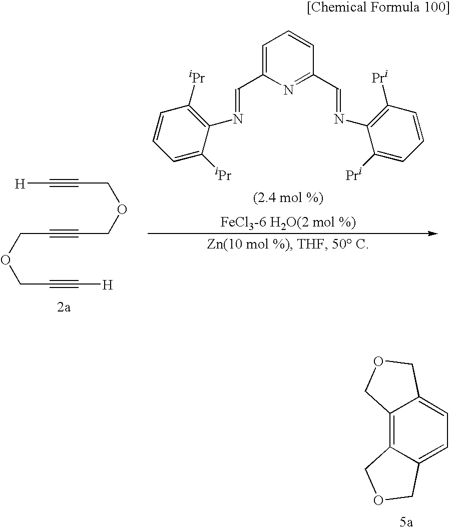

Zinc powder (6.5 mg, 0.10 mmol) and the compound 2a (1.0 mmol) were dissolved in THF (2.5 mL), followed by the addition of a solution of FeCl₃-6H₂O (5.4 mg, 0.02 mmol) and 2,6-bis(2,6-diisopropylphenyliminomethyl)pyridine (10.9 mg, 0.024 mmol) in THF (1.5 mL). The resulting mixed solution was stirred at 50° C. for 24 hours. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzene 5a (yield: 62%).

It is to be noted that 2,6-bis(2,6-diisopropylphenyliminomethyl)pyridine was synthesized in accordance with the procedure described in J. Am. Chem. Soc., 121, (1999), 8728.

### Example 90

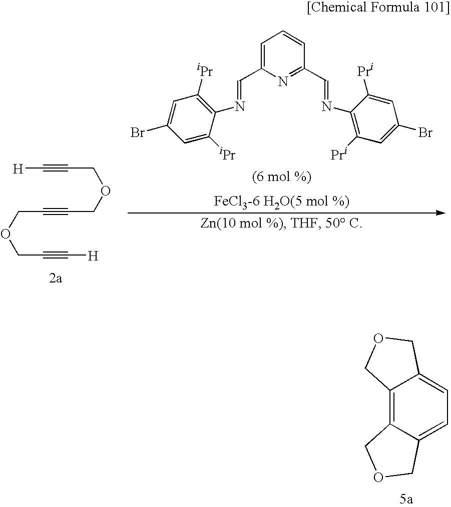

Zinc powder (6.5 mg, 0.10 mmol) and the compound 2a (1.0 mmol) were dissolved in THF (2.5 mL), followed by the addition of a solution of FeCl₃-6H₂O (13.5 mg, 0.05 mmol) and 2,6-bis(4-bromo-2,6-diisopropylphenyliminomethyl)pyridine (36.7 mg, 0.06 mmol) in THF (1.5 mL). The resulting mixed solution was stirred at 50° C. for 48 hours. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzene 5a (yield: 52%).

It is to be noted that 2,6-bis(4-bromo-2,6-diisopropylphenyliminomethyl)pyridine was synthesized in accordance with the procedure described in J. Am. Chem. Soc., 121; (1999), 8728.

### Example 91

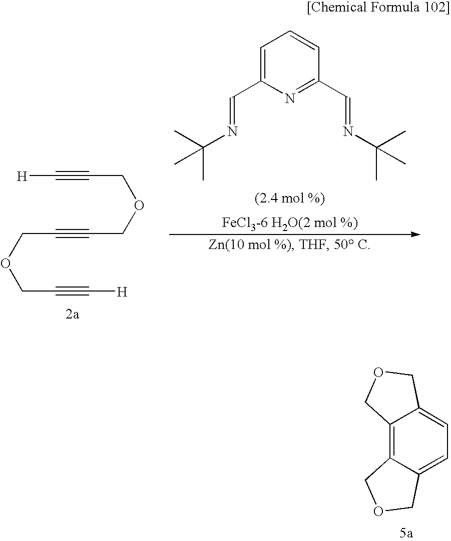

Zinc powder (6.5 mg, 0.10 mmol) and the compound 2a (1.0 mmol) were dissolved in THF (2.5 mL), followed by the addition of a solution of FeCl₃-6H₂O (5.4 mg, 0.02 mmol) and 2,6-bis(t-butyliminomethyl)pyridine (5.9 mg, 0.024 mmol) in THF (1.5 mL). The resulting mixed solution was stirred at 50° C. for 24 hours. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzene 5a (yield: 18%).

It is to be noted that 2,6-bis(t-butyliminomethyl)-pyridine was synthesized in accordance with the procedure described in J. Am. Chem. Soc., 121, (1999), 8728.

### Example 92

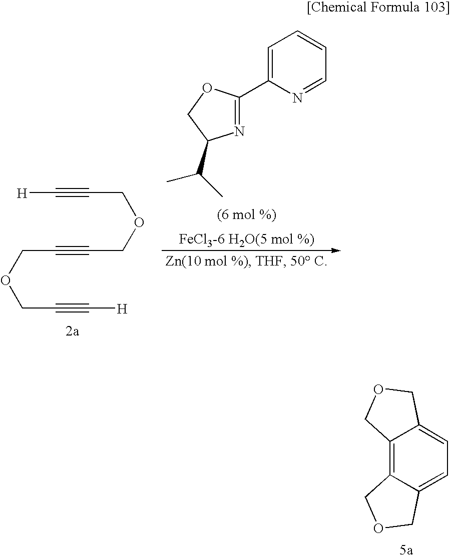

Zinc powder (6.5 mg, 0.10 mmol) and the compound 2a (1.0 mmol) were dissolved in THF (2.5 mL), followed by the addition of a solution of FeCl₃-6H₂O (13.5 mg, 0.05 mmol) and 2-(4-isopropyl-4,5-dihydrooxazol-2-yl)pyridine (11.4 mg, 0.06 mmol) in THF (1.5 mL). The resulting mixed solution was stirred at 50° C. for 24 hours. After completion of the reaction, diethyl ether (10 mL) was added, and the resulting mixture was filtered through “Celite”. The filtrate was concentrated under reduced pressure, and the concentrate was purified by chromatography on a silica gel column to obtain the substituted benzene 5a (yield: 26%).

It is to be noted that 2-(4-isopropyl-4,5-dihydro-oxazol-2-yl)pyridine was synthesized in accordance with the procedure described in Chemische Berichte, (1991), 124(5), 1173-80.

1. A process for producing a substituted benzene by subjecting a triple bond in an alkyne to intramolecular and/or intermolecular trimerization in the presence of a transition metal catalyst to obtain a substituted benzene compound, wherein
said transition metal catalyst is prepared in a reaction system from an iminomethylpyridine represented by the following formula (1) or formula (2), a transition metal salt or a hydrate thereof and a reducing agent to conduct said trimerization: 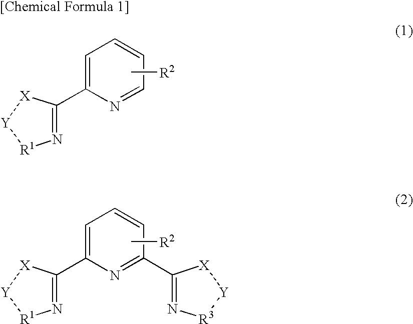
wherein R¹ and R³ each independently represent a linear or cyclic C₁-C₂₀ aliphatic hydrocarbon group or a C₆-C₂₀ aromatic hydrocarbon group, R² represents a hydrogen atom, a linear or cyclic C₁-C₂₀ aliphatic hydrocarbon group or a C₆-C₂₀ aromatic hydrocarbon group, X represents a hydrogen atom, O, S, NR⁴, CH₂, CHR⁴ or CR⁴₂ in which each R⁴ independently represents a linear or cyclic C₁-C₂₀ aliphatic hydrocarbon group or a C₆-C₂₀ aromatic hydrocarbon group, and Y represents O, S, NR⁴, CH₂, CHR⁴ or CR⁴₂ in which each R⁴ independently represents a linear or cyclic C₁-C₂₀ aliphatic hydrocarbon group or a C₆-C₂₀ aromatic hydrocarbon group, with a proviso that, when X is a hydrogen atom, Y is absent and that X and Y do not represent O and/or NR⁴ at a same time.

2. The process according to claim 1, wherein said hydrate of said transition metal salt is represented by the following formula (3):
MZₘ—(H₂O)ₙ (3)
wherein, M represents Ti, Zr, V, Nb, Ta, Cr, Mo, W, Mn, Fe, Ru, Co, Rh, Ir, Ni, Pd or Pt,
Z represents Cl, Br, I, NO₂, CN, OAc, OBz, OTf, NTf₂, ClO₄, BF₄, PF₆ or acac, in which Ac means an acetyl group, Bz means a benzoyl group, Tf means a trifluoromethanesulfonyl group, and acac means an acetylacetonato group, and m is a number corresponding to a valency of M forming said salt, and n is a number corresponding to a hydrate existing depending on a combination of M and Z.

3. The process according to claim 2, wherein M is Fe, Co, Ni, Pd, Ru or Rh.

4. The process according to claim 2 or 3, wherein Z is Cl, Br or I.

5. The process according to claim 1, wherein said transition metal salt or said hydrate thereof is FeCl₂, FeCl₃, CoCl₂, CoCl₃, NiCl₂, FeCl₃.6H₂O, CoCl₂.6H₂O, or NiCl₂.6H₂O.

6. The process according to any one of claims 1 to 5, wherein said reducing agent is Zn.

7. The process according to any one of claims 1 to 6, wherein said alkyne is a compound represented by the following formula (4), and triple bonds in said compound are subjected to intramolecular trimerization: 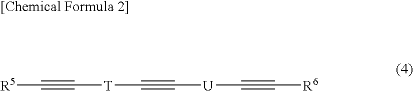
wherein, R⁵ and R⁶ each independently represent a hydrogen atom, alkoxy group, hydroxyalkyl group, alkylcarbonyloxy group, amino group, alkoxycarbonyl group, amide group, phosphate ester group, phosphine oxide group, borate ester group, trialkylsilyl group, trialkylstannyl group, linear or cyclic C₁-C₂₀ aliphatic hydrocarbon group, or C₆-C₂₀ aromatic hydrocarbon group in which said aliphatic or aromatic hydrocarbon group may contain at least one of hydroxyl groups, amino groups, alkylcarbonyloxy groups, ether groups, amide groups, cyano groups, nitro groups, phosphate ester groups, phosphine oxide groups, borate ester groups, trialkylsilyl groups, trialkylstannyl groups, dialkylsulfide groups, thiol groups, sulfoxide groups, sulfone groups and sulfonate ester groups, and
T and U each independently represent —(CR⁷)ₖ₁—W—, —W—(CR⁷₂)ₖ₁— or —(CR⁷₂)ₖ₂—W—(CR⁷₂)ₖ₃—, in which W represents O, S, NR⁷, SiR⁷₂, BR⁷ or CR⁷₂, each R⁷ independently represents a hydrogen atom, linear or cyclic C₁-C₂₀ aliphatic hydrocarbon group, C₆-C₂₀ aromatic hydrocarbon group or alkoxycarbonyl group, k₁ stands for 2 or 3, and k₂ and k₃ are 1 or 2 and satisfy k₂+k₃=2 or 3.

8. The process according to any one of claims 1 to 6, wherein said alkyne is a combination of a compound represented by the following formula (5) and a compound represented by the following formula (6), and triple bonds in said compounds are subjected to intramolecular and intermolecular trimerization: 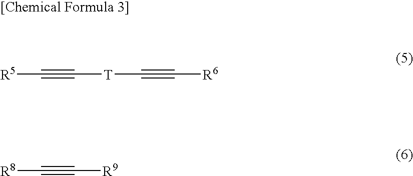
wherein, R⁵, R⁶, R⁸ and R⁹ each independently represent a hydrogen atom, alkoxy group, hydroxyalkyl group, alkylcarbonyloxy group, amino group, alkoxycarbonyl group, amide group, phosphate ester group, phosphine oxide group, borate ester group, trialkylsilyl group, trialkylstannyl group, linear or cyclic C₁-C₂₀ aliphatic hydrocarbon group, or C₆-C₂₀ aromatic hydrocarbon group in which said aliphatic or aromatic hydrocarbon group may contain at least one of hydroxyl groups, amino groups, alkylcarbonyloxy groups, ether groups, amide groups, cyano groups, nitro groups, phosphate ester groups, phosphine oxide groups, borate ester groups, trialkylsilyl groups, trialkylstannyl groups, dialkylsulfide groups, thiol groups, sulfoxide groups, sulfone groups and sulfonate ester groups, and
T represents —(CR⁷₂)ₖ₁—W—, —W—(CR⁷₂)ₖ₁— or —(CR⁷)ₖ₂—W—(CR⁷₂)ₖ₃—, in which W represents O, S, NR⁷, SiR⁷₂, BR⁷ or CR⁷₂, each R⁷ independently represents a hydrogen atom, linear or cyclic C₁-C₂₀ aliphatic hydrocarbon group, C₆-C₂₀ aromatic hydrocarbon group or alkoxycarbonyl group, k₁ stands for 2 or 3, and k₂ and k₃ are 1 or 2 and satisfy k₂+K₃=2 or 3.

9. The process according to any one of claims 1 to 6, wherein said alkyne is a compound represented by the following formula (7), and a triple bond in said compound is subjected to intermolecular trimerization: 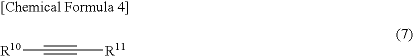
wherein, R¹⁰ and R¹¹ each independently represent a hydrogen atom, alkoxy group, hydroxyalkyl group, amino group, alkylcarbonyloxy group, alkoxycarbonyl group, amide group, phosphate ester group, phosphine oxide group, borate ester group, trialkylsilyl group, trialkylstannyl group, linear or cyclic C₁-C₂₀ aliphatic hydrocarbon group, or C₆-C₂₀ aromatic hydrocarbon group in which said aliphatic or aromatic hydrocarbon group may contain at least one of hydroxyl groups, amino groups, alkylcarbonyloxy groups, ether groups, amide groups, cyano groups, nitro groups, phosphate ester groups, phosphine oxide groups, borate ester groups, trialkylsilyl groups, trialkylstannyl groups, dialkylsulfide groups, thiol groups, sulfoxide groups, sulfone groups and sulfonate ester groups, with a proviso that R¹⁰ and R¹¹ do not represent hydrogen atoms at a same time in all three molecules.

10. The process according to any one of claims 1 to 9, wherein a silver sulfonate compound selected from the group consisting of AgOSO₂R in which R represents a methyl group, phenyl group, 4-methylphenyl group, trifluoromethyl group or 4-trifluoromethylphenyl group, AgBF₄ and AgPF₆ is added further.

11. The process according to claim 10, wherein said silver sulfonate compound is added in an amount of from 0.2 to 5 equivalents per equivalent of said transition metal salt or said hydrate thereof.
